SEQUENCE LISTING (104 sequences)

SEQ ID NO: 1 - protein, 21 aa, Homo sapiens
HPLKPDNQPFPQSVSESCPGK

SEQ ID NO: 2 - protein, 21 aa, Homo sapiens
HPLKPDIQPFPQSVSESCPGK

SEQ ID NO: 3 - protein, 19 aa, Homo sapiens
GDTSTIYTNCWVTGWGFSK

SEQ ID NO: 4 - protein, 14 aa, Homo sapiens
GDTSTIYTNCWVTR

SEQ ID NO: 5 - DNA, 59 bp, unidentified
  source 1..59: mol_type = other DNA
atgctagcgatcgtacgatcgatcggtgcttattattatagccggtatatcgatgccta

SEQ ID NO: 6 - DNA, 59 bp, unidentified; identical to SEQ ID NO: 5
  source 1..59: mol_type = other DNA

SEQ ID NO: 7 - protein, 6 aa, Homo sapiens
MLAIVK

SEQ ID NO: 8 - protein, 9 aa, Homo sapiens
RSIGAYYYS

SEQ ID NO: 9 - protein, 5 aa, Homo sapiens
RYIDA

SEQ ID NO: 10 - protein, 25 aa, Homo sapiens
EVYMPSSIFQDDFVIPDISEPGTWK

SEQ ID NO: 11 - protein, 17 aa, Homo sapiens
PVAFSVVPTAAAAVSLK

SEQ ID NO: 12 - protein, 35 aa, Homo sapiens
EYLIMGLDGATYDLEGHPQYLLDSNSWIEEMPSER

SEQ ID NO: 13 - protein, 18 aa, Homo sapiens
AVGSGATFSHYYYMILSR

SEQ ID NO: 14 - protein, 25 aa, Homo sapiens
ITPGKPYILTVPGHLDEMQLDIQAR

SEQ ID NO: 15 - protein, 10 aa, Homo sapiens
LGQYASPTAK

SEQ ID NO: 16 - protein, 10 aa, Homo sapiens
KYVLPNFEVK

SEQ ID NO: 17 - protein, 7 aa, Homo sapiens
EFHLHLR

SEQ ID NO: 18 - protein, 10 aa, Homo sapiens
SFFPENWLWR

SEQ ID NO: 19 - protein, 24 aa, Homo sapiens
FQILTLWLPDSLTTWEIHGLSLSK

SEQ ID NO: 20 - protein, 23 aa, Homo sapiens
YVSHFETEGPHVLLYFDSVPTSR

SEQ ID NO: 21 - protein, 26 aa, Homo sapiens
LHLETDSLALVALGALDTALYAAGSK

SEQ ID NO: 22 - protein, 12 aa, Homo sapiens
LNMGITDLQGLR

SEQ ID NO: 23 - protein, 11 aa, Homo sapiens
VDVQAGACEGK

SEQ ID NO: 24 - protein, 11 aa, Homo sapiens
GLCVATPVQLR

SEQ ID NO: 25 - protein, 8 aa, Homo sapiens
NNVPCSPK

SEQ ID NO: 26 - protein, 16 aa, Homo sapiens
FEQLELRPVLYNYLDK

SEQ ID NO: 27 - protein, 19 aa, Homo sapiens
EGAIHREELVYELNPLDHR

SEQ ID NO: 28 - protein, 17 aa, Homo sapiens
GHLFLQTDQPIYNPGQR

SEQ ID NO: 29 - protein, 9 aa, Homo sapiens
FGLLDEDGK

SEQ ID NO: 30 - protein, 15 aa, Homo sapiens
HLVPGAPFLLQALVR

SEQ ID NO: 31 - protein, 9 aa, Homo sapiens
SHALQLNNR

SEQ ID NO: 32 - protein, 8 aa, Homo sapiens
TYNVLDMK

SEQ ID NO: 33 - protein, 9 aa, Homo sapiens
YVLPNFEVK

SEQ ID NO: 34 - protein, 8 aa, Homo sapiens
GQIVFMNR

SEQ ID NO: 35 - protein, 9 aa, Homo sapiens
LELSVDGAK

SEQ ID NO: 36 - protein, 21 aa, Homo sapiens
VGLSGMAIADVTLLSGFHALR

SEQ ID NO: 37 - protein, 26 aa, Homo sapiens
LLLFSPSVVHLGVPLSVGVQLQDVPR

SEQ ID NO: 38 - protein, 20 aa, Homo sapiens
PYILTVPGHLDEMQLDIQAR

SEQ ID NO: 39 - protein, 14 aa, Homo sapiens
GPEVQLVAHSPWLK

SEQ ID NO: 40 - protein, 13 aa, Homo sapiens
GSFEFPVGDAVSK

SEQ ID NO: 41 - protein, 10 aa, Homo sapiens
FGLLDEDGKK

SEQ ID NO: 42 - protein, 9 aa, Homo sapiens
GLQDEDGYR

SEQ ID NO: 43 - protein, 14 aa, Homo sapiens
TTNIQGINLLFSSR

SEQ ID NO: 44 - protein, 21 aa, Homo sapiens
VDFTLSSERDFALLSLQVPLK

SEQ ID NO: 45 - protein, 12 aa, Homo sapiens
LVNGQSHISLSK

SEQ ID NO: 46 - protein, 13 aa, Homo sapiens
EELVYELNPLDHR

SEQ ID NO: 47 - protein, 10 aa, Homo sapiens
VGDTLNLNLR

SEQ ID NO: 48 - protein, 13 aa, Homo sapiens
EMSGSPASGIPVK

SEQ ID NO: 49 - protein, 9 aa, Homo sapiens
PVQGVAYVR

SEQ ID NO: 50 - protein, 12 aa, Homo sapiens
DFALLSLQVPLK

SEQ ID NO: 51 - protein, 8 aa, Homo sapiens
VEYGFQVK

SEQ ID NO: 52 - protein, 7 aa, Homo sapiens
VFALDQK

SEQ ID NO: 53 - protein, 9 aa, Homo sapiens
AEFQDALEK

SEQ ID NO: 54 - protein, 9 aa, Homo sapiens
VDFTLSSER

SEQ ID NO: 55 - protein, 9 aa, Homo sapiens
ITQVLHFTK

SEQ ID NO: 56 - protein, 13 aa, Homo sapiens
GLEEELQFSLGSK

SEQ ID NO: 57 - protein, 8 aa, Homo sapiens
VVEEQESR

SEQ ID NO: 58 - protein, 19 aa, Homo sapiens
ALEILQEEDLIDEDDIPVR

SEQ ID NO: 59 - protein, 22 aa, Homo sapiens
ASAGLLGAHAAAITAYALTLTK

SEQ ID NO: 60 - protein, 11 aa, Homo sapiens
ADGSYAAWLSR

SEQ ID NO: 61 - protein, 9 aa, Homo sapiens
QGSFQGGFR

SEQ ID NO: 62 - protein, 13 aa, Homo sapiens
AEMADQASAWLTR

SEQ ID NO: 63 - protein, 30 aa, Homo sapiens
NPSDPMPQAPALWIETTAYALLHLLLHEGK

SEQ ID NO: 64 - protein, 39 aa, Homo sapiens
SMQGGLVGNDETVALTAFVTIALHHGLAVFQDEGAEPLK

SEQ ID NO: 65 - protein, 12 aa, Homo sapiens
TEQWSTLPPETK

SEQ ID NO: 66 - protein, 15 aa, Homo sapiens
VLSLAQEQVGGSPEK

SEQ ID NO: 67 - protein, 23 aa, Homo sapiens
TLEIPGNSDPNMIPDGDFNSYVR

SEQ ID NO: 68 - protein, 9 aa, Homo sapiens
DHAVDLIQK

SEQ ID NO: 69 - protein, 8 aa, Homo sapiens
ANSFLGEK

SEQ ID NO: 70 - protein, 12 aa, Homo sapiens
DSSTWLTAFVLK

SEQ ID NO: 71 - protein, 27 aa, Homo sapiens
LQETSNWLLSQQQADGSFQDPCPVLDR

SEQ ID NO: 72 - protein, 25 aa, Homo sapiens
STQDTVIALDALSAYWIASHTTEER

SEQ ID NO: 73 - protein, 26 aa, Homo sapiens
VTASDPLDTLGSEGALSPGGVASLLR

SEQ ID NO: 74 - protein, 8 aa, Homo sapiens
EFMSQGNK

SEQ ID NO: 75 - protein, 26 aa, Homo sapiens
QRPPDLDTSSNAVDLLFFTDESGDSR

SEQ ID NO: 76 - protein, 11 aa, Homo sapiens
YTTTMGVNTYK

SEQ ID NO: 77 - protein, 18 aa, Homo sapiens
TLDEFTIIQNLQPQYQFR

SEQ ID NO: 78 - protein, 12 aa, Homo sapiens
LFGEVTSPLFPK

SEQ ID NO: 79 - protein, 7 aa, Homo sapiens
YTTEIIK

SEQ ID NO: 80 - protein, 19 aa, Homo sapiens
PYPNNFETTTVITVPTGYR

SEQ ID NO: 81 - protein, 8 aa, Homo sapiens
NLPNGDFR

SEQ ID NO: 82 - protein, 9 aa, Homo sapiens
VLNYVDWIK

SEQ ID NO: 83 - protein, 8 aa, Homo sapiens
PVNPVEQR

SEQ ID NO: 84 - protein, 7 aa, Homo sapiens
GYGFYTK

SEQ ID NO: 85 - protein, 8 aa, Homo sapiens
DYFIATCK

SEQ ID NO: 86 - protein, 7 aa, Homo sapiens
GLTLHLK

SEQ ID NO: 87 - protein, 14 aa, Homo sapiens
LPVANPQACENWLR

SEQ ID NO: 88 - protein, 16 aa, Homo sapiens
ESEQGVYTCTAQGIWK

SEQ ID NO: 89 - protein, 20 aa, Homo sapiens
LVFQQFDLEPSEGCFYDYVK

SEQ ID NO: 90 - protein, 27 aa, Homo sapiens
FLEPFDIDDHQQVHCPYDQLQIYANGK

SEQ ID NO: 91 - protein, 16 aa, Homo sapiens
LIAPVAEEEATVPNNK

SEQ ID NO: 92 - protein, 10 aa, Homo sapiens
SADTLWDIQK

SEQ ID NO: 93 - protein, 8 aa, Homo sapiens
FIIPQIVK

SEQ ID NO: 94 - protein, 15 aa, Homo sapiens
SLADELALVDVLEDK

SEQ ID NO: 95 - protein, 9 aa, Homo sapiens
DYSVTANSK

SEQ ID NO: 96 - protein, 10 aa, Homo sapiens
LKDDEVAQLK

SEQ ID NO: 97 - protein, 9 aa, Homo sapiens
GLTSVINQK

SEQ ID NO: 98 - protein, 9 aa, Homo sapiens
IVVVTAGVR

SEQ ID NO: 99 - protein, 11 aa, Homo sapiens
MVVESAYEVIK

SEQ ID NO: 100 - protein, 12 aa, Homo sapiens
VIGSGCNLDSAR

SEQ ID NO: 101 - protein, 20 aa, Homo sapiens
GMYGIENEVFLSLPCILNAR

SEQ ID NO: 102 - protein, 9 aa, Homo sapiens
IHPVSTMVK

SEQ ID NO: 103 - protein, 34 aa, Homo sapiens
SEDAGGAEGETPHMLLRPHVFMPEVTPDMDYLPR

SEQ ID NO: 104 - protein, 36 aa, Homo sapiens
IFSEDDDYIDIVDSLSVSPTDSDVSAGNILQLFHGK

(19) United States
(12) Patent Application Publication
(10) Pub. No.: US 2025/0174303 A1
(43) Pub. Date: May 29, 2025

(54) PEPTIDE CENTRIC ANALYSES

(71) Applicant: Seer, Inc., Redwood City, CA (US)

(72) Inventors: Margaret Donovan, San Franciso, CA (US); Yingxiang Huang, San Leandro, CA (US); Jian Wang, San Diego, CA (US); Sangtae Kim, San Diego, CA (US); John Blume, Bellingham, WA (US); Asim Siddiqui, San Francisco, CA (US); Daniel Hornburg, Foster City, CA (US); Shadi Roshdiferdosi, San Carlos, CA (US); Mahdi Zamanighomi, Belmont, MA (US); Harendra Guturu, Oakland, CA (US); Alexey Stukalov, Redwood City, CA (US)

(21) Appl. No.: 18/726,613

(22) PCT Filed: Jan. 6, 2023
(86) PCT No.: PCT/US2023/060271

§ 371 Date: Jul. 3, 2024

Related U.S. Application Data
(60) Provisional application No. 63/297,510, filed on Jan. 7, 2022.
(60) Provisional application No. 63/306,967, filed on Feb. 4, 2022.
(60) Provisional application No. 63/348,668, filed on Jun. 3, 2022.

(51) Int. Cl. G16B 25/10; G16B 20/20

(52) U.S. Cl. CPC G16B 25/10; G16B 20/20

(57) ABSTRACT

The present disclosure describes a method for assaying a biological sample. In some cases, the method comprises assaying a set of nucleic acids from the biological sample to obtain genotypic information of the biological sample. In some cases, the method comprises generating, based at least in part on the genotypic information, a set of expressible proteoforms that can be expressed from the set of nucleic acids. In some cases, the method comprises assaying a set of polyamino acids from the biological sample to generate proteomic information of the biological sample, wherein the proteomic information comprises a set of identifications for the set of polyamino acids. In some cases, the method comprises mapping the set of identifications to the set of expressible proteoforms, thereby determining a set of expressed proteoforms in the biological sample.

### CROSS-REFERENCE

This application claims benefit of U.S. Provisional Application No. 63/348,668, filed on Jun. 3, 2022, U.S. Provisional Application No. 63/306,967, filed on Feb. 4, 2022, and U.S. Provisional Application No. 63/297,510, filed on Jan. 7, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Jan. 6, 2023, is named 53344-751-601_SL.xml and is 90,657 bytes in size.

### BACKGROUND

Biological samples contain a wide variety of proteins and nucleic acids. Compositions and methods are needed for elucidating the presence and concentration of proteins and nucleic acids as well as any correlations between proteins and nucleic acids that may be indicative of a biological state.

### SUMMARY

In some aspects, the present disclosure describes a method for assaying a biological sample, comprising: (a) assaying a set of nucleic acids from the biological sample to obtain genotypic information of the biological sample; (b) generating, based at least in part on the genotypic information, a set of expressible proteoforms that can be expressed from the set of nucleic acids; (c) assaying a set of polyamino acids from the biological sample to generate proteomic information of the biological sample, wherein the proteomic information comprises a set of identifications for the set of polyamino acids; and (d) mapping the set of identifications to the set of expressible proteoforms, thereby determining a set of expressed proteoforms in the biological sample.

In some cases, the set of nucleic acids is an exome of the biological sample and the genotypic information comprises an exome sequence of the biological sample.

In some cases, the set of proteoforms comprise peptide variants, peptidoforms, protein variants, or combinations thereof. In some cases, the set of proteoforms comprise splicing variants, allelic variants, post-translation modification variants, or any combination thereof.

In some cases, the set of polyamino acids comprise a set of peptide fragments derived from a set of proteins expressed in the biological sample. In some cases, the set of peptide fragments are derived by enzymatic digestion. In some cases, the set of peptide fragments are derived by trypsinization. In some cases, the set of peptide fragments comprise tryptic peptide fragments, semi-tryptic peptide fragments, or both. In some cases, the set of peptide fragments comprise tryptic peptide fragments, semi-tryptic peptide fragments, or both.

In some cases, the method further comprises filtering the set of expressible proteoforms for a proteoform type. In some cases, the filtering is based on a statistical significance value that an expressible proteoform in the set of expressible proteoforms comprises the proteoform type. In some cases, the proteoform type is a splicing variant. In some cases, the statistical significance value is based on a probability that a sequence of the expressible proteoform comprises a reordered amino acid sequence of another expressible proteoform from the same protein group as the expressible proteoform. In some cases, the statistical significance value is based on a probability that a sequence of the expressible proteoform comprises a subsequence of an amino acid sequence of another expressible proteoform from the same protein group as the expressible proteoform. In some cases, the proteoform type is an allelic variant. In some cases, the statistical significance value is based on a probability that a sequence of the expressible proteoform comprises an amino acid substitution in an amino acid sequence of another expressible proteoform from the same protein group. In some cases, the proteoform type is a post-translational cleavage variant. In some cases, the statistical significance value is based on a probability that peptide fragments of the expressible proteoform is localized on one terminus of another expressible proteoform from the same protein group. In some cases, the proteoform type is a phosphorylated variant. In some cases, the statistical significance value is based on a probability that a sequence of the expressible proteoform comprises a phosphorylated amino acid.

In some cases, the set of polyamino acids comprise a set of proteins expressed in the biological sample. In some cases, a polyamino acid may include proteins. In some cases, a polyamino acid may include peptides. In some cases, a polyamino acid may include polypeptides. In some cases, polyamino acids may include polypeptide strands synthesized by a cell and secreted or otherwise found in a biofluid of a subject.

In some cases, the proteomic information comprises a set of peptide intensities detected from assaying the set of polyamino acids.

In some cases, the set of identifications comprises protein group identifications for the set of polyamino acids. In some cases, the set of identifications comprises amino acid sequences for the set of polyamino acids. In some cases, the set of identifications comprises post-translational modifications for the set of polyamino acids.

In some cases, the mapping comprises matching an identification in the set of identifications to an expressible proteoform in the set of expressible proteoforms, thereby determining that the matched expressible proteoform is an expressed proteoform in the biological sample.

In some cases, the method further comprises associating the set of expressed proteoforms with a biological state of the biological sample. In some cases, the associating is based at least partially on the expression levels of each proteoform in the set of expressed proteoforms.

In some cases, the method further comprises associating the genotypic information with the biological state of the biological sample.

In some cases, the set of polyamino acids are derived from the biological sample using at least one untargeted assay. In some cases, the set of polyamino acids comprise a dynamic range of at least 5, 6, 7, 8, 9, 10, 11, or 12 orders of magnitude in the biological sample. In some cases, the set of polyamino acids comprise a dynamic range of at most 6, 7, 8, 9, 10, 11, 12, 15, 20 or 25 orders of magnitude in the biological sample.

In some cases, the at least one untargeted assay comprises: (a) providing a plurality of surface regions comprising a plurality of surface types; (b) contacting the biological sample with the plurality of surface regions to yield a set of adsorbed biomolecules on the plurality of surface regions; and (c) desorbing, from the plurality of surface regions, at least a portion of the set of adsorbed biomolecules to yield the set of polyamino acids.

In some cases, the plurality of surface regions are disposed on a single continuous surface. In some cases, the plurality of surface regions are disposed on a plurality of discrete surfaces. In some cases, the plurality of discrete surfaces are surfaces of a plurality of particles.

In some cases, the at least one untargeted assay has a false discovery rate of at most about 5%, 4%, 3%, 2%, 1%, 0.9%, 0.8%, 0.7%, 0.6%, 0.5%, 0.4%, 0.3%, 0.2%, or 0.1%.

In some cases, the set of expressed proteoforms comprises at least about 10, 20, 30, 40, 50, 60, 70, 80, 90, 100, 200, 300, 400, 500, 600, 700, 800, 900, or 1,000 expressed proteoforms.

In some aspects, the present disclosure describes a method for assaying a biological sample, comprising: (a) assaying a set of polyamino acids from the biological sample to generate proteomic information of the biological sample, wherein the proteomic information comprises a set of identifications for the set of polyamino acids; (b) assaying a set of nucleic acids from the biological sample to obtain genotypic information of the biological sample, wherein the genotypic information comprises one or more nucleic acid sequences; and (c) determining expression levels of one or more regions in the one or more nucleic acid sequences, based at least partially on the set of identifications.

In some cases, the set of polyamino acids comprises a set of peptide fragments derived from a set of proteins expressed in the biological sample.

In some cases, the set of identifications comprises protein group identifications or amino acid sequences for the set of polyamino acids.

In some cases, the set of nucleic acids is an exome of the biological sample and the genotypic information comprises an exome sequence of the biological sample.

In some cases, the one or more regions are one or more exons in the exome sequence.

In some cases, the set of polyamino acids are derived from the biological sample using at least one untargeted assay. In some cases, the set of polyamino acids comprise a dynamic range of at least 5, 6, 7, 8, 9, 10, 11, or 12 orders of magnitude in the biological sample. In some cases, the set of polyamino acids comprise a dynamic range of at most 6, 7, 8, 9, 10, 11, 12, 15, 20 or 25 orders of magnitude in the biological sample.

In some cases, the at least one untargeted assay comprises: (a) providing a plurality of surface regions comprising a plurality of surface types; (b) contacting the biological sample with the plurality of surface regions to yield a set of adsorbed biomolecules on the plurality of surface regions; and (c) desorbing, from the plurality of surface regions, at least a portion of the set of adsorbed biomolecules to yield the set of polyamino acids.

In some cases, the plurality of surface regions are disposed on a single continuous surface. In some cases, the plurality of surface regions are disposed on a plurality of discrete surfaces. In some cases, the plurality of discrete surfaces are surfaces of a plurality of particles.

In some cases, the determining further comprises identifying one or more base positions in the one or more nucleic acid sequences that covaries with at least one element in the proteomic information. In some cases, the one or more base positions comprise a single nucleotide polymorphism. In some cases, the at least one element comprises a polyamino acid identification in the set of polyamino acid identifications and a polyamino acid intensity measured using the untargeted assay. In some cases, the determining further comprises filtering the one or more base positions when a statistical significance value for the one or more base pair positions is less than a threshold statistical significance value. In some cases, the statistical significance value is a p-value. In some cases, the threshold statistical significance value is 1e⁻⁵.

In some cases, the determining further comprises filtering the one or more base positions when a false discovery rate for the one or more base pair positions is less than a threshold false discovery rate. In some cases, the false discovery rate is determined by: (a) shuffling the proteomic data to generate a shuffled proteomic data; (b) identifying one or more decoy base positions in a shuffled proteomic data that covaries with at least one element in the proteomic information; and (c) normalizing the number of the one or more decoy base positions by the number of the one or more base positions.

In some cases, the method further comprises classifying the one or more base positions as a cis-pQTL or a trans-pQTL based on a distance between the one or more base positions and a gene that encodes a polyamino acid comprising the polyamino acid identification. In some cases, the one or more base positions are classified as a cis-pQTL when the distance is less than 1 megabase pairs (Mbp) of a transcription start site of the gene. In some cases, the one or more regions in the one or more nucleic acid sequences comprises the gene that encodes a polyamino acid comprising the polyamino acid identification.

In some aspects, the present disclosure describes a method for identifying a differentially expressed polyamino acid, comprising: (a) obtaining a plurality of polyamino acids from a plurality of biological samples, wherein the plurality of biological samples are differential in at least one clinically relevant dimension; (b) assaying the plurality of polyamino acids, using at least one untargeted assay, to generate a plurality of identifications for the plurality of polyamino acids; and (c) identifying at least one polyamino acid in the plurality of polyamino acids that is differentially expressed or abundant in the at least one clinically relevant dimension. In some cases, the plurality of polyamino acids comprises one or more peptide fragments derived from proteins expressed in the plurality of biological samples. In some cases, the set of peptide fragments are derived by enzymatic digestion. In some cases, the set of peptide fragments are derived by trypsinization. In some cases, the set of peptide fragments comprise tryptic peptide fragments, semi-tryptic peptide fragments, or both. In some cases, the set of peptide fragments comprise tryptic peptide fragments, semi-tryptic peptide fragments, or both.

In some cases, the method further comprises filtering the set of expressible proteoforms for a proteoform type. In some cases, the filtering is based on a statistical significance value that an expressible proteoform in the set of expressible proteoforms comprises the proteoform type. In some cases, the proteoform type is a splicing variant. In some cases, the statistical significance value is based on a probability that a sequence of the expressible proteoform comprises a reordered amino acid sequence of another expressible proteoform from the same protein group as the expressible proteoform. In some cases, the statistical significance value is based on a probability that a sequence of the expressible proteoform comprises a subsequence of an amino acid sequence of another expressible proteoform from the same protein group as the expressible proteoform. In some cases, the proteoform type is an allelic variant. In some cases, the statistical significance value is based on a probability that a sequence of the expressible proteoform comprises an amino acid substitution in an amino acid sequence of another expressible proteoform from the same protein group. In some cases, the proteoform type is a post-translational cleavage variant. In some cases, the statistical significance value is based on a probability that peptide fragments of the expressible proteoform is localized on one terminus of another expressible proteoform from the same protein group. In some cases, the proteoform type is a phosphorylated variant. In some cases, the statistical significance value is based on a probability that a sequence of the expressible proteoform comprises a phosphorylated amino acid. In some cases, the probability may be based on a peptide search algorithm or a protein grouping algorithm. In some cases, the probability may be based on a mass spectrogram of the proteoform or a fragment thereof.

In some case, the at least one identified polyamino acid is differentially expressed or abundant relative to a second polyamino acid in the plurality of polyamino acids, wherein the at least one identified polyamino acid and the second polyamino acid are derived from the same protein or protein group expressed in the plurality of biological samples.

In some cases, the at least one clinically relevant dimension is a disease state. In some cases, the disease state is a presence of cancer or an absence of cancer. In some cases, the disease state is a stage of cancer.

In some cases, the plurality of polyamino acids are peptide fragments derived from proteins expressed in the plurality of biological samples. In some cases, the set of polyamino acids comprise a dynamic range of at least 5, 6, 7, 8, 9, 10, 11, or 12 orders of magnitude in the biological sample.

In some cases, the at least one untargeted assay comprises: (a) providing a plurality of surface regions comprising a plurality of surface types; (b) contacting the biological sample with the plurality of surface regions to yield a set of adsorbed biomolecules on the plurality of surface regions; and (c) desorbing, from the plurality of surface regions, at least a portion of the set of adsorbed biomolecules to yield the set of polyamino acids.

In some cases, the plurality of surface regions are disposed on a single continuous surface. In some cases, the plurality of surface regions are disposed on a plurality of discrete surfaces. In some cases, the plurality of discrete surfaces are surfaces of a plurality of particles. In some cases, the plurality of particles are dispersed in solution. In some cases, the plurality of particles are provided as a suspension in solution.

In some aspects, the present disclosure describes a method for assaying a biological sample, comprising: (a) assaying a set of peptides from a plurality of biological samples to obtain a set of peptide identifications; (b) identifying a set of protein groups based at least in part on the set of peptide identifications; (c) determining, for a given protein group in the set of protein groups, a set of correlated peptides that are correlated in abundance across the plurality of biological samples; and (d) mapping the set of correlated peptides to a set of expressible proteoforms, thereby identifying at least one proteoform common in the plurality of biological samples.

In some aspects, the present disclosure provides a method for assaying a biological sample, comprising: (a) assaying a set of peptides from the biological sample using spectral data to generate proteomic information of the biological sample, wherein the proteomic information comprises a set of identifications for the set of peptides; (b) identifying a set of protein groups based at least in part on the spectral data of the set of peptides; (c) identifying one or more sets of peptides that are correlated in abundance for a given protein group in the set of protein groups; and (d) mapping the set of peptides to a database of human genes with isoform information, thereby determining a set of proteoforms that result in the set of peptides. In some embodiments, the spectral data comprises mass spectrometry data. In some embodiments, the identifying the one or more sets of peptides comprises computing a correlation of abundances of each peptide across the biological sample. In some embodiments, the identifying the one or more sets of peptides comprises computing a correlation of abundances of each peptide across a plurality of biological samples or clustering based on peptides' correlations. In some embodiments, the method further comprises, subsequent to (c), identifying a first set of peptides that are correlated in abundance; further comprising identifying a second set of peptides that are correlated in abundance; and further comprising applying a filtering step to confirm that the set of peptides are distinct from each other. In some embodiments, the method further comprises identifying more than two sets of peptides that are correlated in abundance, and applying a filtering step to confirm that the more than two sets of peptides are distinct from each other. In some embodiments, the biological sample comprises a plasma sample derived from subjects afflicted with a non-small cell lung cancer. In some embodiments, an identified proteoform is associated with a disease. In some embodiments, the set of proteoforms comprise peptide variants, protein variants, or both. In some embodiments, the set of proteoforms comprise splicing variants, allelic variants, post-translation modification variants, or any combination thereof. In some embodiments, the database of human genes comprises an ENSEMBL database with isoform information.

In some aspects, the present disclosure provides a computer-implemented method for assaying a biological sample, comprising: retrieving genotypic information associated with the biological sample from a database; generating, based at least in part on the genotypic information, a set of expressible proteoforms that can be expressed from the set of nucleic acids; retrieving assay data for a set of polyamino acids from the biological sample from a database; generating proteomic information of the biological sample using the assay data, wherein the proteomic information comprises a set of identifications for the set of polyamino acids; and mapping the set of identifications to the set of expressible proteoforms, thereby determining a set of expressed proteoforms in the biological sample. In some embodiments, the genotypic information comprises whole genome sequence data associated with the biological sample. In some embodiments, the genotypic information comprises exome sequence data, transcriptome sequence data, epigenome sequence data, or any combination thereof associated with the biological sample. In some embodiments, the proteomic information further comprises abundance data for the set of polyamino acids.

In some aspects, the present disclosure provides a computer-implemented method for assaying a biological sample, comprising: retrieving genotypic information associated with the biological sample from a database, wherein the genotypic information comprises one or more nucleic acid sequences; retrieving assay data for a set of polyamino acids from the biological sample from a database; generating proteomic information of the biological sample using the assay data, wherein the proteomic information comprises a set of identifications for the set of polyamino acids; determining expression levels of one or more regions in the one or more nucleic acid sequences, based at least partially on the set of identifications. In some embodiments, the genotypic information comprises whole genome sequence data associated with the biological sample. In some embodiments, the genotypic information comprises exome sequence data, transcriptome sequence data, epigenome sequence data, or any combination thereof associated with the biological sample.

In some embodiments, the assaying comprises mass spectrometry or protein sequencing.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1H show proteome analysis of healthy and NSCLC subjects using a 5 NP plasma workflow, in accordance with some embodiments.

FIG. 1A shows an overview of a proteoform identification study. Plasma samples were collected from healthy (medium stippled), early non-small cell lung cancer (NSCLC; no stipple), late NSCLC (lightly stippled), and co-morbid (heavily stippled) subjects (Sample Collection). The plasma proteomes were analyzed for each of these subjects, which included protein extraction, protein discovery using the NP-based PROTEOGRAPH™ platform, then DIA and DDA protein/peptide identification and quantification using LC-MS/MS and search algorithms (Proteome Analysis). Proteoforms were then identified using two strategies: 1) Discordant peptide intensity search, which included examining peptide mappings to known protein coding isoforms and using differential abundance to discover protein isoforms; and 2) Proteogenomic search, which included using genotype information (whole exome sequencing; WES) to perform personalized database searches and identify protein variants not captured in standard protein databases (Proteoform Identification). Together, these identified proteoforms represent an expanded plasma proteome database not captured in standard MS-based or targeted proteomic studies (Expanded Proteome). Peptide sequences GDTSTIYTNCWVTGWGFSK (SEQ ID NO: 3) and GDTSTIYTNCWVTR (SEQ ID NO: 4) are illustrated as protein variant 1 and protein variant 2, respectively. See Table 1 for list of all sequences.

FIG. 1B shows a dot plot representing the number of protein groups (y-axis) identified across study samples (x-axis), ranging from protein groups identified in one or more samples (“any”) to proteins identified in 100% of samples (“100”). 25%, 50%, and 75% of samples are highlighted with grey dashed lines.

FIG. 1C shows a bar plot showing the number of peptides detected per protein group (x-axis) and the number of protein groups we observe for each bin (y-axis). The median (thick dashed line) and mean (thin dashed line) are shown.

FIG. 1D shows a plot of protein groups matched to a reference database (HPPP) as a distribution by the rank order of published concentrations (x-axis) and by the log₁₀ published concentration (ng/ml; y-axis). The first, second, and third quantiles are highlighted with grey dashed lines. For each quantile, the number and fraction of protein groups matching the reference database are reported.

FIG. 1E shows barplots showing the number of peptides and proteins groups retained after filtering to those present in at least 50% of subjects from either heathy or early NSCLC.

FIG. 1F shows barplots showing the number of differentially abundant (DA): 1) protein groups, with collapsed abundances using MaxLFQ; 2) protein groups across NPs (i.e., DA independently across NPs); and 3) peptides across NPs.

FIG. 1G shows volcano plot showing the statistical significance (adjusted p-value; y-axis) and fold change (x-axis) from calculating the differential abundance of protein groups across NPs between healthy and early NSCLC subjects. Protein groups with a log₂ (Fold Change) greater or less than 1.0 and adjusted p-value<0.05 are highlighted, where protein groups with increased abundance in early NSCLC subjects are shown in light gray (top right rectangle) and protein groups with increased abundance in healthy subjects are shown in dark gray (top left rectangle). Proteins with known roles in cancer and immune response (ITIH2, CRP, S100A9, S100A8, ANTXR2, and ANTXR1) are highlighted with various shapes.

FIG. 1H shows volcano plot showing the statistical significance (adjusted p-value; y-axis) and fold change (x-axis) from calculating the differential abundance of peptides across NPs between healthy and early NSCLC subjects. Peptides with a log₂ (Fold Change) greater or less than 1.0 and adjusted p-value<0.05 are highlighted, where peptides with increased abundance in early NSCLC subjects are shown in light gray (top right rectangle) and peptides with increased abundance in healthy subjects are shown in dark gray (top left rectangle). Peptides mapping to proteins with known roles in cancer and immune response (ITIH2, CRP, S100A9, S100A8, ANTXR2, and ANTXR1) are highlighted with various shapes.

FIGS. 2A-2H show identification of four proteoforms, including BMP1, in 141 healthy and early NSCLC subjects using a discordant peptide intensity search.

FIG. 2A shows a cartoon describing the discordant peptide intensity search strategy. DA was calculated across peptides between healthy (stippled) and early NSCLC (no stipple). Protein groups with at least one peptide significantly over-expressed (triple asterisks) in healthy subjects (left arrow) and at least one peptide over-expressed in early NSCLC subjects (right arrow) were identified as having putative proteoforms. Mapping the peptides to the gene structure, potential exon usage and segments were inferred, suggesting the detection of more than one protein isoform.

FIG. 2B shows a barplot showing four proteins in which multiple protein isoforms were potentially captured: BMP1, C4A, CIR, and LDHB and their associated Open Target Score for lung carcinoma.

FIG. 2C shows a plot showing the four proteins with putative proteoforms matched to a reference database (HPPP) plotted as a distribution by the rank order of published concentrations (x-axis) and by the log₁₀ published concentration (ng/ml; y-axis).

FIG. 2D shows a box plot showing the log₁₀ median normalized intensities of BMP1 in early NSCLC subjects (no stipple) and in healthy subjects (stippled) with collapsed abundances across NPs. P-values, calculated using a Wilcoxon test, are shown.

FIG. 2E shows a box plot showing the log₁₀ median normalized intensities of BMP1 in early NSCLC subjects (no stipple) and in healthy subjects (stippled) in NP, NP-C. P-values, calculated using a Wilcoxon test, are shown.

FIG. 2F shows a series of boxplots showing the log₁₀ median normalized intensities of seven peptides mapping BMP1 in early NSCLC (no stipple) and healthy subjects (stippled). Peptides that are over-expressed in healthy subjects are indicated with an arrow (thin outline) and in early NSCLC are indicated with an arrow (thick outline). Peptides that are significantly DA are indicated with a triple asterisk. P-values, calculated using a Wilcoxon test and adjusted, are shown.

FIG. 2G shows a heatmap showing the Pearson correlation of the seven BMP1 peptide abundances, where low correlation is indicated top right and bottom left quadrants, and high correlation is indicated in top left and bottom right quadrants. Correlation values were clustered using hierarchical clustering. Peptides are annotated by the direction of DA, including over-expressed in healthy subjects are highlighted in dark gray and early NSCLC are highlighted in light gray.

FIG. 2H shows gene structure plots of four known BMP1 protein coding transcripts (i.e., isoforms) with the seven BMP1 peptides mapped to genomic region. Peptides spanning intronic regions are indicated with a horizontal line. Peptides 1 and 2, corresponding to being over-expressed early NSCLC, are boxed in thick dashed lines, creating one segment. Peptides 37, corresponding to being over-expressed healthy, are boxed in thin dashed lines, creating a second segment. Segment 2 appears to correspond to the shorter isoform 1, whereas segment 2 appears to correspond to the longer isoforms 2-4.

FIGS. 3A-3E show identification of 422 protein variants using a custom proteogenomic search.

FIG. 3A shows a cartoon describing a proteogenomic search to identify protein variants. Exomes of 29 subjects were sequenced to 100×, followed by genomic variant calling. The genomic sequence, including identified variants, were translated to protein sequence and digested in silico. These custom sequences were then combined with a reference sequence database to generate a personalized database. Personalized databases were generated for each of the 29 subjects and used to analyze the MS DDA data and search for variant peptides. Shown are nucleic acid sequences SEQ ID NO: 5 and SEQ ID NO: 6, and amino acid sequences SEQ ID NOs: 7-9.

FIG. 3B shows bar plots showing the number of protein variants (y-axis) identified across 29 subjects (x-axis), including subjects with co-morbidities (stippled), healthy (stippled), early NSCLC (no stipple), and late NSCLC (lightly stippled).

FIG. 3C shows a distribution of the number of variants (y-axis) across alternative allele frequencies (x-axis) in the 1k Genome Project and in the 422 protein variants.

FIGS. 3D-3E shows tandem mass spectra of peptides HPLKPDNQPFPQSVSESCPGK (SEQ ID NO: 1) and HPLKPDIQPFPQSVSESCPGK (SEQ ID NO: 2) arising from a heterozygous variant, where the alternative allele causes a single amino acid variant (SAAV; N→I). Both the reference peptide (FIG. 3D) and alternative (variant) peptide (FIG. 3E) are observed in the MS data.

FIGS. 4A-4E shows identification of C4A proteoform in in 141 healthy and early NSCLC subjects using a discordant peptide intensity search.

FIG. 4A shows a box plot showing the log₁₀ median normalized intensities of C4A in early NSCLC subjects (no stipple) and in healthy subjects (stippled) with collapsed abundances across NPs. P-values, calculated using a Wilcoxon test, are shown.

FIG. 4B shows a box plot showing the log₁₀ median normalized intensities of C4A in early NSCLC subjects (no stipple) and in healthy subjects (stippled) in NP, NP-A. P-values, calculated using a Wilcoxon test, are shown.

FIG. 4C shows a heatmap showing the Pearson correlation of the seven C4A peptide abundances, where low correlation is indicated in top right and bottom left quadrants and high correlation is indicated in top left and bottom right quadrants. Correlation values were clustered using hierarchical clustering. Peptides are annotated by the direction of DE, including over-expressed in healthy subjects are in the major right branch and early NSCLC are in the major left branch. Shown are SEQ ID NOs: 10-73.

FIG. 4D shows a series of boxplots showing the log₁₀ median normalized intensities of 64 peptides mapping C4A in early NSCLC (no stipple) and healthy subjects (stippled). Peptides that are over-expressed in healthy subjects are indicated with a thinly outlined arrow and in early NSCLC are indicated with a thick outlined arrow. Peptides that are significantly DE are indicated with a triple asterisk. P-values, calculated using a Wilcoxon test and adjusted, are shown.

FIG. 4E shows a gene structure plots of 64 known C4A protein coding transcripts (i.e., isoforms) with the 64 C4A peptides mapped to genomic region. Peptides spanning intronic regions are indicated with a horizontal line. Peptides 1-39 (except peptide 32), corresponding to being over-expressed in healthy subjects, are boxed in thin dashed lines, creating one segment. Peptides 40-53, corresponding to being over-expressed early NSCLC, are boxed in thick dashed lines, creating a second segment. Peptides 54-63 (except peptide 64), corresponding to being over-expressed in healthy subjects, are boxed in thin dashed lines, creating a third segment. Segment patterns do not appear to correspond to any known protein isoforms, potentially indicating a novel isoform.

FIG. 5A-5E shows identification of CIR proteoform in in 141 healthy and early NSCLC subjects using a discordant peptide intensity search.

FIG. 5A shows a box plot showing the log₁₀ median normalized intensities of CIR in early NSCLC subjects (no stipple) and in healthy subjects (stippled) with collapsed abundances across NPs. P-values, calculated using a Wilcoxon test, are shown.

FIG. 5B shows a box plot showing the log₁₀ median normalized intensities of CIR in early NSCLC subjects (no stipple) and in healthy subjects (stippled) in NP, NP-B. P-values, calculated using a Wilcoxon test, are shown.

FIG. 5C shows a heatmap showing the Pearson correlation of the seven CIR peptide abundances, where high correlation is indicated in the top left quadrant and low correlation is indicated everywhere else. Correlation values were clustered using hierarchical clustering. Peptides are annotated by the direction of DE, including over-expressed in healthy subjects are highlighted in dark gray and early NSCLC are highlighted in light gray. Shown are SEQ ID NOs: 74-90.

FIG. 5D shows a series of boxplots showing the logo median normalized intensities of 17 peptides mapping CIR in early NSCLC (no stipple) and healthy subjects (stippled). Peptides that are over-expressed in healthy subjects are indicated with a thinly outlined arrow and in early NSCLC are indicated with a thick outlined arrow. Peptides that are significantly DE are indicated with a triple asterisk. P-values, calculated using a Wilcoxon test and adjusted, are shown.

FIG. 5E shows a gene structure plots of 17 known CIR protein coding transcripts (i.e., isoforms) with the 17 CIR peptides mapped to genomic region. Peptides spanning intronic regions are indicated with a horizontal line. Peptides corresponding to being over-expressed in healthy subjects are boxed in thin dashed lines. Peptides corresponding to being over-expressed early NSCLC are boxed in thick dashed lines.

FIGS. 6A-6E shows identification of LDHB proteoform in in 141 healthy and early NSCLC subjects using a discordant peptide intensity search.

FIG. 6A shows a box plot showing the log₁₀ median normalized intensities of LDHB in early NSCLC subjects (no stipple) and in healthy subjects (stippled) with collapsed abundances across NPs. P-values, calculated using a Wilcoxon test, are shown.

FIG. 6B shows a box plot showing the log₁₀ median normalized intensities of LDHB in early NSCLC subjects (no stipple) and in healthy subjects (stippled) in NP, NP-A. P-values, calculated using a Wilcoxon test, are shown.

FIG. 6C shows a heatmap showing the Pearson correlation of the seven LDHB peptide abundances, where and high correlation is indicated in the top left quadrant and low correlation is indicated everywhere else. Correlation values were clustered using hierarchical clustering. Peptides are annotated by the direction of DE, including over-expressed in healthy subjects are highlighted in dark gray and early NSCLC are highlighted in light gray. Shown are SEQ ID NOs: 91-102.

FIG. 6D shows a series of boxplots showing the log₁₀ median normalized intensities of 12 peptides mapping LDHB in early NSCLC (no stipple) and healthy subjects (stipple). Peptides that are over-expressed in healthy subjects are indicated with a thin outlined arrow and in early NSCLC are indicated with a thick outlined arrow. Peptides that are significantly DE are indicated with a triple asterisk. P-values, calculated using a Wilcoxon test and adjusted, are shown.

FIG. 6E shows a gene structure plots of 12 known LDHB protein coding transcripts (i.e., isoforms) with the 12 LDHB peptides mapped to genomic region. Peptides spanning intronic regions are indicated with a horizontal line. Peptides corresponding to being over-expressed in healthy subjects are boxed in thin dashed lines. Peptides corresponding to being over-expressed early NSCLC are boxed in thick dashed lines.

FIG. 7 shows a computer system that is programmed or otherwise configured to implement methods provided herein.

FIG. 8A shows a photograph of labeled tubes, in accordance with some embodiments.

FIG. 8B shows photographs schematically illustrating a method for collecting a biological sample, in accordance with some embodiments.

FIG. 8C shows a photograph illustrating a method for collecting a biological sample, in accordance with some embodiments.

FIGS. 9A-9B show data obtained by the identification of dependent peptides in 29 healthy, early NSCLC, late NSCLC, and comorbid subjects using MaxQuant's dependent peptide search, in accordance with some embodiments. FIG. 9A shows bar plots depicting the median number of features at the level of MS/MS, MS1, dependent peptide IDs, peptide IDs, and protein group IDs for each individual DDA run, in accordance with some embodiments. Peptide, protein and dependent peptide results are filtered for 1% FDR. The error bars show the standard deviation across subjects and NPs. Top panel in FIG. 9B shows a bubble chart depicting the different modifications identified in the dataset, in accordance with some embodiments. The size of each bubble is relative to number of peptides identified with that modification. The x-axis shows the median mass offset for each modification and y-axis shows the number of unique modifications in each 10 Da mass offset bin. Bottom panel in FIG. 9C shows a density plot showing the distribution of mass differences associated with dependent peptides in the data set.

FIGS. 10A-10B show identification of phosphorylated peptides in NSCLC samples, in accordance with some embodiments. Box plots show two examples of identified phosphorylated peptides that display differentially expressed pattern between the different sample groups. The labile-phospho workflow from Fragpipe (version 17.1) was used to identify phosphorylation sites and results was filtered at 1% peptide-level FDR. The ratio between the abundance of the unmodified peptide and the phosphorylated version of the same peptide was computed. If such modified/unmodified peptides pairs were observed in samples from multiple NP fraction, the median ratio across multiple NPs was used. To detect differentially expressed peptides, the Wilcoxson rank sum test was applied to the ratios and filtered at a p-value of 0.05. Shown in FIG. 10A is SEQ ID NO: 103, and shown in FIG. 10B is SEQ ID NO: 104.

FIG. 11 illustrates a method for detecting proteoforms from proteomes, in accordance with some embodiments.

FIG. 12 schematically illustrates a calculation for pQTL association, in accordance with some embodiments.

FIG. 13 shows different conditions for forming protein corona with nanoparticles, in accordance with some embodiments.

FIG. 14 shows the number of inferred proteoforms based on peptide distinct peptide correlation profiles across corona dynamics (time and P/NP ratio) for nanoparticles, in accordance with some embodiments.

FIGS. 15A-15B show examples of peptide maps that indicate different protein isoforms, in accordance with some embodiments.

FIGS. 16A-16B show corona dynamic profiles of different protein isoforms of the same protein group, in accordance with some embodiments.

### DETAILED DESCRIPTION

Comprehensive assessment of the human proteome is challenging due to multiple forms of a protein arising from alternative splicing, allelic variation, and protein modifications. Characterization of the multiple forms of a protein, or proteoforms, produced by alternative splicing, allelic variation, and protein modifications, can serve distinct functions, can expand understanding of the molecular mechanisms underlying health and disease. As proteoforms can act as functional links between genotype and phenotype, proteoform-level knowledge is important for understanding the molecular mechanisms in biology. Some challenges in the identification of proteoforms are: (i) obtaining protein coverage at amino acid resolution, and (ii) obtaining protein coverage scalably and deeply without lengthy workflows.

In some cases, multiple isoforms of a single protein, or proteoforms, can arise due to alternative splicing (i.e., protein isoforms), allelic variation (i.e., protein variants), and post translation modifications (PTMs). Proteoforms can play key and distinct roles in biological mechanisms, including impacting complex traits and disease. Genetic variation can give rise to changes to the genome that can be functionally neutral, however some genetic variants, such as non-synonymous variants resulting in the alteration of an amino acid sequence (e.g., those that lead to protein variants), can drastically impact phenotype. In some cases, rare variants of proteoforms may be highly enriched for pathogenicity (i.e., are much more likely to be deleterious and to have a large effect in common and rare disease) and common variants of proteoforms may be either benign or to have a small effect in disease. In a population, rare genetic variants may vastly outnumber common variants. A proteome may harbor a large fraction of putatively physiologically relevant rare proteoforms. The putatively physiologically relevant rare proteoforms can be difficult to access with protein affinity-based targeted methods, because there are estimated to be over 1 million distinct proteoforms in a given cell type. Thus, in targeted methodologies such as immunoassays, designing a panel comprising all these potential proteoforms can be immensely challenging. We show untargeted approaches that identify both protein isoforms and protein variants provide a deeper, more nuanced assessment of human proteomes, supporting enhanced understanding of human health and disease. The present disclosure describes an untargeted approach for analyzing a proteome that can identify both protein isoforms and protein variants to provide a more nuanced assessment of proteomes.

Important advances in characterizing the proteomic landscape of lung cancers such as non-small cell lung cancer (NSCLC) and squamous cell lung cancer have identified important protein biomarkers. However, relatively few proteoforms relevant to lung cancer have been identified. Readout technologies such as high resolution quantitative mass spectrometry (MS) can be employed to infer and to quantify peptides and proteins with high confidence (e.g., <1% false discovery rate (FDR)). However, large-scale LC-MS/MS-based proteomics studies can be challenging due to lengthy workflows required to achieve deep (e.g., broad detection of proteins across the dynamic range, from high to low abundance proteins) and unbiased (e.g., hypothesis-free detection) sampling of clinically relevant biospecimens with large dynamic ranges of protein abundances, such as blood plasma. While LC-MS and LC-MS/MS methodologies may offer the capability to infer proteoforms, peptide identification in LC-MS/MS-based proteomic data may rely on protein databases, such as UniProt, which may exclude proteoforms that may be present in an individual's proteome.

The present disclosure discloses methods and systems for performing fast, scalable, deep, and unbiased plasma proteomics. In some cases, the methods and systems may be used to identify known and/or novel biomarkers for diseases. In some cases, the methods and systems may be used to facilitate identification of disease-relevant protein variants. In some cases, the methods and systems may be used to observe examples of alternative exon usage. In some cases, the methods and systems may be used to identify proteoforms arising from alternative splicing. In some cases, the methods and systems may be used to identify proteoforms arising from genetic variation. In some cases, the methods and systems may be used to identify proteoforms based at least partially on custom protein databases generated from subject-matched genotype data, such as whole exome sequencing (WES) data. In some cases, the methods and systems may be used to discover new proteoforms. In some cases, the methods and systems may be used to identify proteoforms that would otherwise not be identified using protein affinity-based targeted technologies. In some cases, the methods and systems disclosed herein may be used to support enhanced understanding of human health and disease by identifying proteoforms.

Some aspects of the methods described herein include obtaining protein information from biomolecule coronas that correspond to particles incubated with a biofluid sample (e.g., blood, serum, or plasma) from a subject; and using a classifier to identify the biofluid sample being indicative of a healthy state or a cancer state based on the protein information. Some aspects include contacting a biofluid sample from a subject suspected of having a disease state with particles such that peptides of the biofluid sample adsorb to the particles; assaying (e.g., by mass spectrometry) the peptides to obtain protein information; and identifying the subject as having the disease state or as not having the disease state based on the protein information. The protein information may include a peptide measurement. The protein information may include a protein group measurement. The protein information may include peptide measurements. The protein information may include a combination of peptide and protein group measurements. The protein information may include information on individual protein or peptide isoforms (e.g., resulting from alternative splicing). The protein information may include separate peptide measurements from a protein group, which are differentially expressed. For example, a measurement of a first peptide of a protein group may be increased (e.g., in concentration) relative to a control sample, and a measurement of a second peptide of the protein group may be decreased (e.g., in concentration) relative to a control sample, when the biofluid sample is indicative of the cancer state relative to the healthy state. The method may further include providing a cancer treatment such as surgery, chemotherapy, or radiation therapy to the subject.

Some aspects of the methods described herein include obtaining genetic information from a biofluid sample of a subject; obtaining protein information from biomolecule coronas that correspond to particles incubated with a biofluid sample from a subject; and identifying protein variants based on the genetic information. Some aspects include sequencing nucleic acids from a biofluid sample of a subject to obtain genetic information; contacting the biofluid sample with particles such that peptides of the biofluid sample adsorb to the particles; assaying the peptides to obtain protein information; and identifying protein variants from among the protein information based on the genetic information. The genetic information may include whole-exome sequencing information. The genetic information may include information on nucleotide polymorphisms that translate to amino acid polymorphisms. The protein variants may include allelic variants. Some aspects may include using a classifier to identify a biofluid sample from a subject as indicative of a healthy state or a cancer state based on a measurement of protein variants in the sample. The method may further include providing a cancer treatment such as surgery, chemotherapy, or radiation therapy to the subject based on the protein variants.

Some aspects of the methods described herein include identifying one or more genomic regions associated with a biological state based at least partially on proteomic information. The genomic regions may include one or more regions in a DNA sequence of a subject. The biological state may include a diagnosis, a prognosis, or any clinically relevant score or assessment for a subject. The proteomic information may comprise a one or more expressed proteoforms, wherein the one or more of expressed proteoforms are expressed from the one or more region in the DNA sequence.

Some aspects of the methods described herein include providing a diagnosis, a prognosis, or any clinically relevant score or assessment for a subject. In some cases, the diagnosis, the prognosis, or the clinically relevant score or assessment may be based at least partially on proteomic information and genomic information obtained from a method disclosed herein. In some cases, combining proteomic information and genomic information may provide low false positive or false negative rates for the diagnosis, the prognosis, or the clinically relevant score or assessment.

### Method for Determining Expressed Proteoforms

In some aspects, the present disclosure describes a method for assaying a biological sample. In some cases, the method comprises assaying a set of nucleic acids from the biological sample to obtain genotypic information of the biological sample. In some cases, the method comprises generating, based at least in part on the genotypic information, a set of expressible proteoforms that can be expressed from the set of nucleic acids. In some cases, the method comprises assaying a set of polyamino acids from the biological sample to generate proteomic information of the biological sample. In some cases, the method comprises mapping the set of identifications to the set of expressible proteoforms, thereby determining a set of expressed proteoforms in the biological sample. In some cases, the proteomic information comprises a set of identifications for the set of polyamino acids.

In some cases, a biological sample may comprise various biomolecules, including proteins, nucleic acids, lipids, carbohydrates, any combination thereof, and more. In some cases, the presence or absence and/or concentration of various biomolecules, as well as correlations between various subsets of biomolecules (e.g., proteins and nucleic acids), may be indicative of the biological state of a given biological sample (e.g., a healthy or a disease state). In some cases, the method may be performed with a plurality of biological samples. In some cases, a biological sample may be obtained from a subject. In some cases, a biological sample may be obtained from a plurality of subjects.

In some cases, a nucleic acid may comprise any one of various species or type of nucleic acids. In some cases, a nucleic acid may be single-stranded, double-stranded. In some cases, a nucleic acid may comprise a single-stranded portion and a double-stranded portion. In some cases, a nucleic acid may be linear, branched, or cyclic. In some cases, a nucleic acid may comprise various secondary structures, tertiary structures, or quaternary structures. In some cases, a nucleic acid may comprise a deoxyribonucleic acid (DNA) or ribonucleic acid (RNA). In some case, a nucleic acid may comprise a coding sequence, a non-coding sequence, or both. In some cases, a nucleic acid may comprise a coding or non-coding region of a gene or gene fragment, or any combination thereof. In some cases, a nucleic acid may comprise a messenger ribonucleic acid (mRNA), a DNA, a micro ribonucleic acid (miRNA), a transfer ribonucleic acid (tRNA), a long non-coding RNA (lncRNA), a ribosomal ribonucleic acid (rRNA), a small nuclear RNA (snRNA), a piwi-interacting RNA (piRNA), a small nucleolar RNA (snoRNA), an extracellular RNA (exRNA), a small cajal body-specific RNA (scaRNA), a silencing ribonucleic acid (siRNA), self-amplifying RNA (saRNA), a YRNA (small noncoding RNA), a heterogeneous nuclear RNA (HnRNA), complementary DNA (cDNA), a short-hairpin RNA (shRNA), a ribozyme, a recombinant nucleic acid, a plasmid, a vector, an isolated DNA, an isolated RNA, or any combination thereof.

In some cases, the set of polyamino acids comprises a set of proteins expressed in the biological sample. In some cases, the set of polyamino acids comprises a set of peptide fragments derived from a set of proteins expressed in the biological sample. In some cases, the set of peptide fragments is derived by trypsinization. In some cases, the set of peptide fragments comprise tryptic peptide fragments, semi-tryptic peptide fragments, or both. In some cases, the set of peptide fragments is derived by lysinization. In some cases, the proteomic information comprises a set of peptide intensities detected from assaying the set of polyamino acids. In some cases, the set of identifications comprises protein group identifications for the set of polyamino acids. In some cases, the set of identifications comprises amino acid sequences for the set of polyamino acids. In some cases, the set of identifications comprises mass spectrometry signals for the set of polyamino acids. In some cases, the set of identifications comprises mass spectrometry signals for the set of protein sequencing reads. In some cases, the set of identifications comprises post-translational modifications for the set of polyamino acids. In some cases, the mapping comprises matching an identification in the set of identifications to an expressible proteoform in the set of expressible proteoforms, thereby determining that the matched expressible proteoform is an expressed proteoform in the biological sample. In some cases, the set of expressed proteoforms comprises at least about 10, 20, 30, 40, 50, 60, 70, 80, 90, 100, 200, 300, 400, 500, 600, 700, 800, 900, or 1,000 expressed proteoforms. In some cases, the set of expressed proteoforms comprises at most about 10, 20, 30, 40, 50, 60, 70, 80, 90, 100, 200, 300, 400, 500, 600, 700, 800, 900, or 1,000 expressed proteoforms. In some cases, the set of expressed proteoforms comprises about 10-1000, 20-900, 30-800, 40-700, 50-600, 60-500, 70-400, 80-300, 90-200, or 100-150 expressed proteoforms.

In some cases, the method for assaying a biological sample comprises associating the set of expressed proteoforms with a biological state of the biological sample. In some cases, the associating is based at least partially on the expression levels of each proteoform in the set of expressed proteoforms. In some cases, the associating is based at least partially on the relative abundances of each proteoform in the set of expressed proteoforms. In some cases, the method for assaying a biological sample further comprises associating the genotypic information with the biological state of the biological sample. In some cases, the set of polyamino acids are derived from the biological sample using at least one untargeted assay. In some cases, the set of polyamino acids comprise a dynamic range of at least 5, 6, 7, 8, 9, 10, 11, or 12 orders of magnitude in the biological sample. In some cases, the proteomic information comprises a dynamic range of at least 5, 6, 7, 8, 9, 10, 11, or 12 orders of magnitude in the biological sample. In some cases, the proteomic information comprises a dynamic range of at most 5, 6, 7, 8, 9, 10, 11, 12, 13, 14, or 15 orders of magnitude in the biological sample.

In some cases, the method may further comprise at least one untargeted assay. In some cases, the at least one untargeted assay comprises providing a plurality of surface regions comprising a plurality of surface types. In some cases, the at least one untargeted assay comprises contacting the biological sample with the plurality of surface regions to yield a set of adsorbed biomolecules on the plurality of surface regions. In some cases, the at least one untargeted assay comprises desorbing, from the plurality of surface regions, at least a portion of the set of adsorbed biomolecules to yield the set of polyamino acids. In some cases, the plurality of surface regions is disposed on a single continuous surface. In some cases, the plurality of surface regions is disposed on a plurality of discrete surfaces. In some cases, the plurality of discrete surfaces is surfaces of a plurality of particles. In some cases, the at least one untargeted assay has a false discovery rate of at most about 5%, 4%, 3%, 2%, 1%, 0.9%, 0.8%, 0.7%, 0.6%, 0.5%, 0.4%, 0.3%, 0.2%, or 0.1%. In some cases, the at least one untargeted assay has a false discovery rate of about 5%-0.1%, 4%-0.2%, 3%-0.3%, 2%-0.4%, 1%-0.5%, 0.9%-0.6%, or 0.8%-0.7%. In some cases, the at least one untargeted assay has a false discovery rate of no more than about 5%, 4%, 3%, 2%, 1%, 0.9%, 0.8%, 0.7%, 0.6%, 0.5%, 0.4%, 0.3%, 0.2%, or 0.1%.

In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample.

In some cases, the plurality of surface types may comprise a surface of a particle. In some cases, the particle is a nanoparticle. In some cases, the particle is a microparticle. In some cases, the particle is a bead. In some cases, a particle may be surface functionalized.

In some aspects, the present disclosure describes a method for assaying a biological sample. In some cases, the method comprises assaying a set of peptides from the biological sample using spectral data to generate proteomic information of the biological sample. In some cases, the method comprises identifying a set of protein groups based at least in part on the spectral data of the set of peptides. In some cases, the method comprises identifying one or more sets of peptides that are correlated in abundance for a given protein group in the set of protein groups. In some cases, the method comprises mapping the set of peptides a database of human genes with isoform information, thereby determining a set of proteoforms that result in the set of peptides. In some cases, biological samples may be complex mixtures of various biomolecules, including proteins, nucleic acids, lipids, polysaccharides, and more. In some cases, the one or more samples may comprise one or more biological samples. In some cases, the one or more samples may be obtained from a subject. In some cases, the one or more samples may be obtained from a plurality of subjects. In some cases, the proteomic information comprises a set of identifications for the set of peptides.

In some cases, the spectral data comprises mass spectrometry data. In some cases, the mass spectral data are obtained from the biological sample contacting a plurality of surface types. In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample. In some cases, the plurality of surface types may comprise a surface of a particle. In some cases, the particle is a nanoparticle. In some aspects, the particle is a microparticle. In some cases, the particle is a bead. In some cases, a particle may be surface functionalized.

In some cases, the identifying the one or more sets of peptides comprises computing a correlation of abundances of each peptide across the biological sample. In some cases, the identifying the one or more sets of peptides comprises computing a correlation of abundances of each peptide across a plurality of biological samples or clustering based on peptides' correlations. In some cases, the method for assaying a biological sample further comprises, subsequent to (c), identifying a first set of peptides that are correlated in abundance; identifying a second set of peptides that are correlated in abundance; and applying a filtering step to confirm that the set of peptides are distinct from each other. In some cases, the method further comprises identifying more than two sets of peptides that are correlated in abundance, and applying a filtering step to confirm that the more than two sets of peptides are distinct from each other. In some cases, the first set of peptides comprise a first proteoform, and the second set of peptides comprise a second proteoform, wherein the first proteoform and the second proteoform are expressed from a same locus of exons. In some cases, the first set of peptides comprise a first proteoform, and the second set of peptides comprise a second proteoform, wherein the first proteoform and the second proteoform are expressed from a same locus of exons. In some cases, the biological sample comprises a plasma sample derived from a subject afflicted with a non-small cell lung cancer. In some cases, an identified proteoform is associated with a disease. In some cases, the set of proteoforms comprise peptide variants, protein variants, or both. In some cases, the set of proteoforms comprise splicing variants, allelic variants, post-translation modification variants, or any combination thereof. In some cases, the database of human genes comprises an ENSEMBL database with isoform information.

In some cases, the methods described herein include identifying proteins with distinct proteoforms. In some cases, proteoform detection in deep plasma preteomics is performed by peptide expression correlation method and genomic mapping. In some cases, the peptide abundances are calculated by the correlation method within each protein group. In some cases, the correlation method is selected from the group consisting of, but is not limited to, the Pearson pairwise correlation, the Kendall rank correlation, the Spearman correlation, the chatterjee correlation, the Point-Biserial correlation, and the like. In some cases, for the identification of clusters of similar abundant peptides, an optimal number of clusters is determined. In some cases, a silhouette method is applied to obtain an optimal number of clusters and K-means clustering on the correlation of peptide abundances is used. In some cases, the method for determining an optimal number of clusters is used in combination with clustering algorithms that requires the specification of number of clusters. In some cases, the method of determining optimal number of clusters is selected from the group consisting of, but is not limited to, Gap statistics, the Elbow Method, Calinski-Harabasz Index, Davies-Bouldin Index, the use of Dendrogram, Bayesian information criterion, and the like. In some cases, the clustering method is selected from the group consisting of, but is not limited to, any centroid-based clustering like K-means, K-medoid, k-modes, k-median, and the like. In some cases, clustering algorithm that requires no specification of number of clusters is used to cluster peptides. In some cases, the method to cluster peptides into groups for proteoform identification is selected from the group consisting of, but is not limited to, Density-based Clustering like DBSCAN and DENCAST, Distribution-based Clustering like Gaussian Mixed Models and DBCLASD, and hierarchical clustering like DIANA and AGNES.

In some cases, a filtering step is applied to ensure that the quantitative profile of peptides from different clusters are distinct. In some cases, the filtering step comprises calculating inter-cluster correlations between peptides within a cluster and peptides outside of a cluster. In some cases, the average of all inter-cluster correlations is lower than a certain threshold for the protein to be designated as a protein with distinct clusters. In some cases, the threshold is calculated based on the distribution of correlation of all proteins in the cohort, one standard deviation lower than the mean of the distribution can be used as the threshold. In some cases, peptides are mapped to protein isoforms from the ENSEMBL database as a separate process. In some cases, the presence of a proteoform is inferred if the known protein isoform explains the results of the peptide clustering.

### Method for Determining Expression Patterns of Genes

In some aspects, the present disclosure describes a method for assaying a biological sample. In some cases, the method comprises assaying a set of polyamino acids from the biological sample to generate proteomic information of the biological sample. In some cases, the proteomic information comprises a set of identifications for the set of polyamino acids. In some cases, the method comprises assaying a set of nucleic acids from the biological sample to obtain genotypic information of the biological sample. In some cases, the genotypic information comprises one or more nucleic acid sequences. In some cases, the method comprises determining an expression pattern of one or more regions in the one or more nucleic acid sequences. In some cases, the determining is based at least partially on the set of identifications.

In some cases, an expression pattern may comprise expression levels of polyamino acids associated with the one or more regions in the one or more nucleic acid sequences. In some cases, an expression pattern may comprise expression levels of polyamino acids associated with DNA associated with the one or more regions in the one or more nucleic acid sequences. In some cases, an expression pattern may comprise expression levels of polyamino acids associated with pre-mRNA associated with the one or more regions in the one or more nucleic acid sequences. In some cases, an expression pattern may comprise expression levels of polyamino acids associated with mRNA associated with the one or more regions in the one or more nucleic acid sequences. In some cases, an expression pattern may comprise expression levels of pre-mRNA associated with the one or more regions in the one or more nucleic acid sequences. In some cases, an expression pattern may comprise expression levels of mRNA associated with the one or more regions in the one or more nucleic acid sequences. In some cases, an expression pattern may comprise expression levels of at least 1, 2, 3, 4, 5, 6, 7, 8, 9, 10, 20, 30, 40, 50, 60, 70, 80, 90, or 100 polyamino acids. In some cases, an expression pattern may comprise usage patterns of one or more exons in the one or more nucleic acid sequences.

In some cases, an expression pattern may be associated with a disease state. In some cases, an expression pattern may be associated with a prognostic state. In some cases, an expression pattern may be useful as a biomarker. In some cases, an expression pattern may indicate what proteoforms may be expressed from at least a subset of the one or more nucleic acid sequences. In some cases, an expression pattern may indicate regulatory mechanisms that control transcription of at least a subset of the one or more nucleic acid sequences or translation thereof.

In some cases, the proteomic information comprises a set of identifications for the set of polyamino acids. In some cases, the genotypic information comprises one or more nucleic acid sequences. In some cases, the set of polyamino acids comprises a set of peptide fragments derived from a set of proteins expressed in the biological sample. In some cases, the set of identifications comprises protein group identifications or amino acid sequences for the set of polyamino acids. In some cases, the set of nucleic acids is an exome of the biological sample and the genotypic information comprises an exome sequence of the biological sample. In some cases, the one or more regions are one or more exons in the exome sequence. In some cases, the method may comprise determining a nucleic acid sequence with lower error rate based at least partially on the set of identifications of the polyamino acids. In some cases, the method may comprise determining an identification of a polyamino acid with lower error rate based at least partially on a nucleic acid sequence.

In some cases, the set of polyamino acids are derived from the biological sample using at least one untargeted assay. In some cases, the set of polyamino acids comprise a dynamic range of at least 5 orders of magnitude in the biological sample. In some cases, the set of polyamino acids comprise a dynamic range of at least 5, 6, 7, 8, 9, 10, 11, or 12 orders of magnitude in the biological sample. In some cases, the set of polyamino acids comprise a dynamic range of at most 5, 6, 7, 8, 9, 10, 11, or 12 orders of magnitude in the biological sample.

In some cases, the at least one untargeted assay comprises providing a plurality of surface regions comprising a plurality of surface types. In some cases, the at least one untargeted assay comprises contacting the biological sample with the plurality of surface regions to yield a set of adsorbed biomolecules on the plurality of surface regions. In some cases, the at least one untargeted assay comprises desorbing, from the plurality of surface regions, at least a portion of the set of adsorbed biomolecules to yield the set of polyamino acids. In some cases, the plurality of surface regions are disposed on a single continuous surface. In some cases, the plurality of surface regions are disposed on a plurality of discrete surfaces. In some cases, the plurality of discrete surfaces are surfaces of a plurality of particles.

In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample. In some aspects, the plurality of surface types may comprise a surface of a particle. In some aspects, the particle is a nanoparticle. In some aspects, the particle is a microparticle. In some aspects, the particle is a bead. In other cases, the particle is a synthesized particle. In some cases, a particle may be surface functionalized.

In some cases, the set of polyamino acids comprises a set of proteins expressed in the biological sample. In some cases, the set of polyamino acids comprises a set of peptide fragments derived from a set of proteins expressed in the biological sample. In some cases, the set of peptide fragments is derived by trypsinization. In some cases, the set of peptide fragments is derived by lysinization. In some cases, the proteomic information comprises a set of peptide intensities detected from assaying the set of polyamino acids. In some cases, the set of identifications comprises protein group identifications for the set of polyamino acids. In some cases, the set of identifications comprises amino acid sequences for the set of polyamino acids. In some cases, the set of identifications comprises mass spectrometry signals for the set of polyamino acids. In some cases, the set of identifications comprises post-translational modifications for the set of polyamino acids. In some cases, the mapping comprises matching an identification in the set of identifications to an expressible proteoform in the set of expressible proteoforms, thereby determining that the matched expressible proteoform is an expressed proteoform in the biological sample. In some cases, the set of expressed proteoforms comprises at least about 10, 20, 30, 40, 50, 60, 70, 80, 90, 100, 200, 300, 400, 500, 600, 700, 800, 900, or 1,000 expressed proteoforms. In some cases, the set of expressed proteoforms comprises at most about 10, 20, 30, 40, 50, 60, 70, 80, 90, 100, 200, 300, 400, 500, 600, 700, 800, 900, or 1,000 expressed proteoforms. In some cases, the set of expressed proteoforms comprises about 10-1000, 20-900, 30-800, 40-700, 50-600, 60-500, 70-400, 80-300, 90-200, or 100-150 expressed proteoforms.

In some cases, the method comprises associating the expression pattern with a biological state of the biological sample. In some cases, the associating is based at least partially on the expression levels of each proteoform in the set of expressed proteoforms. In some cases, the associating is based at least partially on the transcription levels of each nucleic acid sequence in the one or more nucleic acid sequences. In some cases, the associating is based at least partially on the relative abundances of each proteoform in the set of expressed proteoforms. In some cases, the method for assaying a biological sample further comprises associating the genotypic information with the biological state of the biological sample. In some cases, the set of polyamino acids are derived from the biological sample using at least one untargeted assay. In some cases, the set of polyamino acids comprise a dynamic range of at least 5, 6, 7, 8, 9, 10, 11, or 12 orders of magnitude in the biological sample. In some cases, the proteomic information comprises a dynamic range of at least 5, 6, 7, 8, 9, 10, 11, or 12 orders of magnitude in the biological sample. In some cases, the proteomic information comprises a dynamic range of at most 5, 6, 7, 8, 9, 10, 11, 12, 13, 14, or 15 orders of magnitude in the biological sample.

In some cases, the method may further comprise at least one untargeted assay. In some cases, the at least one untargeted assay comprises providing a plurality of surface regions comprising a plurality of surface types. In some cases, the at least one untargeted assay comprises contacting the biological sample with the plurality of surface regions to yield a set of adsorbed biomolecules on the plurality of surface regions. In some cases, the at least one untargeted assay comprises desorbing, from the plurality of surface regions, at least a portion of the set of adsorbed biomolecules to yield the set of polyamino acids. In some cases, the plurality of surface regions is disposed on a single continuous surface. In some cases, the plurality of surface regions is disposed on a plurality of discrete surfaces. In some cases, the plurality of discrete surfaces is surfaces of a plurality of particles.

In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample.

In some cases, the plurality of surface types may comprise a surface of a particle. In some cases, the particle is a nanoparticle. In some cases, the particle is a microparticle. In some cases, the particle is a bead. In some cases, a particle may be surface functionalized.

### Method for Identifying a Differentially Expressed Polyamino Acids

In some aspects, the present disclosure describes a method for identifying a differentially expressed polyamino acid. In some cases, the method comprises obtaining a plurality of polyamino acids from a plurality of biological samples. In some cases, the method comprises assaying the plurality of polyamino acids, using at least one untargeted assay, to generate a plurality of identifications for the plurality of polyamino acids. In some cases, the method comprises identifying at least one polyamino acid in the plurality of polyamino acids that is differentially expressed in the at least one clinically relevant dimension. In some cases, the plurality of biological samples are differential in at least one clinically relevant dimension. In some cases, the plurality of polyamino acids comprises one or more peptide fragments derived from proteins expressed in the plurality of biological samples. In some cases, the at least one clinically relevant dimension is a disease state. In some cases, the disease state is a presence of cancer or an absence of cancer. In some cases, the disease state is a stage of cancer. In some cases, the differentially expressed polyamino acid is upregulated when it is indicative of the disease state. In some cases, the differentially expressed polyamino acid is downregulated when it is indicative of the disease state.

In some cases, the clinically relevant dimension may be a disease state. In some cases, the clinically relevant dimension may comprise a presence or an absence of a disease. In some cases, the clinically relevant dimension may comprise severity of a disease. In some cases, the clinically relevant dimension may comprise a progression of a disease. In some cases, the clinically relevant dimension may comprise a likelihood of recovery by a patient. In some cases, the clinically relevant dimension may comprise a likelihood of success of a therapy or procedure on a patient. In some cases, the clinically relevant dimension may comprise a likelihood of a presence or an absence of a side-effect associated with a therapeutic.

In some cases, the plurality of biological samples may comprise biological samples from a population of individuals. In some cases, the population of individual may comprise a subset of individuals afflicted or suspected of being afflicted with a disease. In some cases, the population of individual may comprise a subset of healthy individuals. In some cases, the population of individuals may comprise individuals at various stages in a disease. In some cases, the population of individuals may comprise males, females, age groups, or any combination thereof. In some cases, the population of individuals may comprise individuals with various diets.

In some cases, the plurality of polyamino acids are peptide fragments derived from proteins expressed in the plurality of biological samples. In some cases, the set of polyamino acids comprise a dynamic range of at least 5 orders of magnitude in the biological sample. In some cases, the set of polyamino acids comprise a dynamic range of at least 6 orders of magnitude in the biological sample. In some cases, the set of polyamino acids comprise a dynamic range of at least 7 orders of magnitude in the biological sample. In some cases, the set of polyamino acids comprise a dynamic range of at least 8 orders of magnitude in the biological sample. In some cases, the set of polyamino acids comprise a dynamic range of at least 9 orders of magnitude in the biological sample. In some cases, the set of polyamino acids comprise a dynamic range of at least 10 orders of magnitude in the biological sample. In some cases, the set of polyamino acids comprise a dynamic range of at least 11 orders of magnitude in the biological sample. In some cases, the set of polyamino acids comprise a dynamic range of at least 12 orders of magnitude in the biological sample. In some cases, the set of polyamino acids comprise a dynamic range of at least 5, 6, 7, 8, 9, 10, 11, or 12 orders of magnitude in the biological sample.

In some cases, the at least one untargeted assay comprises providing a plurality of surface regions comprising a plurality of surface types. In some cases, the at least one untargeted assay comprises contacting the biological sample with the plurality of surface regions to yield a set of adsorbed biomolecules on the plurality of surface regions. In some cases, the at least one untargeted assay comprises desorbing, from the plurality of surface regions, at least a portion of the set of adsorbed biomolecules to yield the set of polyamino acids. In some cases, the plurality of surface regions are disposed on a single continuous surface. In some cases, the plurality of surface regions are disposed on a plurality of discrete surfaces. In some cases, the plurality of discrete surfaces are surfaces of a plurality of particles.

In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample. In some aspects, the plurality of surface types may comprise a surface of a particle. In some aspects, the particle is a nanoparticle. In some aspects, the particle is a microparticle. In some aspects, the particle is a bead. In other cases, the particle is a synthesized particle. In some cases, a particle may be surface functionalized.

In some cases, the determining comprises identifying one or more base positions in the one or more nucleic acid sequences that covaries with at least one element in the proteomic information. In some cases, the one or more base positions comprise a single nucleotide polymorphism. In some cases, the one or more base positions comprise a deletion or an insertion. In some cases, the one or more base positions comprise a methylation. In some cases, the at least one element comprises a polyamino acid identification in the set of polyamino acid identifications and a polyamino acid intensity measured using the untargeted assay. In some cases, the polyamino acid intensity is measured using mass spectrometry. In some cases, the determining further comprises filtering the one or more base positions when a statistical significance value for the one or more base pair positions is less than a threshold statistical significance value. In some cases, the statistical significance value is a p-value. In some cases, the threshold statistical significance value is equal to, greater than, or less than 1e⁻², 1e⁻³, 1e⁻⁴, 1e⁻⁵, 1e⁻⁶, 1e⁻⁷, or 1e⁻⁸.

In some cases, the determining further comprises filtering the one or more base positions when a false discovery rate for the one or more base pair positions is less than a threshold false discovery rate. In some cases, the false discovery rate is determined by: (a) shuffling the proteomic data to generate a shuffled proteomic data; (b) identifying one or more decoy base positions in a shuffled proteomic data that covaries with at least one element in the proteomic information; and (c) normalizing the number of the one or more decoy base positions by the number of the one or more base positions. In some cases, the one or more decoy base positions may be identified in multiple runs. In some cases, the number of the one or more decoy base positions may be normalized by a mean number of decoy base positions identified in multiple runs.

In some cases, the method further comprises classifying the one or more base positions as a cis-pQTL or a trans-pQTL based on a distance between the one or more base positions and a gene that encodes a polyamino acid comprising the polyamino acid identification. In some cases, the one or more base positions are classified as a cis-pQTL when the distance is less than 1 megabases (Mbp) of a transcription start site of the gene. In some cases, the one or more base positions are classified as a cis-pQTL when the distance is less than 10, 9, 8, 7, 6, 5, 4, 3, 2, 1, 0.9, 0.8, 0.7, 0.6, 0.5, 0.4, 0.3, 0.2, or 0.1 megabases (Mbp) of a transcription start site of the gene. In some cases, the distance is greater than 5 kilobases (kb) upstream. In some cases, the distance is greater than 1, 2, 3, 4, 5, 6, 7, 8, 9, 10, 20, 30, 40, or 50 kb upstream. In some cases, the distance is less than 1 kb downstream. In some cases, the distance is less than 10, 9, 8, 7, 6, 5, 4, 3, 2, 1, 0.9, 0.8, 0.7, 0.6, 0.5, 0.4, 0.3, 0.2, or 0.1 kb downstream. Otherwise, a pQTL is considered to be a trans-pQTL. In some cases, the one or more regions in the one or more nucleic acid sequences comprises the gene that encodes a polyamino acid comprising the polyamino acid identification. In some cases, a pQTL may be a biomarker for a disease.

### Method for Identifying Similarly Expressed Proteoforms in Subject Groups

In some aspects, the present disclosure describes a method for assaying a biological sample. In some cases, the method comprises assaying a set of peptides from a plurality of biological samples to obtain a set of peptide identifications. In some cases, the method comprises identifying a set of protein groups based at least in part on the set of peptide identifications. In some cases, the method comprises determining, for a given protein group in the set of protein groups, a set of correlated peptides that are correlated in abundance across the plurality of biological samples. In some cases, the method comprises mapping the set of correlated peptides to a set of expressible proteoforms. In some cases, the method comprises identifying at least one proteoform common in the plurality of biological samples.

In some cases, the plurality of biological samples may comprise biological samples from a population of individuals. In some cases, the population of individual may comprise individuals afflicted or suspected of being afflicted with a disease. In some cases, the population of individual may comprise healthy individuals. In some cases, the population of individuals may comprise individuals at a certain stage of a disease. In some cases, the population of individuals may comprise males, females, age groups, or any combination thereof. In some cases, the population of individuals may comprise individuals with a similar diet.

In some cases, the set of correlated peptides may be associated with a characteristic of the plurality of biological samples. In some cases, the set of correlated peptides may be associated with a presence or an absence of a disease. In some cases, the set of correlated peptides may be associated with a severity of a disease. In some cases, the set of correlated peptides may be associated with a stage of a disease. In some cases, the set of correlated peptides may be associated with a likelihood of recovery by a patient. In some cases, the set of correlated peptides may be associated with a likelihood of success of a therapy or procedure on a patient. In some cases, the set of correlated peptides may be associated with a likelihood of a presence or an absence of a side-effect associated with a therapeutic.

In some cases, the proteoform may be associated with a characteristic of the plurality of biological samples. In some cases, the proteoform may be associated with a presence or an absence of a disease. In some cases, the proteoform may be associated with a severity of a disease. In some cases, the proteoform may be associated with a stage of a disease. In some cases, the proteoform may be associated with a likelihood of recovery by a patient. In some cases, the proteoform may be associated with a likelihood of success of a therapy or procedure on a patient. In some cases, the proteoform may be associated with a likelihood of a presence or an absence of a side-effect associated with a therapeutic.

In some cases, the set of peptides are peptide fragments derived from proteins expressed in the plurality of biological samples. In some cases, the set of peptides comprises a dynamic range of at least 5 orders of magnitude in the biological sample. In some cases, the set of peptides comprises a dynamic range of at least 6 orders of magnitude in the biological sample. In some cases, the set of peptides comprises a dynamic range of at least 7 orders of magnitude in the biological sample. In some cases, the set of peptides comprises a dynamic range of at least 8 orders of magnitude in the biological sample. In some cases, the set of peptides comprises a dynamic range of at least 9 orders of magnitude in the biological sample. In some cases, the set of peptides comprises a dynamic range of at least 10 orders of magnitude in the biological sample. In some cases, the set of peptides comprises a dynamic range of at least 11 orders of magnitude in the biological sample. In some cases, the set of peptides comprises a dynamic range of at least 12 orders of magnitude in the biological sample. In some cases, the set of peptides comprises a dynamic range of at least 5, 6, 7, 8, 9, 10, 11, or 12 orders of magnitude in the biological sample.

In some cases, the at least one untargeted assay comprises providing a plurality of surface regions comprising a plurality of surface types. In some cases, the at least one untargeted assay comprises contacting the biological sample with the plurality of surface regions to yield a set of adsorbed biomolecules on the plurality of surface regions. In some cases, the at least one untargeted assay comprises desorbing, from the plurality of surface regions, at least a portion of the set of adsorbed biomolecules to yield the set of polyamino acids. In some cases, the plurality of surface regions are disposed on a single continuous surface. In some cases, the plurality of surface regions are disposed on a plurality of discrete surfaces. In some cases, the plurality of discrete surfaces are surfaces of a plurality of particles.

In some cases, the plurality of surface types may comprise a surface that is configured to capture or interact with a biomolecule from a sample. For example, the surface may be functionalized with nucleic acid binding moieties, such as single stranded nucleic acids, which are capable of binding nucleic acids from a sample. In some aspects, the plurality of surface types may comprise a surface of a particle. In some aspects, the particle is a nanoparticle. In some aspects, the particle is a microparticle. In some aspects, the particle is a bead. In other cases, the particle is a synthesized particle. In some cases, a particle may be surface functionalized.

### Biological Sample

The present disclosure systems and methods for assaying a biological sample. In some cases, a biological sample may comprise a cell or be cell-free. In some cases, a biological sample may comprise a biofluid, such as blood, serum, plasma, urine, or cerebrospinal fluid (CSF). In some cases, a biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract. In some cases, a biological sample may be obtained from a subject. In some cases, the subject may be a human or a non-human. In some cases, the subject may be a plant, a fungus, or an archaeon. In some cases, a biological sample can contain a plurality of proteins or proteomic data, which may be analyzed after adsorption or binding of proteins to the surfaces of the various sensor element (e.g., particle) types in a panel and subsequent digestion of protein coronas.

In some cases, a biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. In some cases, a biological sample may comprise multiple biological samples (e.g., pooled plasma from multiple subjects, or multiple tissue samples from a single subject). In some cases, a biological sample may comprise a single type of biofluid or biomaterial from a single source.

In some cases, a biological sample may be diluted or pre-treated. In some cases, a biological sample may undergo depletion (e.g., the biological sample comprises serum) prior to or following contact with a surface disclosed herein. In some cases, a biological sample may undergo physical (e.g., homogenization or sonication) or chemical treatment prior to or following contact with a surface disclosed herein. In some cases, a biological sample may be diluted prior to or following contact with a surface disclosed herein. In some cases, a dilution medium may comprise buffer or salts, or be purified water (e.g., distilled water). In some cases, a biological sample may be provided in a plurality partitions, wherein each partition may undergo different degrees of dilution. In some cases, a biological sample may comprise may undergo at least about 1.1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution.

In some cases, the biological sample may comprise a plurality of biomolecules. In some cases, a plurality of biomolecules may comprise polyamino acids. In some cases, the polyamino acids comprise peptides, proteins, or a combination thereof. In some cases, the plurality of biomolecules may comprise nucleic acids, carbohydrates, polyamino acids, or any combination thereof. A biological sample may comprise a member of any class of biomolecules, where “classes” may refer to any named category that defines a group of biomolecules having a common characteristic (e.g., proteins, nucleic acids, carbohydrates).

### Proteomic Analysis

As used herein, “proteomic analysis”, “protein analysis”, and the like, may refer to any system or method for analyzing proteins in a sample, including the systems and methods disclosed herein. The present disclosure systems and methods for assaying using one or more surface. In some cases, a surface may comprise a surface of a high surface-area material, such as nanoparticles, particles, or porous materials. As used herein, a “surface” may refer to a surface for assaying polyamino acids. When a particle composition, physical property, or use thereof is described herein, it shall be understood that a surface of the particle may comprise the same composition, the same physical property, or the same use thereof, in some cases. Similarly, when a surface composition, physical property, or use thereof is described herein, it shall be understood that a particle may comprise the surface to comprise the same composition, the same physical property, or the same use thereof.

Materials for particles and surfaces may include metals, polymers, magnetic materials, and lipids. In some cases, magnetic particles may be iron oxide particles. Examples of metallic materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron, cadmium, or any alloys thereof. In some cases, a particle disclosed herein may be a magnetic particle, such as a superparamagnetic iron oxide nanoparticle (SPION). In some cases, a magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material).

The present disclosure describes panels of particles or surfaces. In some cases, a panel may comprise more than one distinct surface types. Panels described herein can vary in the number of surface types and the diversity of surface types in a single panel. For example, surfaces in a panel may vary based on size, polydispersity, shape and morphology, surface charge, surface chemistry and functionalization, and base material. In some cases, panels may be incubated with a sample to be analyzed for polyamino acids, polyamino acid concentrations, nucleic acids, nucleic acid concentrations, or any combination thereof. In some cases, polyamino acids in the sample adsorb to distinct surfaces to form one or more adsorption layers of biomolecules. The identity of the biomolecules and concentrations thereof in the one or more adsorption layers may depend on the physical properties of the distinct surfaces and the physical properties of the biomolecules. Thus, each surface type in a panel may have differently adsorbed biomolecules due to adsorbing a different set of biomolecules, different concentrations of a particular biomolecules, or a combination thereof. Each surface type in a panel may have mutually exclusive adsorbed biomolecules or may have overlapping adsorbed biomolecules.

In some cases, panels disclosed herein can be used to identify the number of distinct biomolecules disclosed herein over a wide dynamic range in a given biological sample. For example, a panel may enrich a subset of biomolecules in a sample, which can be identified over a wide dynamic range at which the biomolecules are present in a sample (e.g., a plasma sample). In some cases, the enriching may be selective—e.g., biomolecules in the subset may be enriched but biomolecules outside of the subset may not enriched and/or be depleted. In some cases, the subset may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the panel including any number of distinct particle types disclosed herein, enriches and identifies a single protein or protein group by binding different domains, sequences, or epitopes of the protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group. In some cases, a panel including any number of distinct particle types disclosed herein, may enrich and identify biomolecules over a dynamic range of at least 5, 6, 7, 8, 9, 10, 15, or 20 magnitudes. In some cases, a panel including any number of distinct particle types disclosed herein, may enrich and identify biomolecules over a dynamic range of at most 5, 6, 7, 8, 9, 10, 15, or 20 magnitudes.

A panel can have more than one surface type. Increasing the number of surface types in a panel can be a method for increasing the number of proteins that can be identified in a given sample.

A particle or surface may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated or may comprise a degree of phase separation.

Examples of lipids that can be used to form the particles or surfaces of the present disclosure include cationic, anionic, and neutrally charged lipids. For example, particles and/or surfaces can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, cholesterol, and any combination thereof.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl)diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

Distinct surfaces or distinct particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group. A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

A particle may comprise a single surface such as a specific small molecule, or a plurality of surface functionalizations, such as a plurality of different small molecules. Surface functionalization can influence the composition of a particle's biomolecule corona. Such surface functionalization can include small molecule functionalization or macromolecular functionalization. A surface functionalization may be coupled to a particle material such as a polymer, metal, metal oxide, inorganic oxide (e.g., silicon dioxide), or another surface functionalization.

A surface functionalization may comprise a binding molecule. The binding molecule may be a small molecule, an oligomer, or a macromolecule. The binding molecule may comprise an binding specificity for a group or class of analytes (e.g., a plurality of saccharides or a class of proteins). A binding molecule may comprise a moderate binding specificity for the group or class of analytes. Conversely, a binding molecule may comprise a dis-affinity for a group or class of analytes, disfavoring binding of these species relative to the same particle lacking the binding molecule. For example, a binding molecule may comprise a negative charge distribution which repels negatively charged nucleic acids, thereby disfavoring their binding.

A binding molecule may comprise a peptide. Peptides are an extensive and diverse set of biomolecules which may comprise a wide range of physical and chemical properties. Depending on its composition, sequence, and chemical modification, a peptide may be hydrophilic, hydrophobic, amphiphilic, lipophilic, lipophobic, positively charged, negatively charged, zwitterionic, neutral, chaotropic, antichaotropic, reactive, redox active, inert, acidic, basic, rigid, flexible, or any combination thereof. Accordingly, a peptide surface functionalization may confer a range of physicochemical properties to a particle. A particle may comprise a single peptide surface functionalization or a plurality of peptide surface functionalizations. A single peptide surface functionalization may comprise a plurality of identical or sequence-sharing peptides bound to a particle in a uniform fashion.

A surface functionalization may comprise a small molecule functionalization, a macromolecular functionalization, or a combination of two or more such functionalizations. In some cases, a macromolecular functionalization may comprise a biomacromolecule, such as a protein or a polynucleotide (e.g., a 100-mer DNA molecule). A macromolecular functionalization may comprise a protein, polynucleotide, or polysaccharide, or may be comparable in size to any of the aforementioned classes of species. In some cases, a surface functionalization may comprise an ionizable moiety. In some cases, a surface functionalization may comprise pKa of at least about 1, 2, 3, 4, 5, 6, 7, 8, 9, 10, 11, 12, 13, or 14. In some cases, a surface functionalization may comprise pKa of at most about 1, 2, 3, 4, 5, 6, 7, 8, 9, 10, 11, 12, 13, or 14. In some cases, a small molecule functionalization may comprise a small organic molecule such as an alcohol (e.g., octanol), an amine, an alkane, an alkene, an alkyne, a heterocycle (e.g., a piperidinyl group), a heteroaromatic group, a thiol, a carboxylate, a carbonyl, an amide, an ester, a thioester, a carbonate, a thiocarbonate, a carbamate, a thiocarbamate, a urea, a thiourea, a halogen, a sulfate, a phosphate, a monosaccharide, a disaccharide, a lipid, or any combination thereof. For example, a small molecule functionalization may comprise a phosphate sugar, a sugar acid, or a sulfurylated sugar.

In some cases, a macromolecular functionalization may comprise a specific form of attachment to a particle. In some cases, a macromolecule may be tethered to a particle via a linker. In some cases, the linker may hold the macromolecule close to the particle, thereby restricting its motion and reorientation relative to the particle, or may extend the macromolecule away from the particle. In some cases, the linker may be rigid (e.g., a polyolefin linker) or flexible (e.g., a nucleic acid linker). In some cases, a linker may be at least about 0.5, 1, 2, 3, 4, 5, 6, 7, 8, 9, 10, 15, 20, 25, or 30 nm in length. In some cases, a linker may be at most about 0.5, 1, 2, 3, 4, 5, 6, 7, 8, 9, 10, 15, 20, 25, or 30 nm in length. As such, a surface functionalization on a particle may project beyond a primary corona associated with the particle. In some cases, a surface functionalization may also be situated beneath or within a biomolecule corona that forms on the particle surface. In some cases, a macromolecule may be tethered at a specific location, such as at a protein's C-terminus, or may be tethered at a number of possible sites. For example, a peptide may be covalent attached to a particle via any of its surface exposed lysine residues.

In some embodiments, a macromolecule can be modified with a peptide. In some embodiments, the macromolecule comprises a thiol or azide. In some embodiments, a surface comprises the macromolecule modified with a peptide immobilized to a surface. In some embodiments, the macromolecule is covalently coupled to the surface. In some embodiments, the macromolecule is electrostatically coupled to the surface. In some embodiments, the macromolecule is coupled to the surface through a polymerization event. In some embodiments, the polymerization event comprises a reaction with a vinyl group on the surface.

In some embodiments, macromolecules modified with peptides can be immobilized on surfaces for identification, binding, or enrichment of biomolecules (e.g., proteins). In some embodiments, a surface can comprise a macromolecule modified with a peptide, wherein the peptide comprises a binding site, and a protein interacting with the peptide at the binding site. In some embodiments, a biological sample can be contacted with a surface comprising the macromolecule modified with a peptide, wherein the peptides are configured to bind to a protein, which can release the plurality of biomolecules from the surface.

In some cases, a particle may be contacted with a biological sample (e.g., a biofluid) to form a biomolecule corona. In some cases, a biomolecule corona may comprise at least two biomolecules that do not share a common binding motif. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry or protein sequencing. In some cases, a single particle type may be contacted with a biological sample. In some cases, a plurality of particle types may be contacted to a biological sample. In some cases, the plurality of particle types may be combined and contacted to the biological sample in a single sample volume. In some cases, the plurality of particle types may be sequentially contacted to a biological sample and separated from the biological sample prior to contacting a subsequent particle type to the biological sample. In some cases, adsorbed biomolecules on the particle may have compressed (e.g., smaller) dynamic range compared to a given original biological sample.

In some cases, the particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

In some cases, a method of the present disclosure may identify a large number of unique biomolecules (e.g., proteins) in a biological sample (e.g., a biofluid). In some cases, a surface disclosed herein may be incubated with a biological sample to adsorb at least 1000, 2000, 3000, 4000, 5000, 6000, 7000, 8000, 9000, or 10000 unique biomolecules. In some cases, a surface disclosed herein may be incubated with a biological sample to adsorb at most 1000, 2000, 3000, 4000, 5000, 6000, 7000, 8000, 9000, or 10000 unique biomolecules. In some cases, a surface disclosed herein may be incubated with a biological sample to adsorb at least 1000, 2000, 3000, 4000, 5000, 6000, 7000, 8000, 9000, or 10000 unique biomolecule groups. In some cases, a surface disclosed herein may be incubated with a biological sample to adsorb at most 1000, 2000, 3000, 4000, 5000, 6000, 7000, 8000, 9000, or 10000 unique biomolecule groups. In some cases, several different types of surfaces can be used, separately or in combination, to identify large numbers of proteins in a particular biological sample. In other words, surfaces can be multiplexed in order to bind and identify large numbers of biomolecules in a biological sample.

In some cases, a method of the present disclosure may identify a large number of unique proteoforms in a biological sample. In some cases, a method may identify at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a method may identify at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, a surface disclosed herein may be incubated with a biological sample to adsorb at most 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 unique proteoforms. In some cases, several different types of surfaces can be used, separately or in combination, to identify large numbers of proteins in a particular biological sample. In other words, surfaces can be multiplexed in order to bind and identify large numbers of biomolecules in a biological sample.

Biomolecules collected on particles may be subjected to further analysis. In some cases, a method may comprise collecting a biomolecule corona or a subset of biomolecules from a biomolecule corona. In some cases, the collected biomolecule corona or the collected subset of biomolecules from the biomolecule corona may be subjected to further particle-based analysis (e.g., particle adsorption). In some cases, the collected biomolecule corona or the collected subset of biomolecules from the biomolecule corona may be purified or fractionated (e.g., by a chromatographic method). In some cases, the collected biomolecule corona or the collected subset of biomolecules from the biomolecule corona may be analyzed (e.g., by mass spectrometry or protein sequencing).

In some cases, the panels disclosed herein can be used to identify a number of proteins, peptides, protein groups, or protein classes using a protein analysis workflow described herein (e.g., a protein corona analysis workflow). In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 unique proteins. In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, or 100000 protein groups. In some cases, the panels disclosed herein can be used to identify at least 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, the panels disclosed herein can be used to identify at most 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, or 1000000 peptides. In some cases, a peptide may be a tryptic peptide. In some cases, a peptide may be a semi-tryptic peptide. In some cases, protein analysis may comprise contacting a sample to distinct surface types (e.g., a particle panel), forming adsorbed biomolecule layers on the distinct surface types, and identifying the biomolecules in the adsorbed biomolecule layers (e.g., by mass spectrometry or protein sequencing). Feature intensities, as disclosed herein, may refer to the intensity of a discrete spike (“feature”) seen on a plot of mass to charge ratio versus intensity from a mass spectrometry run of a sample. In some cases, these features can correspond to variably ionized fragments of peptides and/or proteins. In some cases, using the data analysis methods described herein, feature intensities can be sorted into protein groups. In some cases, protein groups may refer to two or more proteins that are identified by a shared peptide sequence. In some cases, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). In some cases, if the peptide sequence is unique to a single protein (Protein 1), a protein group could be the “ZZX” protein group having one member (Protein 1). In some cases, each protein group can be supported by more than one peptide sequence. In some cases, protein detected or identified according to the instant disclosure can refer to a distinct protein detected in the sample (e.g., distinct relative to other proteins detected using mass spectrometry). In some cases, analysis of proteins present in distinct coronas corresponding to the distinct surface types in a panel yields a high number of feature intensities. In some cases, this number decreases as feature intensities are processed into distinct peptides, further decreases as distinct peptides are processed into distinct proteins, and further decreases as peptides are grouped into protein groups (two or more proteins that share a distinct peptide sequence).

In some cases, the methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. In some cases, the methods disclosed herein provide for isolating or separating a particle type from unbound protein in a sample. In some cases, a particle type may be separated by a variety of means, including but not limited to magnetic separation, centrifugation, filtration, or gravitational separation. In some cases, particle panels may be incubated with a plurality of spatially isolated samples, wherein each spatially isolated sample is in a well in a well plate (e.g., a 96-well plate). In some cases, the particle in each of the wells of the well plate can be separated from unbound protein present in the spatially isolated samples by placing the entire plate on a magnet. In some cases, this simultaneously pulls down the superparamagnetic particles in the particle panel. In some cases, the supernatant in each sample can be removed to remove the unbound protein. In some cases, these steps (incubate, pull down) can be repeated to effectively wash the particles, thus removing residual background unbound protein that may be present in a sample.

In some cases, the systems and methods disclosed herein may also elucidate protein classes or interactions of the protein classes. In some cases, a protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); proteins that are functionally related (e.g., part of a same metabolic pathway); or proteins bearing a post-translational modification (e.g., ubiquitinated or citrullinated proteins). In some cases, a protein class may contain at least 2 proteins, 5 proteins, 10 proteins, 20 proteins, 40 proteins, 60 proteins, 80 proteins, 100 proteins, 150 proteins, 200 proteins, or more.

In some cases, the proteomic data of the biological sample can be identified, measured, and quantified using a number of different analytical techniques. For example, proteomic data can be generated using SDS-PAGE or any gel-based separation technique. In some cases, peptides and proteins can also be identified, measured, and quantified using an immunoassay, such as ELISA. In some cases, proteomic data can be identified, measured, and quantified using mass spectrometry, high performance liquid chromatography, LC-MS/MS, Edman Degradation, immunoaffinity techniques, protein sequencing, and other protein separation techniques.

In some cases, an assay may comprise protein collection of particles, protein digestion, and mass spectrometric analysis (e.g., MS, LC-MS, LC-MS/MS). In some cases, the digestion may comprise chemical digestion, such as by cyanogen bromide or 2-Nitro-5-thiocyanatobenzoic acid (NTCB). In some cases, the digestion may comprise enzymatic digestion, such as by trypsin or pepsin. In some cases, the digestion may comprise enzymatic digestion by a plurality of proteases. In some cases, the digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. In some cases, the digestion may cleave peptides at random positions. In some cases, the digestion may cleave peptides at a specific position (e.g., at methionines) or sequence (e.g., glutamate-histidine-glutamate). In some cases, the digestion may enable similar proteins to be distinguished. For example, an assay may resolve 8 distinct proteins as a single protein group with a first digestion method, and as 8 separate proteins with distinct signals with a second digestion method. In some cases, the digestion may generate an average peptide fragment length of 8 to 15 amino acids. In some cases, the digestion may generate an average peptide fragment length of 12 to 18 amino acids. In some cases, the digestion may generate an average peptide fragment length of 15 to 25 amino acids. In some cases, the digestion may generate an average peptide fragment length of 20 to 30 amino acids. In some cases, the digestion may generate an average peptide fragment length of 30 to 50 amino acids.

In some cases, an assay may rapidly generate and analyze proteomic data. In some cases, beginning with an input biological sample (e.g., a buccal or nasal smear, plasma, or tissue), a method of the present disclosure may generate and analyze proteomic data in less than about 1, 2, 3, 4, 5, 6, 7, 8, 12, 16, 20, 24, or 48 hours. In some cases, the analyzing may comprise identifying a protein group. In some cases, the analyzing may comprise identifying a protein class. In some cases, the analyzing may comprise quantifying an abundance of a biomolecule, a peptide, a protein, protein group, or a protein class. In some cases, the analyzing may comprise identifying a ratio of abundances of two biomolecules, peptides, proteins, protein groups, or protein classes. In some cases, the analyzing may comprise identifying a biological state.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo (ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 μm in diameter, a silica amino functionalized microparticle of about 0.1-0.39 μm in diameter, a Jeffamine surface particle of about 0.1-0.39 μm in diameter, a polystyrene microparticle of about 2.0-2.9 μm in diameter, a silica particle, a carboxylated particle with an original coating of about 50 nm in diameter, a particle coated with a dextran based coating of about 0.13 μm in diameter, or a silica silanol coated particle with low acidity. In some cases, a particle may lack functionalized specific binding moieties for specific binding on its surface. In some cases, a particle may lack functionalized proteins for specific binding on its surface. In some cases, a surface functionalized particle does not comprise an antibody or a T cell receptor, a chimeric antigen receptor, a receptor protein, or a variant or fragment thereof. In some cases, the ratio between surface area and mass can be a determinant of a particle's properties. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have significantly higher surface area to mass ratios, stemming in part from the higher order dependence on diameter by mass than by surface area. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg. A particle may comprise a wide array of physical properties. A physical property of a particle may include composition, size, surface charge, hydrophobicity, hydrophilicity, amphipathicity, surface functionality, surface topography, surface curvature, porosity, core material, shell material, shape, zeta potential, and any combination thereof. A particle may have a core-shell structure. In some cases, a core material may comprise metals, polymers, magnetic materials, paramagnetic materials, oxides, and/or lipids. In some cases, a shell material may comprise metals, polymers, magnetic materials, oxides, and/or lipids.

In some embodiments, proteomic information can be obtained using protein sequencing. Protein sequencing can comprise digesting a plurality of proteins to generate a plurality of protein fragments. The protein sequencing can comprise immobilizing the plurality of protein fragments to a semiconductor substrate. The protein sequencing can comprise contacting the plurality of protein fragments with a plurality of labeled recognizers. The plurality of labeled recognizers can be configured to attach to a predetermined chemical moiety in the plurality of protein fragments at the N-terminus of the plurality of protein fragments. The protein sequencing can comprise exciting the plurality of labeled recognizers to detect the plurality of labeled recognizers, thereby detecting the predetermined chemical moiety. The protein sequencing can comprise removing an amino acid from the N-terminus of the plurality of protein fragments. The protein sequencing can comprise contacting the plurality of protein fragments with a second plurality of labeled recognizers. The protein sequencing can comprise exciting the second plurality of labeled recognizers to detect a second amino acid from the N-terminus of the plurality of protein fragments, thereby performing the protein sequencing.

### Proteomic Information

In some cases, proteomic information or data can refer to information about substances comprising a peptide and/or a protein component. In some cases, proteomic information may comprise primary structure information, secondary structure information, tertiary structure information, or quaternary information about the peptide or a protein. In some cases, proteomic information may comprise information about protein-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof.

In some cases, proteomic information may comprise information about a single cell, a tissue, an organ, a system of tissues and/or organs (such as cardiovascular, respiratory, digestive, or nervous systems), or an entire multicellular organism. In some cases, proteomic information may comprise information about an individual (e.g., an individual human being or an individual bacterium), or a population of individuals (e.g., human beings with diagnosed with cancer or a colony of bacteria). Proteomic information may comprise information from various forms of life, including forms of life from the Archaea, the Bacteria, the Eukarya, the Protozoa, the Chromista, the Plantae, the Fungi, or from the Animalia. In some cases, proteomic information may comprise information from viruses.

In some cases, proteomic information may comprise information relating exons and introns in the code of life. In some cases, proteomic information may comprise information regarding variations in the primary structure, variations in the secondary structure, variations in the tertiary structure, or variations in the quaternary structure of peptides and/or proteins. In some cases, proteomic information may comprise information regarding variations in the expression of exons, including alternative splicing variations, structural variations, or both. In some cases, proteomic information may comprise conformation information, post-translational modification information, chemical modification information (e.g., phosphorylation), cofactor (e.g., salts or other regulatory chemicals) association information, or substrate association information of peptides and/or proteins.

In some cases, proteomic information may comprise information related to various proteoforms in a sample. In some cases, a proteomic information may comprise information related to peptide variants, protein variants, or both. In some cases, a proteomic information may comprise information related to splicing variants, allelic variants, post-translation modification variants, or any combination thereof.

In some cases, splicing variant (in some cases also referred to as “alternative splicing” variants, “differential splicing” variants, or “alternative RNA splicing” variants) may refer to a protein that is expressed by an alternative splicing process. In some cases, an alternative splicing process may express one or more splicing variants from a set of exons via different combinations of exons. In some cases, a combination may comprise a different sequence of exons compared to another combination. In some cases, a combination may comprise a different subset of exons compared to another combination. In some cases, a splicing variant may comprise a reordered amino acid sequence of another splicing variant.

In some cases, an allelic variant may refer to a protein that is expressed from a gene comprising a mutation compared to a reference gene. In some cases, the reference gene may be the gene of a cell, an individual, or a population of individuals. In some cases, the mutation may be a base substitution, a base deletion, or a base insertion of a genetic sequence of the gene compared to a genetic reference of the reference gene. In some cases, an allelic variant may comprise an amino acid substitution in an amino acid sequence of another allelic variant.

In some cases, a post-translation modification may refer to a protein that is modified after expression. A protein may be modified by various enzymes. In some cases, an enzyme that can modify a protein may be a kinase, a protease, a ligase, a phosphatase, a transferase, a phosphotransferase, or any other enzyme for performing the any one of modifications disclosed herein.

In some cases, peptide variants or protein variants may comprise a post-translation modification. In some cases, the post-translational modification comprises acylation, alkylation, prenylation, flavination, amination, deamination, carboxylation, decarboxylation, nitrosylation, halogenation, sulfurylation, glutathionylation, oxidation, oxygenation, reduction, ubiquitination, SUMOylation, neddylation, myristoylation, palmitoylation, isoprenylation, farnesylation, geranylgeranylation, glypiation, glycosylphosphatidylinositol anchor formation, lipoylation, heme functionalization, phosphorylation, phosphopantetheinylation, retinylidene Schiff base formation, diphthamide formation, ethanolamine phosphoglycerol functionalization, hypusine formation, beta-Lysine addition, acetylation, formylation, methylation, amidation, amide bond formation, butyrylation, gamma-carboxylation, glycosylation, polysialylation, malonylation, hydroxylation, iodination, nucleotide addition, phosphate ester formation, phosphoramidate formation, adenylation, uridylylation, propionylation, pyroglutamate formation, gluthathionylation, sulfenylation, sulfinylation, sulfonylation, succinylation, sulfation, glycation, carbonylation, isopeptide bond formation, biotinylation, carbamylation, oxidation, pegylation, citrullination, deamidation, eliminylation, disulfide bond formation, proteolytic cleavage, isoaspartate formation, racemization, protein splicing, chaperon-assisted folding, or any combination thereof.

In some cases, proteomic information may be encoded as digital information. In some cases, the proteomic information may comprise one or more elements that represents the proteomic information. In some cases, an element may represent a primary structure information, secondary structure information, tertiary structure information, or quaternary information about a peptide or a protein. In some cases, an element may represent protein-ligand interactions for a peptide or a protein. In some cases, an element may represent a source of a peptide or protein (e.g., a specific cell, tissue, organ, organism, individual, or population of inidividuals). In some cases, an element may represent a type of proteoform. In some cases, an element may be a number, a vector, an array, or any other datatypes provided herein.

### Genomic Analysis

As used herein, “genomic analysis”, “nucleic acid analysis”, and the like, may refer to any system or method for analyzing proteins in a sample, including the systems and methods disclosed herein. The present disclosure describes various compositions and methods for analyzing (e.g., detecting or sequencing) nucleic acids. In some cases, genotypic (or genomic) information may be obtained using some of the compositions and methods of the present disclosure. In some cases, genotypic information can refer to information about substances comprising a nucleotide and/or a nucleic acid component. In some cases, genotypic information may comprise epigenetic information. In some cases, epigenetic information may comprise histone modification, DNA methylation, accessibility of different regions in a genome, dynamics changes thereof, or any combination thereof. In some cases, genotypic information may comprise primary structure information, secondary structure information, tertiary structure information, or quaternary information about a nucleic acid. In some cases, genotypic information may comprise information about nucleic acid-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof. In some cases, genotypic information may comprise a rate or prevalence of apoptosis of a healthy cell or a diseased cell. In some cases, genotypic information may comprise a state of a cell, such as a healthy state or a diseased state. In some cases, genotypic information may comprise chemical modification information of a nucleic acid molecule. In some cases, a chemical modification may comprise methylation, demethylation, amination, deamination, acetylation, oxidation, oxygenation, reduction, or any combination thereof. In some cases, genotypic information may comprise information regarding from which type of cell a biological sample originates. In some cases, genotypic information may comprise information about an untranslated region of nucleic acids.

In some cases, genotypic information may comprise information about a single cell, a tissue, an organ, a system of tissues and/or organs (such as cardiovascular, respiratory, digestive, or nervous systems), or an entire multicellular organism. In some cases, genotypic information may comprise information about an individual (e.g., an individual human being or an individual bacterium), or a population of individuals (e.g., human beings with diagnosed with cancer or a colony of bacteria). Genotypic information may comprise information from various forms of life, including forms of life from the Archaea, the Bacteria, the Eukarya, the Protozoa, the Chromista, the Plantae, the Fungi, or from the Animalia. In some cases, genotypic information may comprise information from viruses.

In some cases, genotypic information may comprise information relating to exons and introns in the code of life. In some cases, genotypic information may comprise information regarding variations or mutations in the primary structure of nucleic acids, including base substitutions, deletions, insertions, or any combination thereof. In some cases, genotypic information may comprise information regarding the inclusion of non-canonical nucleobases in nucleic acids. In some cases, genotypic information may comprise information regarding variations or mutations in epigenetics.

In some cases, genotypic information may comprise information regarding variations in the primary structure, variations in the secondary structure, variations in the tertiary structure, or variations in the quaternary structure of peptides and/or proteins that one or more nucleic acids encode.

In some cases, the set of nucleic acids comprise an exome of the biological sample. In some cases, the set of nucleic acids comprise a genome, an epigenome, a transcriptome, a metabolome, a secretome, or any combination thereof. In some cases, the set of nucleic acids comprises a portion of the exome of the biological sample. In some cases, the set of nucleic acids comprise a portion of the genome, an epigenome, a transcriptome, a metabolome, a secretome, or any combination thereof. In some cases, the genotypic information comprises an exome sequence of the biological sample. In some cases, the genotypic information comprises one or more sequences of the genome, an epigenome, a transcriptome, a metabolome, a secretome, or any combination thereof.

Various sequencing methods and various sequencing reagents may be used to obtain genotypic information. In some cases, the sequencing methods disclosed herein may comprise enriching one or more nucleic acid molecules from a sample. This may comprise enrichment in solution, enrichment on a sensor element (e.g., a particle), enrichment on a substrate (e.g., a surface of an Eppendorf tube), or selective removal of a nucleic acid (e.g., by sequence-specific affinity precipitation). Enrichment may comprise amplification, including differential amplification of two or more different target nucleic acids. Differential amplification may be based on sequence, CG-content, or post-transcriptional modifications, such as methylation state. In some cases, enrichment may comprise hybridization methods, such as pull-down methods. For example, a substrate partition may comprise immobilized nucleic acids capable of hybridizing to nucleic acids of a particular sequence, and thereby capable of isolating particular nucleic acids from a complex biological solution. In some cases, hybridization may target genes, exons, introns, regulatory regions, splice sites, reassembly genes, among other nucleic acid targets. In some cases, hybridization can utilize a pool of nucleic acid probes that are designed to target multiple distinct sequences, or to tile a single sequence.

Enrichment may comprise a hybridization reaction and may generate a subset of nucleic acid molecules from a biological sample. Hybridization may be performed in solution, on a substrate surface (e.g., a wall of a well in a microwell plate), on a sensor element, or any combination thereof. A hybridization method may be sensitive for single nucleotide polymorphisms. For example, a hybridization method may comprise molecular inversion probes.

Enrichment may also comprise amplification. Suitable amplification methods include polymerase chain reaction (PCR), solid-phase PCR, RT-PCR, qPCR, multiplex PCR, touchdown PCR, nanoPCR, nested PCR, hot start PCR, helicase-dependent amplification, loop mediated isothermal amplification (LAMP), self-sustained sequence replication, nucleic acid sequence based amplification, strand displacement amplification, rolling circle amplification, ligase chain reaction, and any other suitable amplification technique.

The sequencing may target a specific sequence or region of a genome. The sequencing may target a type of sequence, such as exons. In some cases, the sequencing comprises exome sequencing. In some cases, the sequencing comprises whole exome sequencing. The sequencing may target chromatinated or non-chromatinated nucleic acids. The sequencing may be sequence-non specific (e.g., provide a reading regardless of the target sequence). The sequencing may target a polymerase accessible region of the genome. The sequencing may target nucleic acids localized in a part of a cell, such as the mitochondria or the cytoplasm. The sequencing may target nucleic acids localized in a cell, tissue, or an organ. The sequencing may target RNA, DNA, any other nucleic acid, or any combination thereof.

‘Nucleic acid’ may refer to a polymeric form of nucleotides of any length, in single-, double- or multi-stranded form. A nucleic acid may comprise any combination of ribonucleotides, deoxyribonucleotides, and natural and non-natural analogues thereof, including 5-bromouracil, peptide nucleic acids, locked nucleotides, glycol nucleotides, threose nucleotides, dideoxynucleotides, 3′-deoxyribonucleotides, dideoxyribonucleotides, 7-deaza-GTP, fluorophores-bound nucleotides, thiol containing nucleotides, biotin linked nucleotides, methyl-7-guanosine, methylated nucleotides, inosine, thiouridine, pseudourdine, dihydrouridine, queuosine, and wyosine. A nucleic acid may comprise a gene, a portion of a gene, an exon, an intron, messenger RNA (mRNA), transfer RNA (tRNA), ribosomal RNA (rRNA), short interfering RNA (siRNA), short-hairpin RNA (shRNA), micro-RNA (miRNA), a ribozyme, cDNA, a recombinant nucleic acid, a branched nucleic acid, a plasmid, cell-free DNA (cfDNA), cell-free RNA (cfRNA), genomic DNA, mitochondrial DNA (mtDNA), circulating tumor DNA (ctDNA), long non-coding RNA, telomerase RNA, Piwi-interacting RNA, small nuclear RNA (snRNA), small interfering RNA, YRNA, circular RNA, small nucleolar RNA, or pseudogene RNA. A nucleic acid may comprise a DNA or RNA molecule. A nucleic acid may also have a defined 3-dimensional structure. In some cases, a nucleic acid may comprise a non-canonical nucleobase or a nucleotide, such as hypoxanthine, xanthine, 7-methylguanine, 5,6-dihydrouracil, 5-methylcytosine, or any combination thereof. Nucleic acids may also comprise non-nucleic acid molecules.

A nucleic acid may be derived from various sources. In some cases, a nucleic acid may be derived from an exosome, an apoptotic body, a tumor cell, a healthy cell, a virtosome, an extracellular membrane vesicle, a neutrophil extracellular trap (NET), or any combination thereof.

A nucleic acid may comprise various lengths. In some cases, a nucleic acid may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 nucleotides. In some cases, a nucleic acid may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 nucleotides.

Various reagents may be used for sequencing. In some cases, a reagent may comprise primers, oligonucleotides, switch oligonucleotides, adapters, amplification adapters, polymerases, dNTPs, co-factors, buffers, enzymes, ionic co-factors, ligase, reverse transcriptase, restriction enzymes, endonucleases, transposase, protease, proteinase K, DNase, RNase, lysis agents, lysozymes, achromopeptidase, lysostaphin, labiase, kitalase, lyticase, inhibitors, inactivating agents, chelating agents, EDTA, crowding agents, reducing agents, DTT, surfactants, TritonX-IOO, Tween 20, sodium dodecyl sulfate, sarcosyl, or any combination thereof.

Various methods for sequencing nucleic acids may be used. In some cases, sequencing may comprise sequencing a whole genome or portions thereof. Sequencing may comprise sequencing a whole genome, a whole exome, portions thereof (e.g., a panel of genes, including potentially coding and non-coding regions thereof). Sequencing may comprise sequencing a transcriptome or portion thereof. Sequencing may comprise sequencing an exome or portion thereof. Sequencing coverage may be optimized based on analytical or experimental setup, or desired sequencing footprint. In some cases, a nucleic acid sequencing method may comprise high-throughput sequencing, next-generation sequencing, flow sequencing, massively-parallel sequencing, shotgun sequencing, single-molecule real-time sequencing, ion semiconductor sequencing, electrophoretic sequencing, pyrosequencing, sequencing by synthesis, combinatorial probe anchor synthesis sequencing, sequencing by ligation, nanopore sequencing, GenapSys sequencing, chain termination sequencing, polony sequencing, 454 pyrosequencing, reversible terminated chemistry sequencing, heliscope single molecule sequencing, tunneling currents DNA sequencing, sequencing by hybridization, clonal single molecule array sequencing, sequencing with MS, DNA-seq, RNA-seq, ATAC-seq, methyl-seq, ChIP-seq, or any combination thereof. The sequencing methods of the present disclosure may involve sequence analysis of RNA. RNA sequences or expression levels may be analyzed by using a reverse transcription reaction to generate complementary DNA (cDNA) molecules from RNA for sequencing or by using reverse transcription polymerase chain reaction for quantification of expression levels. The sequencing methods of the present disclosure may detect RNA structural variants and isoforms, such as splicing variants and structural variants. The sequencing methods of the present disclosure may quantify RNA sequences or structural variants. In some cases, a sequencing may method comprise spatial sequencing, single-cell sequencing or any combination thereof.

In some cases, nucleic acids may be processed by standard molecular biology techniques for downstream applications. In some cases, nucleic acids may be prepared from nucleic acids isolated from a sample of the present disclosure. In some cases, the nucleic acids may subsequently be attached to an adaptor polynucleotide sequence, which may comprise a double stranded nucleic acid. In some cases, the nucleic acids may be end repaired prior to attaching to the adaptor polynucleotide sequences. In some cases, adaptor polynucleotides may be attached to one or both ends of the nucleotide sequences. In some cases, the same or different adaptor may be bound to each end of the fragment, thereby producing an “adaptor-nucleic acid-adaptor” construct. In some cases, a plurality of the same or different adaptor may be bound to each end of the fragment. In some cases, different adaptors may be attached to each end of the nucleic acid when adaptors are attached to both ends of the nucleic acid.

In some cases, an oligonucleotide tag complementary to a sequencing primer may be incorporated with adaptors attached to a target nucleic acid. For analysis of multiple samples, different oligonucleotide tags complementary to separate sequencing primers may be incorporated with adaptors attached to a target nucleic acid.

In some cases, an oligonucleotide index tag may also be incorporated with adaptors attached to a target nucleic acid. In cases in which deletion products are generated from a plurality of polynucleotides prior to hybridizing the deletion products to a nucleic acid immobilized on a structure (e.g., a sensor element such as a particle), polynucleotides corresponding to different nucleic acids of interest may first be attached to different oligonucleotide tags such that subsequently generated deletion products corresponding to different nucleic acids of interest may be grouped or differentiated. Consequently, deletion products derived from the same nucleic acid of interest may have the same oligonucleotide index tag such that the index tag identifies sequencing reads derived from the same nucleic acid of interest. Likewise, deletion products derived from different nucleic acids of interest may have different oligonucleotide index tags to allow them to be grouped or differentiated such as on a sensor element. Oligonucleotide index tags may range in length from about 5, 10, 15, 20, 25, 30, 35, 40, 45, 50, 75, to 100 nucleotides or base pairs, or any length in between.

In some cases, the oligonucleotide index tags may be added separately or in conjunction with a primer, primer binding site or other component. Conversely, a pair-end read may be performed, wherein the read from the first end may comprise a portion of the sequence of interest and the read from the other (second) end may be utilized as a tag to identify the fragment from which the first read originated.

In some cases, a sequencing read may be initiated from the point of incorporation of the modified nucleotide into an extended capture probe. In some cases, a sequencing primer may be hybridized to extended capture probes or their complements, which may be optionally amplified prior to initiating a sequence read, and extended in the presence of natural nucleotides. In some cases, extension of the sequencing primer may stall at the point of incorporation of the first modified nucleotide incorporated in the template, and a complementary modified nucleotide may be incorporated at the point of stall using a polymerase capable of incorporating a modified nucleotide (e.g. TiTaq polymerase). In some cases, a sequencing read may be initiated at the first base after the stall or point of modified nucleotide incorporation. In a sequencing-by-synthesis method, a sequencing read may be initiated at the first base after the stall or point of modified nucleotide incorporation.

The present disclosure describes methods and compositions related to nucleic acid (polynucleotide) sequencing. Some methods of the present disclosure may provide for identification and quantification of nucleic acids in a subject or a sample. In some cases, the nucleotide sequence of a portion of a target nucleic acid or fragment thereof may be determined using a variety of methods and devices. Examples of sequencing methods include electrophoretic, sequencing by synthesis, sequencing by ligation, sequencing by hybridization, single-molecule sequencing, and real time sequencing methods. In some cases, the process to determine the nucleotide sequence of a target nucleic acid or fragment thereof may be an automated process. In some amplification reactions, capture probes may function as primers permitting the priming of a nucleotide synthesis reaction using a polynucleotide from the nucleic acid sample as a template. In this way, information regarding the sequence of the polynucleotides supplied to the array may be obtained. In some cases, polynucleotides hybridized to capture probes on the array may serve as sequencing templates if primers that hybridize to the polynucleotides bound to the capture probes and sequencing reagents are further supplied to the array.

Nucleic acid analysis methods may generate paired end reads on nucleic acid clusters. In some cases, a nucleic acid cluster may be immobilized on a sensor element, such as a surface. In some cases, paired end sequencing facilitates reading both the forward and reverse template strands of each cluster during one paired-end read. In some cases, template clusters may be amplified on the surface of a substrate (e.g. a flow-cell) by bridge amplification and sequenced by paired primers sequentially. Upon amplification of the template strands, a bridged double stranded structure may be produced. This may be treated to release a portion of one of the strands of each duplex from the surface. The single stranded nucleic acid may be available for sequencing, primer hybridization and cycles of primer extension. After the first sequencing run, the ends of the first single stranded template may be hybridized to the immobilized primers remaining from the initial cluster amplification procedure. The immobilized primers may be extended using the hybridized first single strand as a template to resynthesize the original double stranded structure. The double stranded structure may be treated to remove at least a portion of the first template strand to leave the resynthesized strand immobilized in single stranded form. The resynthesized strand may be sequenced to determine a second read, whose location originates from the opposite end of the original template fragment obtained from the fragmentation process.

Nucleic acid sequencing may be single-molecule sequencing or sequencing by synthesis. Sequencing may be massively parallel array sequencing (e.g., Illumina™ sequencing), which may be performed using template nucleic acid molecules immobilized on a support, such as a flow cell. A high-throughput sequencing method may sequence simultaneously (or substantially simultaneously) at least about 10,000, 100,000, 1 million, 10 million, 100 million, 1 billion, or more polynucleotide molecules. Sequencing methods may include, but are not limited to: pyrosequencing, sequencing-by synthesis, single-molecule sequencing, nanopore sequencing, semiconductor sequencing, sequencing-by-ligation, sequencing-by-hybridization, Digital Gene Expression (Helicos), massively parallel sequencing, e.g., Helicos, Clonal Single Molecule Array (Solexa/Illumina), sequencing using PacBio, SOLID, Ion Torrent, or Nanopore platforms. Sequencing may comprise a first-generation sequencing method, such as Maxam-Gilbert or Sanger sequencing, or a high-throughput sequencing (e.g., next-generation sequencing or NGS) method.

The sequencing methods of the present disclosure may be able to detect germline susceptibility loci, somatic single nucleotide polymorphisms (SNPs), small insertion and deletion (indel) mutations, copy number variations (CNVs) and structural variants (SVs).

Furthermore, the sequencing methods of the present disclosure may quantify a nucleic acid, thus allowing sequence variations within an individual sample may be identified and quantified (e.g., a first percent of a gene is unmutated and a second percent of a gene present in a sample contains an indel).

Nucleic acid analysis methods may comprise physical analysis of nucleic acids collected from a biological sample. A method may distinguish nucleic acids based on their mass, post-transcriptional modification state (e.g., capping), histonylation, circularization (e.g., to detect extrachromosomal circular DNA elements), or melting temperature. For example, an assay may comprise restriction fragment length polymorphism (RFLP) or electrophoretic analysis on DNA collected from a biological sample. In some cases, post-transcriptional modification may comprise 5′ capping, 3′ cleavage, 3′ polyadenylation, splicing, or any combination thereof.

Nucleic acid analysis may also include sequence-specific interrogation. An assay for sequence-specific interrogation may target a particular sequence to determine its presence, absence or relative abundance in a biological sample. For example, an assay may comprise a southern blot, qPCR, fluorescence in situ hybridization (FISH), array-Comparative Genomic Hybridization (array-CGH), quantitative fluorescence PCR (QF-PCR), nanopore sequencing, sequencing by hybridization, sequencing by synthesis, sequencing by ligation, or capture by nucleic acid binding moieties (e.g., single stranded nucleotides or nucleic acid binding proteins) to determine the presence of a gene of interest (e.g., an oncogene) in a sample collected from a subject. An assay may also couple sequence specific collection with sequencing analysis. For example, an assay may comprise generating a particular sticky-end motif in nucleic acids comprising a specific target sequence, ligating an adaptor to nucleic acids with the particular sticky-end motif, and sequencing the adaptor-ligated nucleic acids to determine the presence or prevalence of mutations in a gene of interest.

### Genomic Information

The present disclosure provides various systems and methods for analyzing (e.g., detecting or sequencing) nucleic acids. In some cases, genotypic (or genomic) information may be obtained using some of the compositions and methods of the present disclosure. In some cases, genotypic information can refer to information about substances comprising a nucleotide and/or a nucleic acid component. In some cases, genotypic information may comprise epigenetic information. In some cases, epigenetic information may comprise histone modification, DNA methylation, accessibility of different regions in a genome, dynamics changes thereof, or any combination thereof. In some cases, genotypic information may comprise primary structure information, secondary structure information, tertiary structure information, or quaternary information about a nucleic acid. In some cases, genotypic information may comprise information about nucleic acid-ligand interactions, wherein a ligand may comprise any one of various biological molecules and substances that may be found in living organisms, such as, nucleotides, nucleic acids, amino acids, peptides, proteins, monosaccharides, polysaccharides, lipids, phospholipids, hormones, or any combination thereof. In some cases, genotypic information may comprise a rate or prevalence of apoptosis of a healthy cell or a diseased cell. In some cases, genotypic information may comprise a state of a cell, such as a healthy state or a diseased state. In some cases, genotypic information may comprise chemical modification information of a nucleic acid molecule. In some cases, a chemical modification may comprise methylation, demethylation, amination, deamination, acetylation, oxidation, oxygenation, reduction, or any combination thereof. In some cases, genotypic information may comprise information regarding from which type of cell a biological sample originates. In some cases, genotypic information may comprise information about an untranslated region of nucleic acids.

In some cases, genotypic information may comprise information about a single cell, a tissue, an organ, a system of tissues and/or organs (such as cardiovascular, respiratory, digestive, or nervous systems), or an entire multicellular organism. In some cases, genotypic information may comprise information about an individual (e.g., an individual human being or an individual bacterium), or a population of individuals (e.g., human beings with diagnosed with cancer or a colony of bacteria). Genotypic information may comprise information from various forms of life, including forms of life from the Archaea, the Bacteria, the Eukarya, the Protozoa, the Chromista, the Plantae, the Fungi, or from the Animalia. In some cases, genotypic information may comprise information from viruses. In some cases, genotypic information may comprise information relating exons and introns in the code of life. In some cases, genotypic information may comprise information regarding variations or mutations in the primary structure of nucleic acids, including base substitutions, deletions, insertions, or any combination thereof. In some cases, genotypic information may comprise information regarding the inclusion of non-canonical nucleobases in nucleic acids. In some cases, genotypic information may comprise information regarding variations or mutations in epigenetics.

In some cases, genotypic information may comprise information regarding variations in the primary structure, variations in the secondary structure, variations in the tertiary structure, or variations in the quaternary structure of peptides and/or proteins that one or more nucleic acids encode.

In some cases, a genomic variant may be detected using an assay. In some cases, a genomic variant can refer to a nucleic acid sequence originating from a DNA address(es) in a sample that comprises a sequence that is different a nucleic acid sequence originating from the same DNA address(es) in a reference sample. In some cases, a genomic variant may comprise a mutation such as an insertion mutation, deletion mutations, substitution mutation, copy number variations, transversions, translocations, inversion, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, abnormal changes in nucleic acid methylation infection, chromosal lesions, DNA lesions, or any combination thereof. In some cases, a set of genomic variants may comprise a single nucleotide polymorphism (SNP).

### Dual Proteomic-Genomic Analysis

The present disclosure provides systems and methods for parallel identification of proteins and nucleic acids from a sample. In some cases, coupling these two forms of analysis can overcome limitations inherent to each type. In some cases, performing protein or nucleic acid analysis individually can generate indeterminate identifications, such as uncertain genomic copy numbers or inconclusive protein isoform assignments. In some cases, properly coupling nucleic acid and protein analysis can overcome these indeterminancies and can increase the level of diagnostic insight beyond the sum of what protein and nucleic acid analysis would provide individually.

In some cases, methods may comprise obtaining genomic data of a subject. In some cases, the genomic data of the subject may comprise whole genome sequencing data. In some cases, the genomic data of the subject may comprise exome sequencing data. In some cases, the genomic data of the subject may comprise transcriptome sequencing data. In some cases, the genomic data of the subject may comprise epigenome sequencing data. In some cases, the genomic data of the subject may comprise whole exome sequencing data, transcriptome sequencing data, epigenome sequencing data, or any combination thereof. In some cases, the genomic data of the subject may be retrieved from the subject's medical record. In some cases, the genomic data of the subject may be retrieved from a database.

In some cases, methods may comprise parallel collection of proteins and nucleic acids on a sensor element (e.g., a particle). For example, a method may comprise simultaneous adsorption of proteins and nucleic acids on a sensor element, followed by nucleic acid sequencing and protein analysis by mass spectrometry. In some cases, a method may also comprise simultaneous adsorption of proteins and nucleic acids on a sensor element and collection of the proteins and nucleic acids from the sensor element for parallel protein analysis (e.g., mass spectrometry or protein sequencing) and nucleic acid sequencing. In some cases, a method may comprise separation of the proteins from the nucleic acids, such as by chromatography, separate elution of the proteins and nucleic acids from a sensor element, differential precipitation, phase separation, or affinity capture. In some cases, a method may comprise adsorption of proteins on a sensor element, followed by collection of nucleic acids from the sample. In some cases, a method may comprise dividing a sample into separate portions for protein (e.g., biomolecule corona) and nucleic acid analysis.

In some cases, nucleic acid analysis may guide or inform protein (e.g., biomolecule corona) analysis. In some cases, the results of nucleic acid analysis may contribute to a protein identification. In some cases, protein analysis may determine whether a protein is present, and nucleic acid analysis may determine the exact sequence of the protein. In some cases, this can occur when mass spectrometric data identifies only a portion of a protein or peptide sequence. In some cases, nucleic acid data, such as the identification of a particular RNA isoform in a sample, may be used to discern the identity or full sequence of the protein or peptide. As an example, cases in which protein domain transpositions (e.g., an HRAS protein kinase domain transpositions leading to constitutive activity and possible increased cancer risk) do not alter peptide fragment digestion patterns can be difficult to ascertain through protein analysis alone, but may be elucidated by a combination of proteomic analysis and genomic analysis, wherein the proteomic analysis may identify the presence of the protein, and genomic analysis can determine its transposition state.

In some cases, nucleic acid (e.g., transcriptomic) analysis may be used to determine which protein splicing variants are present in a sample. In some cases, RNA analysis may further be used to determine the relative abundances of the protein splicing variants. In some cases, protein analysis may be used to determine the RNA variants (e.g., mRNA splicing variants) present in a sample.

In some cases, nucleic acid analysis may also distinguish an individual protein from among an experimentally identified protein group. In some cases, protein analysis may identify protein groups comprising pluralities of proteins. In some cases, nucleic acid information such as a genomic sequence, an RNA sequence (e.g., a particular RNA isoform or splicing variant), or expression modulating nucleic acid modification (e.g., methylation) may be used to discern the protein or set of proteins that are present from among the protein group. For example, protein analysis may identify a protein group consisting of seven related proteins (e.g., the seven confirmed 14-3-3 protein isoforms found in mammalian cells), while subsequent nucleic acid analysis may determine that RNA encoding two of the seven related proteins are present in the sample, thereby determining the proteins from among the protein group present in the sample.

In some cases, nucleic acid analysis may increase the number of proteins or protein groups identified by a protein assay. In some cases, nucleic acid analysis may determine the particular proteins present within an identified protein group, or may identify protein subgroups from among a protein group. In some cases, coupling nucleic acid analysis with protein analysis may thus increase the number of identified proteins or protein groups by at least 5%, at least 10%, at least 15%, at least 20%, at least 25%, at least 30%, at least 40%, at least 50%, at least 60%, at least 80%, or at least 100% relative to an assay comprising protein analysis only.

In some cases, nucleic acid analysis may also guide protein (e.g., protein corona) and biomolecule corona analysis. In some cases, mass spectrometric analysis (and thereby a biomolecule corona method) comprises data-dependent acquisition, in which a number of ions (e.g., particular m/z ratios) are pre-selected for tandem mass spectrometric analysis. An ion or plurality of ions of the data-dependent acquisition may be selected based on nucleic acid analysis results. For example, nucleic acid analysis may identify two protein variants with predicted peptide fragments that share a mass but vary in sequence and provide instructions to a mass spectrometric instrument to include the mass of the peptide fragment in a data-dependent acquisition. Mass spectrometric analysis may also comprise data-independent acquisition, in which a mass/charge range is preselected for tandem mass spectrometric analysis. In such cases, nucleic acid analysis may dictate or partially dictate the mass/charge ranges analyzed. Nucleic acid analysis may also guide ionization methodology. For example, results from nucleic acid analysis may determine laser power for a matrix assisted laser desorption/ionization (MALDI) mass spectrometric experiment, and thereby affect the biomolecule fragments generated for analysis.

### Subject-Specific Libraries

In some cases, nucleic acid and protein analysis may be used individually or in combination to develop subject-specific (e.g., patient-specific) libraries that can expedite and expand the depth and accuracy of mass spectrometric analyses. In some cases, mass spectrometric analyses may be limited by degrees of ambiguity in protein assignments. In some cases, only a portion of a protein's sequence may be covered by mass spectrometric signals, thereby rendering the assay blind to variations in the remaining unsequenced portion. In some cases, mass spectrometric analysis can be incapable of identifying particular transpositions (e.g., domain transpositions) and splicing variations. In some cases, rectifying such shortcomings can be expensive and time consuming. For example, expanding mass spectrometric assays to include multiple forms of digestions can increase sequence coverage at the expense of increased user input.

In some cases, generating a subject-specific library can allow faster and deeper analysis of mass spectrometric data from the subject. In some cases, a subject-specific library may comprise proteins present in a subject. In some cases, a subject-specific library may comprise nucleic acids (e.g., genes) present in a subject. In some cases, a subject-specific library may be used to generate a specific spectrum library comprising predicted experimental signals (e.g., mass spectrometric signals corresponding to peptide fragments or DNA electrophoresis bands) from the subject. In some cases, a subject-specific library may be generated with proteomic data, nucleic acid data, metabolomic data (e.g., measuring lactose hydrolysis to determine the presence of lactase), lipidomic data, or any combination thereof.

In some cases, a subject-specific library may increase the precision of protein or nucleic acid identifications. In some cases, possible protein identifications may be limited to potential protein sequences identified in a subject's genome. For example, a protein group encompassing 8 allelic variants may be narrowed to a specific form based on nucleic acid data from a subject.

In some cases, a subject-specific library can be constructed from nucleic acid data. In some cases, the data may be processed to identify sequence variants (e.g., based at least on alignment with a reference sequence), leading to a library of subject-specific nucleic acid variants. In some cases, the nucleic acid data may be derived from comprise whole genome sequencing or targeted sequencing using a specific or enriched portion of a genome or transcriptome. In some cases, the screening may comprise exome sequencing to thereby identify splicing variants from a sample.

In some cases, nucleic acid sequences (e.g., gene variants) may be translated in-silico to generate a subject-specific protein sequence database. In some cases, a database may comprise protein sequences which may aid in protein or protein group identifications from mass spectrometric data on a sample. In some cases, the database may be used to determine which proteins from among a protein group are present in a sample. In some cases, the database may also comprise abundances or relative abundances of protein sequences. In some cases, the database may comprise the relative abundances of different isoforms of a protein in a sample or the mutation rate for a gene or among multiple genes.

In some cases, the subject-specific protein sequence database may be used to computationally generate subject-specific spectrum libraries, which may comprise expected or putative mass spectrometric signals from samples from the subject. In some cases, the computational prediction of mass spectrometric features may account for experimental variables, such as sample purification and digestion methods. In some cases, the subject-specific spectrum library may comprise expected tandem mass spectrometric features, as well as predicted relative intensities of mass spectrometric features. In some cases, the subject-specific spectrum library may also comprise empirically derived mass spectrometric features. For example, peptide variants may be identified from data-dependent acquisition mass spectrometric experiments.

In some cases, the subject-specific spectrum library may be used to deconvolute mass spectrometric data (e.g., data-independent acquisition mass spectrometric data) collected from samples from the subject, and to thus identify particular genomic variants in a sample. In some cases, the subject-specific spectrum library can overcome this limitation (when present) by correlating mass spectrometric features with known proteins or protein variants, in some cases allowing the mass spectrometric data to be used to identify partial or complete protein sequences. In some cases, the subject-specific spectrum library can aid in quantifying (e.g., determining the abundance in the subject sample) proteins from mass spectrometric data. In some cases, this in part may comprise apportioning a common mass spectrometric signal (e.g., an m/z common to multiple proteins) between multiple proteins identified in a sample.

In some cases, a utility of subject-specific libraries is that they may differentiate and enable the identification of proteins from groups (e.g., protein groups) that are difficult to distinguish solely through protein analysis. In some cases, the subject-specific library can also enable relative or absolute quantification (e.g., concentration in a biological sample) of a protein or set of proteins. In some cases, a subject-specific library can also determine the presence of mutations, such as point mutations or transpositions, which may not be detectable through protein analysis (e.g., mass spectrometry) alone.

In some cases, heterozygous pairs can be particularly difficult to detect through mass spectrometric analysis alone. In some cases, the distinct points or regions of a heterozygous pair may not be detected during protein analysis. For example, mass spectrometric analysis might not produce signals covering the region or regions that differ between proteins arising from multiple alleles. In some cases, pairing nucleic acid analysis can determine whether a subject is homozygous or heterozygous for a particular gene, and can further determine the allele or alleles that are present.

In some cases, nucleic acid sequences obtained for the subject may be translated in silico to construct a subject-specific protein sequence database containing predicted protein sequences present in the subject. In some cases, various proteoforms may be predicted for a single gene, such as in the case of heterozygosity or alternative splicing. In some cases, the protein sequences may be used to generate predicted mass spectrometric signals from a subject sample. In some cases, this can simplify the analysis of a protein mass spectrometry data from a subject and enhance its specificity and accuracy as well. For example, where a set of mass spectrometric signals identifies a protein group from a sample, tandem nucleic acid sequences and mass spectrometric signals may identify a particular protein or set of proteins present in the sample, such as a pair of proteins arising from two alleles for a gene. In some cases, the protein sequences may be used to generate predicted peptide sequences digested from a subject sample. In some cases, this can simplify the analysis of a protein sequencing data from a subject and enhance its specificity and accuracy as well.

In some cases, protein data may be used to determine expression levels in a subject. While nucleic acid analysis may identify a number of genes present in a subject, protein analysis on samples from the subject can determine which genes are being expressed and translated.

### Non-Specific Binding

A surface may bind biomolecules through variably selective adsorption (e.g., adsorption of biomolecules or biomolecule groups upon contacting the particle to a biological sample comprising the biomolecules or biomolecule groups, which adsorption is variably selective depending upon factors including e.g., physicochemical properties of the particle) or non-specific binding. Non-specific binding can refer to a class of binding interactions that exclude specific binding. Examples of specific binding may comprise protein-ligand binding interactions, antigen-antibody binding interactions, nucleic acid hybridizations, or a binding interaction between a template molecule and a target molecule wherein the template molecule provides a sequence or a 3D structure that favors the binding of a target molecule that comprise a complementary sequence or a complementary 3D structure, and disfavors the binding of a non-target molecule(s) that does not comprise the complementary sequence or the complementary 3D structure.

Non-specific binding may comprise one or a combination of a wide variety of chemical and physical interactions and effects. Non-specific binding may comprise electromagnetic forces, such as electrostatics interactions, London dispersion, Van der Waals interactions, or dipole-dipole interactions (e.g., between both permanent dipoles and induced dipoles). Non-specific binding may be mediated through covalent bonds, such as disulfide bridges. Non-specific binding may be mediated through hydrogen bonds. Non-specific binding may comprise solvophobic effects (e.g., hydrophobic effect), wherein one object is repelled by a solvent environment and is forced to the boundaries of the solvent, such as the surface of another object. Non-specific binding may comprise entropic effects, such as in depletion forces, or raising of the thermal energy above a critical solution temperature (e.g., a lower critical solution temperature). Non-specific binding may comprise kinetic effects, wherein one binding molecule may have faster binding kinetics than another binding molecule.

Non-specific binding may comprise a plurality of non-specific binding affinities for a plurality of targets (e.g., at least 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000, 20,000, 30,000, 40,000, 50,000 different targets adsorbed to a single particle). The plurality of targets may have similar non-specific binding affinities that are within about one, two, or three magnitudes (e.g., as measured by non-specific binding free energy, equilibrium constants, competitive adsorption, etc.). This may be contrasted with specific binding, which may comprise a higher binding affinity for a given target molecule than non-target molecules.

Biomolecules may adsorb onto a surface through non-specific binding on a surface at various densities. In some cases, biomolecules or proteins may adsorb at a density of at least about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at least about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm². In some cases, biomolecules or proteins may adsorb at a density of at most about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000 fg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 pg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 ng/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 μg/mm². In some cases, biomolecules or proteins may adsorb at a density of at most about 1, 2, 3, 4, 5, 6, 7, 8, 9, 10, 20, 30, 40, 50, 60, 70, 80, 90, 100, 200, 300, 400, 500, 600, 700, 800, 900, or 1000 mg/mm².

Adsorbed biomolecules may comprise various types of proteins. In some cases, adsorbed proteins may comprise at least about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins. In some cases, adsorbed proteins may comprise at most about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10000 types of proteins.

In some cases, proteins in a biological sample may comprise at least about 1, 2, 3, 4, 5, 6, 7, 8, 9, 10, 15, 20, 25, or 30 orders of magnitudes in concentration. In some cases, proteins in a biological sample may comprise at most about 1, 2, 3, 4, 5, 6, 7, 8, 9, 10, 15, 20, 25, or 30 orders of magnitudes in concentration.

### Composition Improving Assay

In some cases, a method of the present disclosure may comprise using a composition improving assay. In some cases, an untargeted assay may be a composition improving assay. In some cases, a composition improving assay may improve access to a subset of biomolecules in a biological sample. In some cases, a composition improving assay may improve detection to a subset of biomolecules in a biological sample. In some cases, a composition improving assay may improve identification to a subset of biomolecules in a biological sample. In some cases, the subset of biomolecules may be low-abundance biomolecules. In some cases, the subset of biomolecules may be rare biomolecules. In some cases, a dynamic range of a biological sample may be compressed using a composition improving assay. In some cases, a dynamic range may be compressed by at least about 1, 2, 3, 4, 5, 6, 7, 8, 9, 10, 11, 12, 13, 14, or 15 magnitudes.

In some cases, the composition improving assay may comprise providing one or more of surface regions comprising one or more surface types. In some cases, the composition improving assay may comprise contacting the biological sample with the one or more surface regions to yield a set of adsorbed biomolecules on the one or more surface regions. In some cases, the composition improving assay may comprise desorbing, from the one or more surface regions, at least a portion of the set of adsorbed biomolecules to yield the set of polyamino acids. In some cases, the composition improving assay may comprise contacting the biological sample with the one or more surface regions to capture a set of biomolecules on the one or more surface regions. In some cases, the composition improving assay may comprise releasing, from the one or more surface regions, at least a portion of the set of biomolecules to yield the set of polyamino acids. In some cases, the one or more surface regions are disposed on a single continuous surface. In some cases, the one or more surface regions are disposed on one or more discrete surfaces. In some cases, the one or more discrete surfaces are surfaces of one or more particles. In some cases, the one or more particles may comprise a nanoparticle. In some cases, the one or more particles may comprise a microparticle. In some cases, the one or more particles may comprise a porous particle. In some cases, the one or more particles may comprise a bifunctional, trifunctional, or N-functional particle.

In some cases, the composition improving assay may comprise providing a plurality of surface regions comprising a plurality of surface types. In some cases, the composition improving assay may comprise contacting the biological sample with the plurality of surface regions to yield a set of adsorbed biomolecules on the plurality of surface regions. In some cases, the composition improving assay may comprise desorbing, from the plurality of surface regions, at least a portion of the set of adsorbed biomolecules to yield the set of polyamino acids. In some cases, the composition improving assay may comprise contacting the biological sample with the plurality of surface regions to capture a set of biomolecules on the plurality of surface regions. In some cases, the composition improving assay may comprise releasing, from the plurality of surface regions, at least a portion of the set of biomolecules to yield the set of polyamino acids. In some cases, the plurality of surface regions are disposed on a single continuous surface. In some cases, the plurality of surface regions are disposed on a plurality of discrete surfaces. In some cases, the plurality of discrete surfaces are surfaces of a plurality of particles. In some cases, the plurality of particles may comprise a nanoparticle. In some cases, the plurality of particles may comprise a microparticle. In some cases, the plurality of particles may comprise a porous particle. In some cases, the plurality of particles may comprise a bifunctional, trifunctional, or N-functional particle.

### Classification Using Machine Learning

In some cases, identifications of biomolecules may be processed using a machine learning algorithm. In some cases, the identifications of biomolecules may comprise identifications of nucleic acids, variants thereof, proteins, variants thereof, and any combination thereof. In some cases, the machine learning algorithm may be an unsupervised or self-supervised learning algorithm. In some cases, the machine learning algorithm may be trained to learn a latent representation of the identifications of the biomolecules. In some cases, the machine learning algorithm may be supervised learning algorithm. In some cases, the machine learning algorithm may be trained to learn to associate a given set of identifications with a value associated with a predetermined task. For example, the predetermined task may comprise determining a disease state associated with the given set of identifications, where the value may indicate the probability of the disease state being present in a subject associated with the given set of identifications.

In some cases, the method of determining a set of biomolecules associated with the disease or disorder and/or disease state can include the analysis of the biomolecule corona of at least two samples. This determination, analysis or statistical classification can be performed by methods, including, but not limited to, for example, a wide variety of supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive Bayes, linear regression, polynomial regression, SVM for regression, K-means clustering, and hidden Markov models, among others. In other words, the biomolecules in the corona of each sample are compared/analyzed with each other to determine with statistical significance what patterns are common between the individual corona to determine a set of biomolecules that is associated with the disease or disorder or disease state.

In some cases, machine learning algorithms can be used to construct models that accurately assign class labels to examples based on the input features that describe the example.

In some case it may be advantageous to employ machine learning and/or deep learning approaches for the methods described herein. For example, machine learning can be used to associate the biomolecule corona with various disease states (e.g. no disease, precursor to a disease, having early or late stage of the disease, etc.). For example, in some cases, one or more machine learning algorithms can be employed in connection with the methods disclosed hereinto analyze data detected and obtained by the biomolecule corona and sets of biomolecules derived therefrom. For example, machine learning can be coupled with genomic and proteomic information obtained using the methods described herein to determine not only if a subject has a pre-stage of cancer, cancer or does not have or develop cancer, and also to distinguish the type of cancer.

In some cases, machine learning algorithms may also be used to associate the results from protein corona analysis and results from nucleic acid sequencing analysis and further associate any trends or correlations between proteins and nucleic acids to a biological state (e.g., disease state, health state, subtypes of disease such as stages of disease are cancer subtypes).

In some cases, machine learning may be used to cluster proteins detected using a plurality of surfaces. In some cases, a panel of surfaces may be used to assay proteins from one or more biological samples. In some cases, a surface in the panel of surfaces may comprise diverse physicochemical properties. In some cases, proteins detected by the panel of surfaces may be clustered using a clustering algorithm. In some cases, proteins detected by the panel of surfaces may be clustered based at least partially on the intensities of detected protein signals, particle chemical properties, protein structural and/or functional groups, or any combination thereof.

A panel of surfaces may comprise any number of surfaces. In some cases, a panel of surfaces may comprise at least about 1, 2, 3, 4, 5, 6, 7, 8, 9, 10, 20, 30, 40, 50, 60, 70, 80, 90, 100, 200, 300, 400, 500, 600, 700, 800, 900, or 1000 surfaces. In some cases, a panel of surfaces may comprise at most about 1, 2, 3, 4, 5, 6, 7, 8, 9, 10, 20, 30, 40, 50, 60, 70, 80, 90, 100, 200, 300, 400, 500, 600, 700, 800, 900, or 1000 surfaces.

Inputs to a machine learning algorithm may comprise various kinds of inputs. In some cases, an input may comprise a value that represents a physicochemical property of a surface used to assay a biomolecule. A physicochemical property of a particle may comprise various properties disclosed herein, which includes: charge, hydrophobicity, hydrophilicity, amphipathicity, coordinating, reaction class, surface free energy, various functional groups/modifications (e.g., sugar, polymer, amine, amide, epoxy, crosslinker, hydroxyl, aromatic, or phosphate groups). In some cases, an input may comprise a value that represents a parameter of a given assay. A parameter may comprise incubation conditions including temperature, incubation time, pH, buffer type, and any variables in performing an assay disclosed herein.

In some cases, a clustering algorithm can refer to a method of grouping samples in a dataset by some measure of similarity. In some cases, samples can be grouped in a set space, for example, element ‘a’ is in set ‘A’. In some cases, samples can be grouped in a continuous space, for example, element ‘a’ is a point in Euclidean space with distance ‘l’ away from the centroid of elements comprising cluster ‘A’. In some cases, samples can be grouped in a graph space, for example, element ‘a’ is highly connected to elements comprising cluster ‘A’. In some cases, clustering can refer to the principle of organizing a plurality of elements into groups in some mathematical space based on some measure of similarity.

In some cases, clustering can comprise grouping any number of biomolecules in a dataset by any quantitative measure of similarity. In some cases, clustering can comprise K-means clustering. In some cases, clustering can comprise hierarchical clustering. In some cases, clustering can comprise using random forest models. In some cases, clustering can comprise boosted tree models. In some cases, clustering can comprise using support vector machines. In some cases, clustering can comprise calculating one or more N−1 dimensional surfaces in N-dimensional space that partitions a dataset into clusters. In some cases, clustering can comprise distribution-based clustering. In some cases, clustering can comprise fitting a plurality of prior distributions over the data distributed in N-dimensional space. In some cases, clustering can comprise using density-based clustering. In some cases, clustering can comprise using fuzzy clustering. In some cases, clustering can comprise computing probability values of a data point belonging to a cluster. In some cases, clustering can comprise using constraints. In some cases, clustering can comprise using supervised learning. In some embodiments, clustering can comprise using unsupervised learning.

In some cases, clustering can comprise grouping biomolecules based on similarity. In some cases, clustering can comprise grouping biomolecules based on quantitative similarity. In some cases, clustering can comprise grouping biomolecules based on one or more features of each protein. In some cases, clustering can comprise grouping biomolecules based on one or more labels of each protein. In some cases, clustering can comprise grouping biomolecules based on Euclidean coordinates in a numerical representation of biomolecules. In some cases, clustering can comprise grouping biomolecules based on protein structural groups or functional groups (e.g., protein structures, substructures, or functional groups from protein databases such as Protein Data Bank or CATH Protein Structure Classification database). In some cases, a protein structural group or functional group may comprise protein primary structure, secondary structure, tertiary structure, or quaternary structure. In some cases, a protein structural group or functional group may be based at least partially on alpha helices, beta sheets, relative distribution of amino acids with different properties (e.g., aliphatic, aromatic, hydrophilic, acidic, basic, etc.), a structural families (e.g., TIM barrel and beta barrel fold), protein domains (e.g., Death effector domain). In some cases, a protein structural group or functional group may be based at least partially on functional or spatial properties (e.g., functional groups-group of immune globulins, cytokines, cytoskeletal biomolecules, etc.).

### Computer Systems

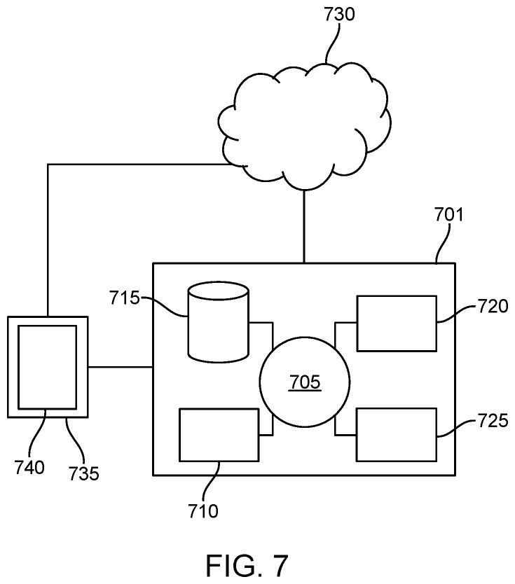

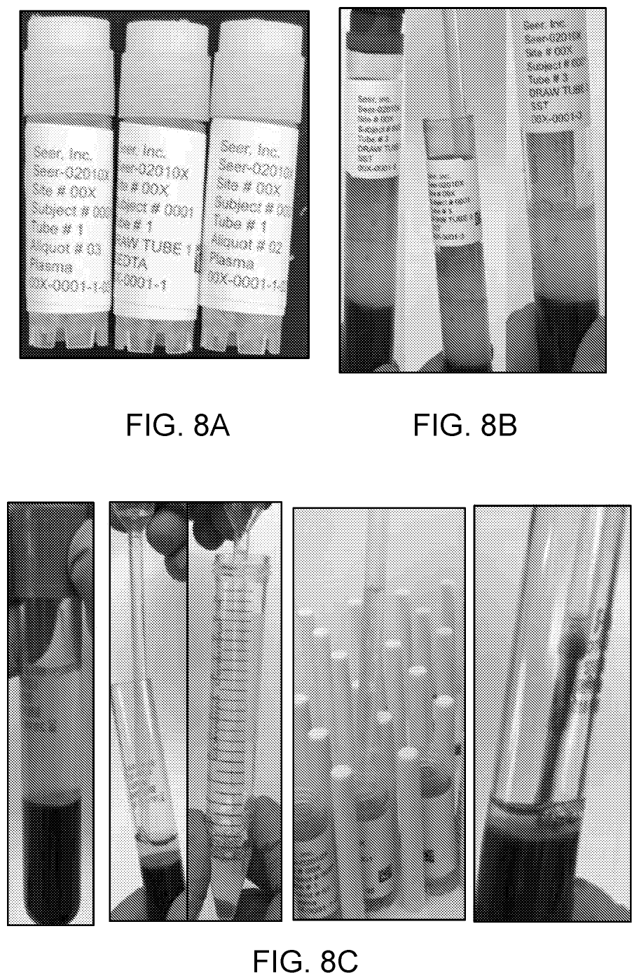

The present disclosure provides computer systems that are programmed to implement methods of the disclosure. FIG. 7 shows a computer system 701 that is programmed or otherwise configured to, for example, assay a set of nucleic acids, generate a set of expressible proteoforms, assay a set of polyamino acids, generate proteomic information, map a set of identifications to a set of expressible proteoforms, determine a set of expressed proteoforms, determine expression levels of one or more regions in one or more nucleic acid sequences, or perform any one of the methods disclosed herein or steps thereof.

The computer system 701 may regulate various aspects of analysis, calculation, and generation of the present disclosure, such as, for example, assaying a set of nucleic acids, generating a set of expressible proteoforms, assaying a set of polyamino acids, generating proteomic information, mapping a set of identifications to a set of expressible proteoforms, determining a set of expressed proteoforms, determining expression levels of one or more regions in one or more nucleic acid sequences, or performing any one of the methods disclosed herein or steps thereof. The computer system 701 may be an electronic device of a user or a computer system that is remotely located with respect to the electronic device. The electronic device may be a mobile electronic device.

The computer system 701 includes a central processing unit (CPU, also “processor” and “computer processor” herein) 705, which may be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 may be a data storage unit (or data repository) for storing data. The computer system 701 may be operatively coupled to a computer network (“network”) 730 with the aid of the communication interface 720. The network 730 may be the Internet, an internet and/or extranet, or an intranet and/or extranet that is in communication with the Internet.

The network 730 in some cases is a telecommunication and/or data network. The network 730 may include one or more computer servers, which may enable distributed computing, such as cloud computing. For example, one or more computer servers may enable cloud computing over the network 730 (“the cloud”) to perform various aspects of analysis, calculation, and generation of the present disclosure, such as, for example, assaying a set of nucleic acids, generating a set of expressible proteoforms, assaying a set of polyamino acids, generating proteomic information, mapping a set of identifications to a set of expressible proteoforms, determining a set of expressed proteoforms, determining expression levels of one or more regions in one or more nucleic acid sequences, or performing any one of the methods disclosed herein or steps thereof. Such cloud computing may be provided by cloud computing platforms such as, for example, Amazon Web Services (AWS), Microsoft Azure, Google Cloud Platform, and IBM cloud. The network 730, in some cases with the aid of the computer system 701, may implement a peer-to-peer network, which may enable devices coupled to the computer system 701 to behave as a client or a server.

The CPU 705 may comprise one or more computer processors and/or one or more graphics processing units (GPUs). The CPU 705 may execute a sequence of machine-readable instructions, which may be embodied in a program or software. The instructions may be stored in a memory location, such as the memory 710. The instructions may be directed to the CPU 705, which may subsequently program or otherwise configure the CPU 705 to implement methods of the present disclosure. Examples of operations performed by the CPU 705 may include fetch, decode, execute, and writeback.

The CPU 705 may be part of a circuit, such as an integrated circuit. One or more other components of the system 701 may be included in the circuit. In some cases, the circuit is an application specific integrated circuit (ASIC).

The storage unit 715 may store files, such as drivers, libraries and saved programs. The storage unit 715 may store user data, e.g., user preferences and user programs. The computer system 701 in some cases may include one or more additional data storage units that are external to the computer system 701, such as located on a remote server that is in communication with the computer system 701 through an intranet or the Internet.

The computer system 701 may communicate with one or more remote computer systems through the network 730. For instance, the computer system 701 may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 701 via the network 730.

Methods as described herein may be implemented by way of machine (e.g., computer processor) executable code stored on an electronic storage location of the computer system 701, such as, for example, on the memory 710 or electronic storage unit 715. The machine executable or machine readable code may be provided in the form of software. During use, the code may be executed by the processor 705. In some cases, the code may be retrieved from the storage unit 715 and stored on the memory 710 for ready access by the processor 705. In some situations, the electronic storage unit 715 may be precluded, and machine-executable instructions are stored on memory 710.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 701, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 may include or be in communication with an electronic display 735 that comprises a user interface (UI) 740 for assaying a set of nucleic acids, generating a set of expressible proteoforms, assaying a set of polyamino acids, generating proteomic information, mapping a set of identifications to a set of expressible proteoforms, determining a set of expressed proteoforms, determining expression levels of one or more regions in one or more nucleic acid sequences, or performing any one of the methods disclosed herein or steps thereof. Examples of UIs include, without limitation, a graphical user interface (GUI) and web-based user interface.

Methods and systems of the present disclosure may be implemented by way of one or more algorithms. An algorithm may be implemented by way of software upon execution by the central processing unit 705. The algorithm can, for example, assay a set of nucleic acids, generate a set of expressible proteoforms, assay a set of polyamino acids, generate proteomic information, map a set of identifications to a set of expressible proteoforms, determine a set of expressed proteoforms, determine expression levels of one or more regions in one or more nucleic acid sequences, or perform any one of the methods disclosed herein or steps thereof.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Peptide-Centric Analyses of Human Plasma

This example illustrates methods and systems for analyzing differences between protein isoforms. Protein isoforms from plasma samples of 80 healthy controls and 61 patients with early-stage non-small-cell lung cancer (NSCLC) were analyzed using a method of the present disclosure. Processing the 141 plasma samples with the method yielded 22,993 peptides corresponding to 2,569 protein groups at a confidence of 1% false discovery rate. Four proteins with peptides with significant abundance differences (p<0.05; Benjamini-Hochberg corrected) were extracted in healthy control and cancer plasma samples. For one, the abundance variation can be explained by underlying annotated protein isoforms. For a second, evidence was found for differentially transcribed isoforms in the broader sequence data, but not in the known annotated protein isoforms. The others may be explained by novel isoforms or post-translational modifications. In addition, protein variants arising from allelic variation were identified. Whole exome sequencing was performed on buffy coat samples from 29 individuals in the NSCLC study (plasma samples may also be used). Then, personalized mass spectrometry search databases were created for each individual subject from the exome sequences. From these databases, 422 protein variants were identified, where some are related to lung cancer. The results demonstrate that some methods of the present disclosure can generate plasma proteome profiles that enable identification of proteoforms present in plasma at a scale sufficient to enable population-scale proteomic studies powered to reveal novel mechanistic and biomedical insights.

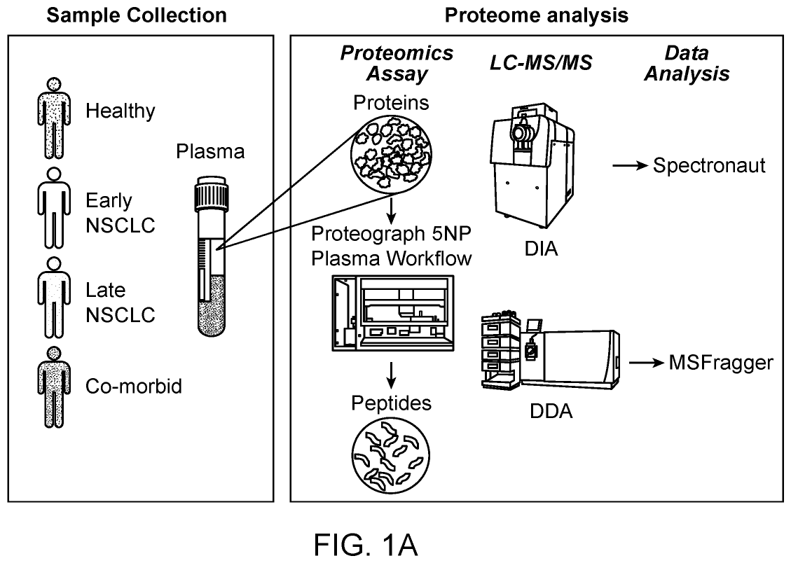

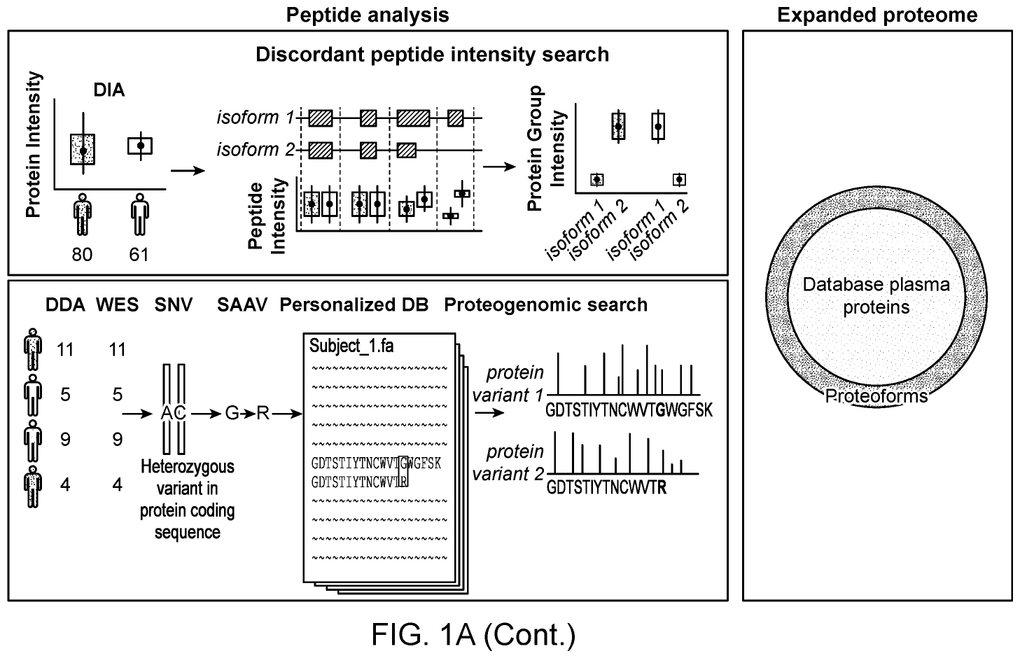

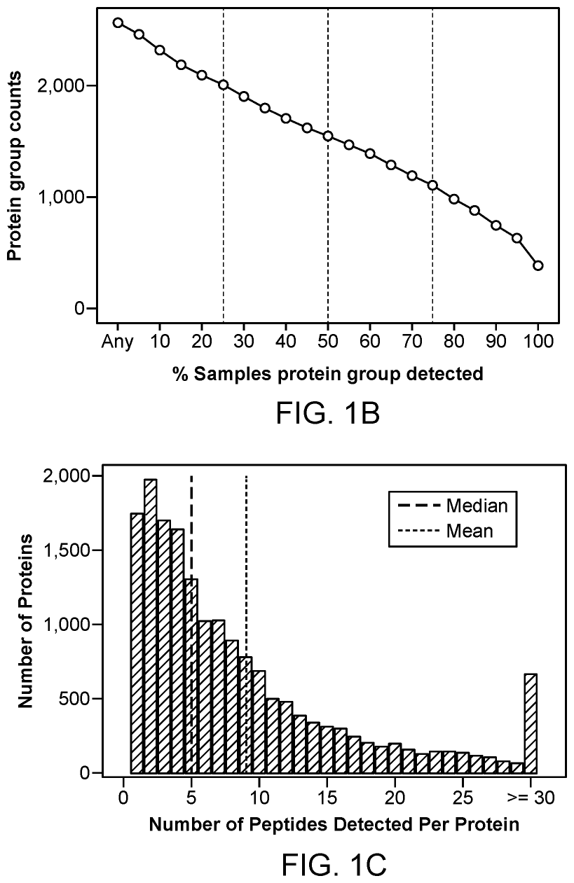

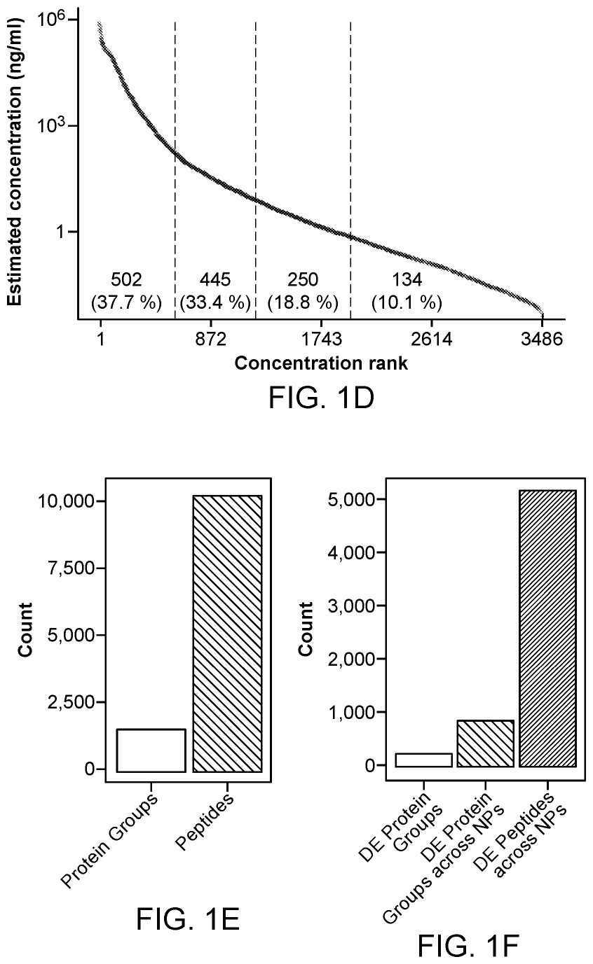

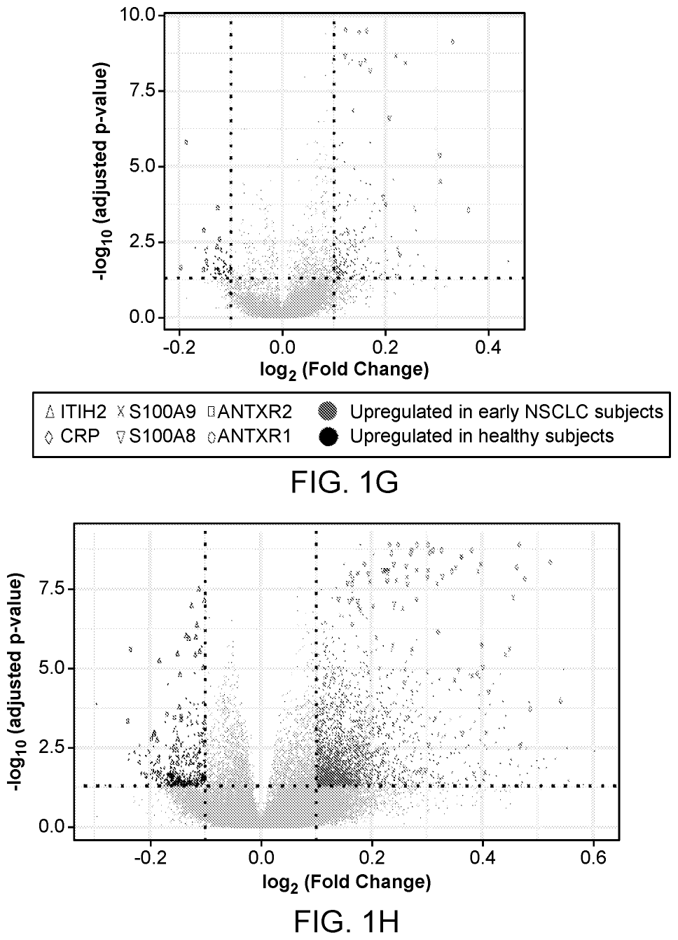

The proteomes of healthy individuals (individuals with early and late NSCLC, and individuals with comorbidities) were analyzed using PROTEOGRAPH™ to explore the ability to infer proteoforms. In data independent acquisition (DIA) data generated from 141 subjects (80 healthy subjects and 61 subjects identified as having early NSCLC), a discordant peptide intensity search was used (FIG. 1A) to infer four proteins with differentially abundant protein isoforms, including BMP1, which plays both an activator and repressor role in cancer. Further, in data dependent acquisition data generated from 29 subjects (11 healthy subjects, 5 early NSCLC subjects, 9 late NSCLC subjects, and 4 subjects with comorbidities), a proteogenomic search was used (FIG. 1A), which incorporated WES data to generate personalized databases, to identify 422 protein variants. The results demonstrate that (i) PROTEOGRAPH™-based proteomic data can be used to identify proteoforms and (ii) proteoform inference is enabled by proteomic data obtained using PROTEOGRAPH™

### Peptide-Level Analyses Provides Unique Biological Insight at the Peptide-Level Versus Protein-Level

To assess the ability to detect proteoforms (protein isoforms and protein variants) in LC-MS/MS-based plasma proteomic data derived from PROTEOGRAPH™, DIA data was generated from PROTEOGRAPH™ performed on 141 subjects (80 healthy subjects and 61 subjects identified as having early NSCLC, hereto referred to as “early NSCLC subjects”) using 10 physiochemically distinct nanoparticles (NP) (FIG. 1A). Data was analyzed using Spectronaut™ (Biognosys, Switzerland) for peptide identification and protein group assembly. Across the 141 samples, 2,569 unique protein groups were detected and 2,010 protein groups were identified in at least 25% of subjects (FIG. 1B). Mapping to the 2,569 unique protein groups, 22,993 peptides were identified, with a median of 5 peptides per protein group and mean of 8.8 peptides per protein group (FIG. 1C). To examine the extent to which these protein groups cover the broad dynamic range of the human plasma proteome, the 2,569 protein groups were ranked using protein abundances derived from the Human Plasma Proteome Project (HPPP) (FIG. 1D). Ranking the protein groups by the corresponding protein abundances in the HPPP reference from lowest to highest, the first quartile, median, and third quartile were observed at 0.785, 8.8, and 190 ng/ml, respectively. On the other hand, when protein groups found in a depleted plasma approach was ranked by abundance in the HPPP reference, the first quartile, median, and third quartile were observed at 27, 190, and 2,600 ng/ml, respectively. The results demonstrate that PROTEOGRAPH™ can extract lower abundance protein groups. Further, these results indicate that PROTEOGRAPH™-derived proteins have suitable depth and diversity to perform proteoform discovery analyses.

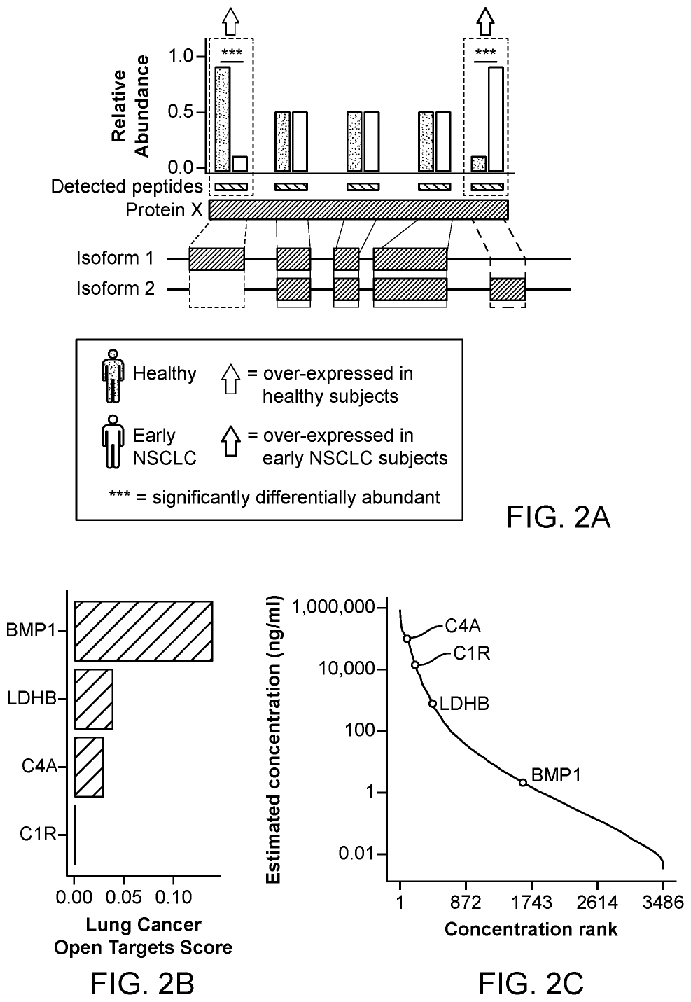

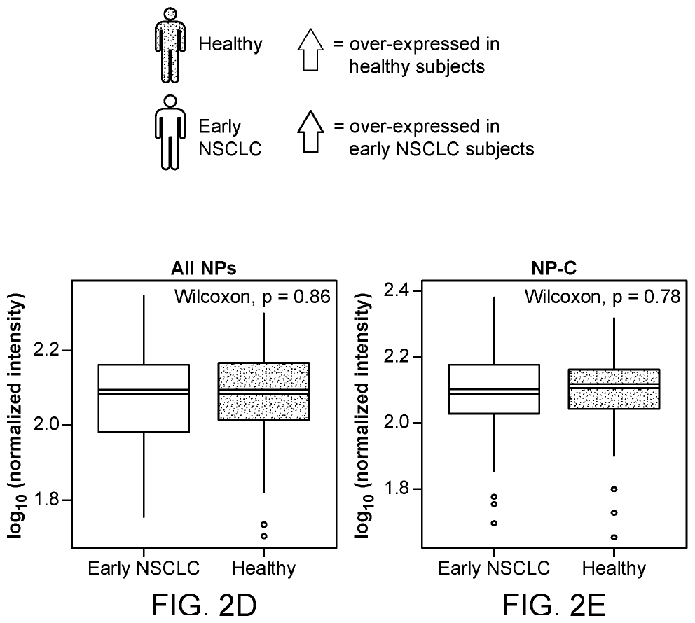

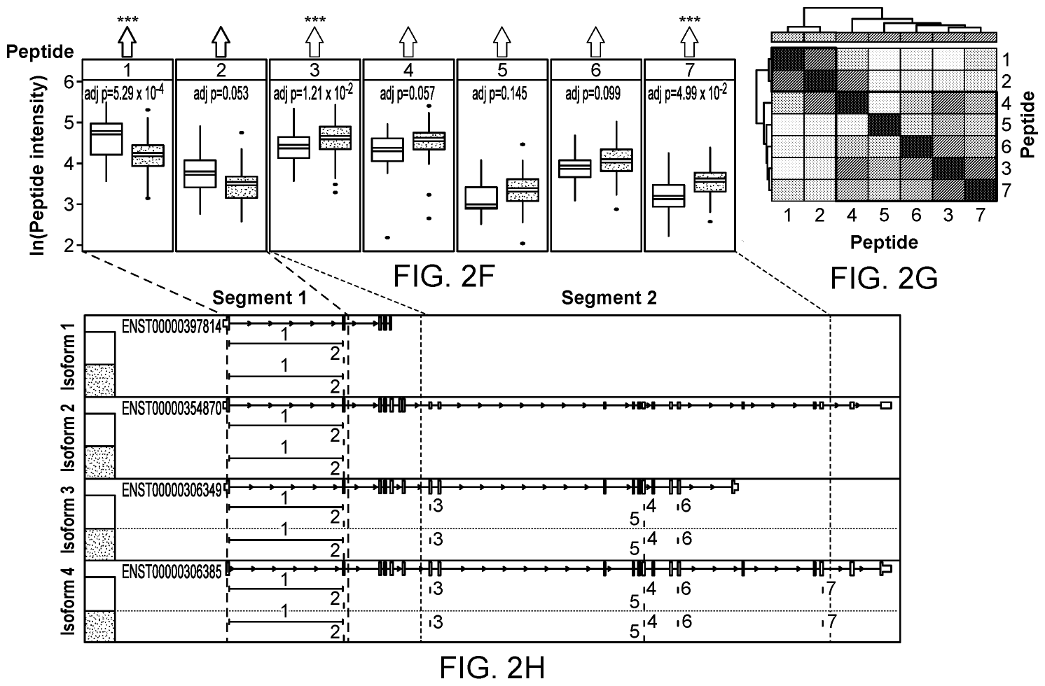

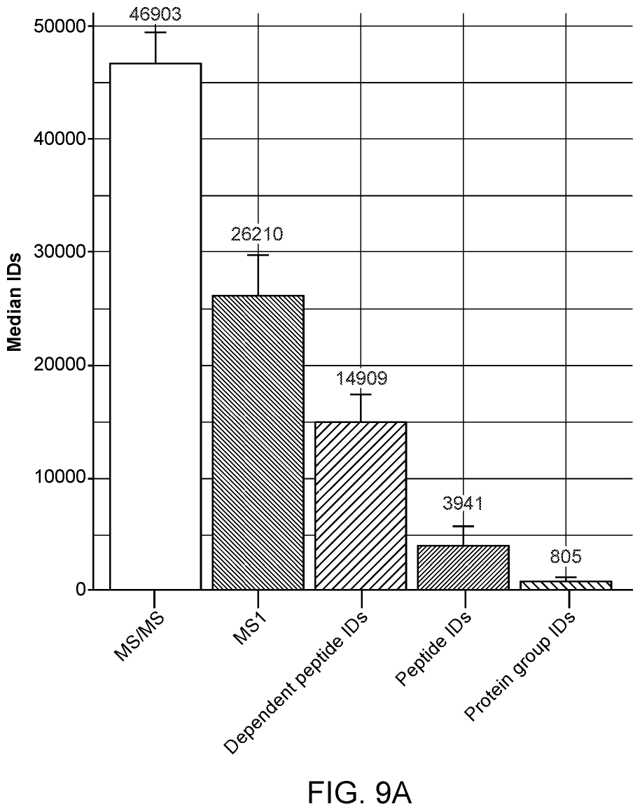

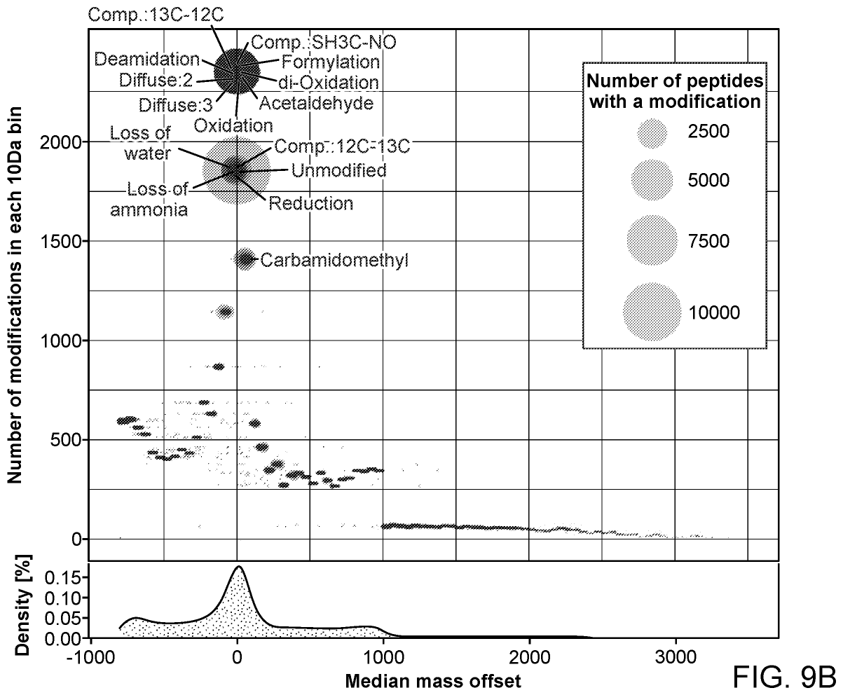

To examine if abundance differences between healthy and early NSCLC subjects were detected, protein groups and peptides that are differentially abundant (DA) were searched. First, to reduce potential noise introduced by rare peptides, protein groups were filtered to those present in at least 50% of subjects from either heathy or early NSCLC, retaining 10,280 peptides and 1,565 protein groups across 141 subjects (FIG. 1E). Next, as each protein group may have been detected by more than one nanoparticle (NP), MaxLFQ is used to quantify a single abundance (hereto referred to as collapsed abundances) between healthy and early NSCLC subjects. Differential protein group abundances were observed across 243 significantly regulated protein groups (adjusted p<0.05; Wilcoxon Test) (FIG. 1F and FIG. 1G). To investigate NPs capacity to capture biological signal beyond abundance levels (e.g., proteoform information, or NP-specific protein complexes), each NP: protein group feature pair was treated as a separate observation comparing healthy and early NSCLC subjects. Here, 877 NP: protein group feature pairs were identified (FIG. 2F), corresponding to a 3.6-fold increase from examining differences at the aggregated level alone. This highlights the capacity of NPs to interrogate the proteome: the signal they capture can be more biologically relevant than that captured by DA analysis without using NPs. Finally, differential abundance analysis was performed using peptide abundances across all NPs (i.e., not collapsed abundances) between healthy and early NSCLC subjects and identified 5,181 DA peptides (FIG. 1F and FIG. 1H), corresponding to a 6.5-fold increase from examining differences at the protein level. For a subset of samples, DDA analysis was performed in combination with a dependent peptide mapping to identify modified version of peptides. Across 29 samples, evidence of more than 30K modified peptides were found, that in a larger cohort may provide additional biological insights (FIGS. 9A-9B). These results indicate that by leveraging both NP specific features patterns and peptide information, the number of observable differences can be increased, which may lead to a better classification of phenotypes and additional mechanistic insights. Moreover, the rich fragmentation data acquired with an unbiased mass spectrometry readout can provide tens of thousands additional features including various types of modified peptides that can be interrogated for biological differences and to map proteoform information.

To test the utility of NP and peptide-level interrogation of complex biological samples, the top 10 most DA protein and peptides were examined. Among the top DA peptides, peptides mapping to ITIH2, ANTXR2, and ANTXR1 were observed, which are downregulated in early NSCLC plasma samples. Downregulation of ITIH2 expression can be been seen in 70% of breast cancers, 71% of lung cancers, and 70% of renal tumors. ANTXR2/CMG2 can inhibit breast cancer cell growth and can be inversely correlated with disease progression and prognosis. ANTXR1 can reduce tumor growth in vivo by targeting cancer stem cells in conjunction with LeTx. In agreement with results of other studies, the analysis of NSCLC plasma samples show upregulation of well-defined pro-inflammatory and cancer biomarkers such as CRP, S100A9, and S100A8. Together, the observation of known hallmark cancer and inflammatory biomarkers indicates PROTEOGRAPH™-derived proteomic data captures biological differences, which may include novel biomarkers. Overall, this increased number of observed significant differences between proteins, proteins across NPs, and peptides across NPs, verified by the presence of established cancer biomarkers, indicates that the concepts disclosed herein can be used to provide increased biological insight, by identifying proteoforms with increased resolution of proteomic data.

### Identification of Four NSCLC-Associated Proteoforms Using Peptide-Level Discordant Peptide Search

This example shows that DA peptides can be used to resolve proteoforms with improved detail. DA peptides were extracted, and from those, protein with at least one peptide over-expressed in healthy subjects and at least one peptide over-expressed in early NSCLC subjects were retained (FIG. 2A). Then, by mapping the DA peptides to genomic space, potential exon usage and proteoforms were inferred. The discordant peptide intensity analysis was performed to identify four proteins that were potentially captured, the four proteins having multiple protein isoforms with significant differential behavior in early NSCLC when compared to healthy controls: BMP1, C4A, CIR, and LDHB (FIG. 2B). The Open Target Score database was analyzed, where the database is an association score of established and potential drug targets with diseases using integrated genome-wide data from a broad range of data sources, to assess the association of the four proteins with lung carcinoma targets. Modest to low scores were found for the four proteins (FIG. 2B), suggesting a mix of novel and known lung cancer-relevant proteins. The proteins are established in plasma and range from highly abundant (C4A, CIR, LDHB) to moderately abundant (BMP1) (FIG. 2C). BMP1, the least abundant of the four proteins, is not identified in depleted plasma, indicating this approach enabled the identification and discovery of a protein isoform previously unknown. The results indicate that, using a MS-based peptide discordant intensity search, proteoforms are identified with possible relevance to NSCLC. Similarly, proteoforms may be identified that have relevance to other diseases.

To interrogate other potential isoforms of these proteoforms, differences in abundances were examined between healthy and early NSCLC subjects for each of the four at the collapsed protein level, NP: protein level, and peptide level. First examining BMP1, at the collapsed protein (FIG. 2D) and NP: protein (FIG. 2E) level, significant differences in BMP1 abundance were not observed. Without being bound to a particular theory, the lack of significant difference may be attributed to averaging of peptide abundances occurring at the protein-level. However, at the peptide-level (FIG. 2F), there are three significantly differential peptides: 1) peptide 1, which is significantly upregulated in early NSCLC subjects (adjusted p=5.29×10⁻⁴; Wilcoxon Test); 2) peptide 3, which is significantly upregulated in healthy subjects (adjusted p=1.21×10⁻²; Wilcoxon Test); and 3) peptide 7, which is significantly upregulated in healthy subjects (adjusted p=4.99×10⁻²; Wilcoxon Test). A trend is observed in the direction of abundance differences, where the first two peptides are upregulated in early NSCLC subjects and the last five peptides are upregulated in healthy subjects (FIG. 2F). To assess whether these two groups of peptides belong to different proteoforms, their abundance similarities were further compared across the 141 subjects. It is expected that peptides that belong the same proteoform should have correlated abundances across individuals since they belong to the same molecular entity while peptides belong to different proteoforms should have non-correlated abundances across a cohort of individuals. Thus, pairwise Pearson correlation and hierarchical clustering analysis were performed, which showed two distinct clusters driven by a high degree of correlation in peptide 1 and 2 (cluster 1) and peptides 3-7 (cluster 2) (FIG. 2G). The peptides were mapped to their genomic sequence, including four protein coding isoform transcripts (ENST00000397814, ENST00000354870, ENST00000306349, and ENST00000306385), and they were ordered according to exon order (FIG. 2H). Two distinct segments of corresponding direction of BMP1 peptide differential abundance were observed. Specifically, peptides 1 and 2 were both upregulated in early NSCLC subjects (segment 1) and peptide 3-7 were all upregulated in healthy subjects (segment 2) (FIG. 2F). Peptides mapping segment 1 exclusively map to exons present in the short isoform (ENST00000397814), whereas peptides mapping to segment 2 exclusively map to exons present in the three longer isoforms (ENST00000354870, ENST00000306349, and ENST00000306385) (FIG. 2H). Since segment 1 peptides, corresponding to the short BMP1 isoform, are upregulated in early NSCLC subjects and segment 2 peptides, corresponding to the longer BMP1 isoforms are upregulated in healthy subjects, these results may suggest distinct consequences of the long and short BMP1 isoforms. As BMP1 can play a dual role in cancer, acting as both a suppressor and activator, these results may reveal a role of BMP1 isoform abundance in cancer.

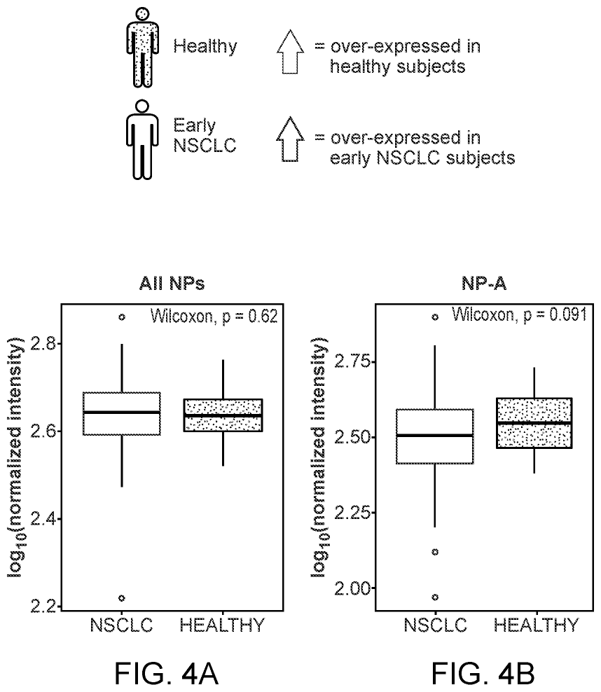

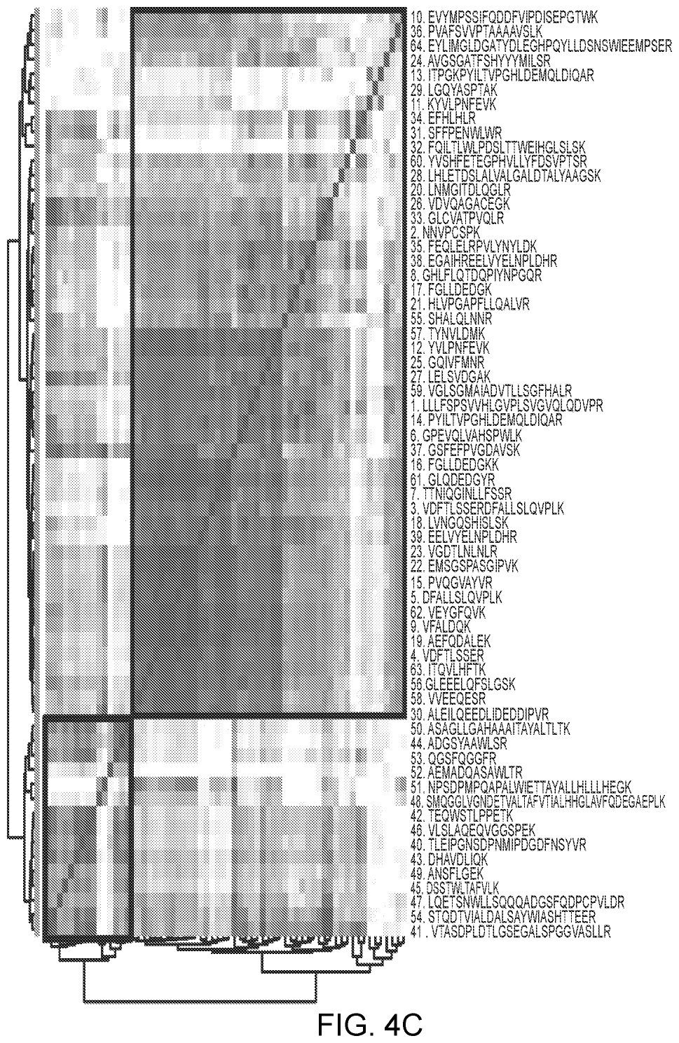

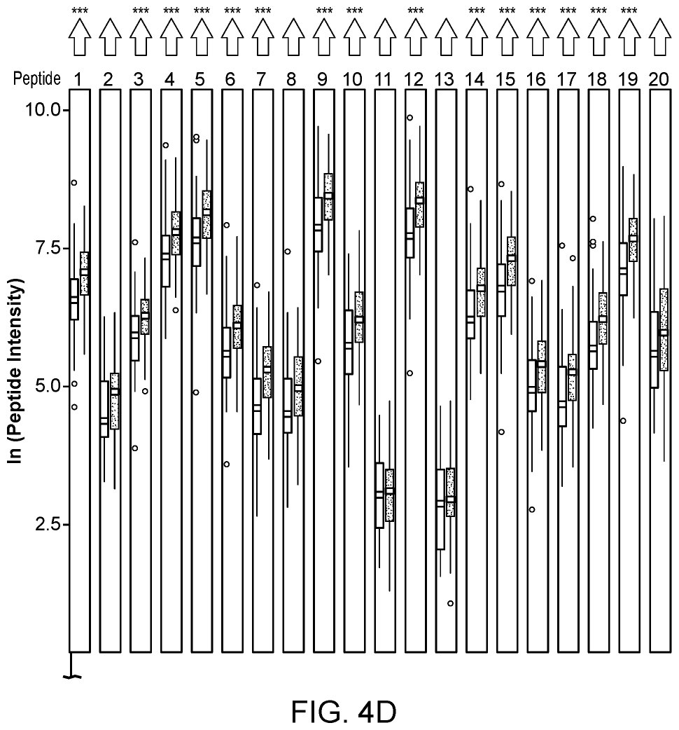

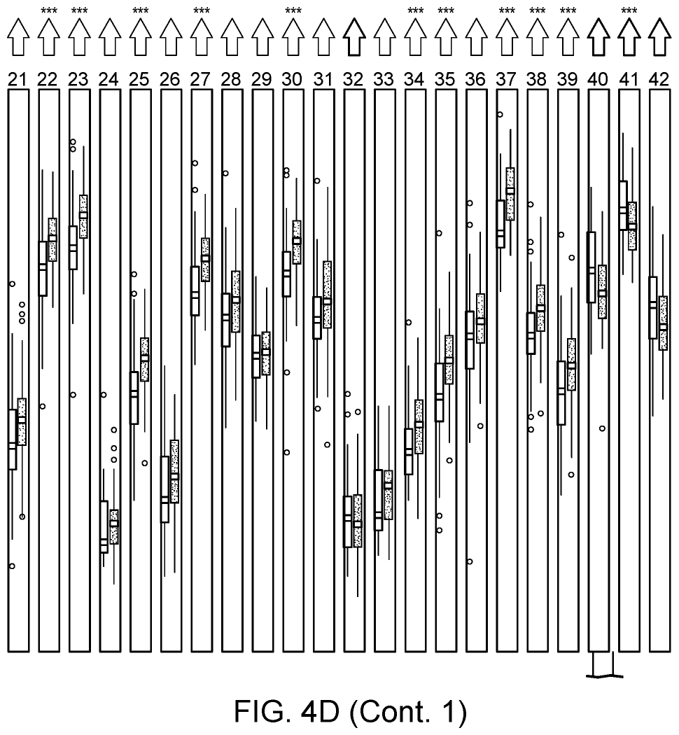

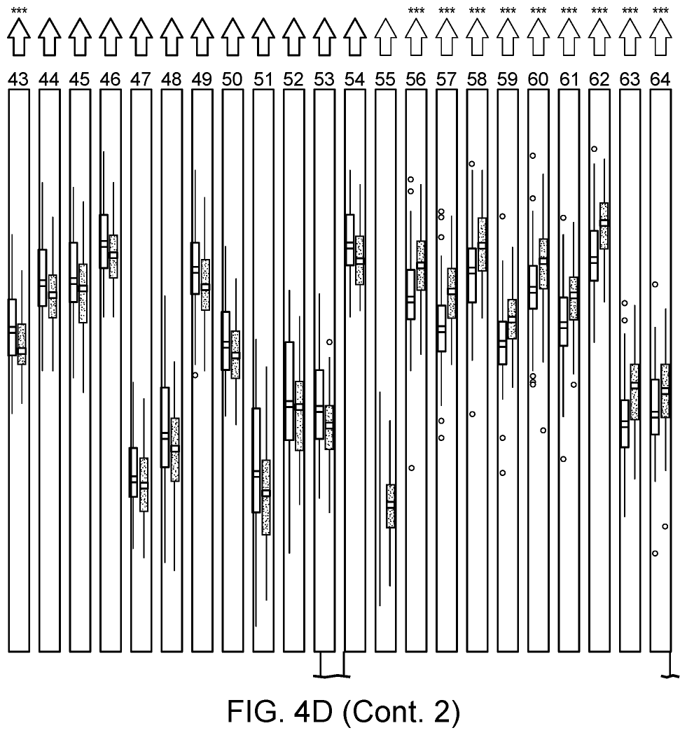

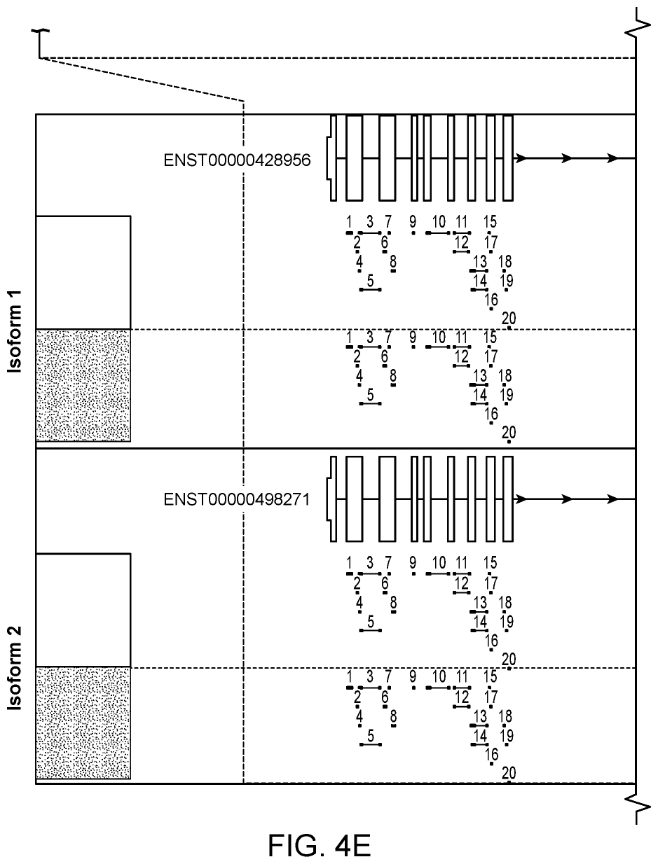

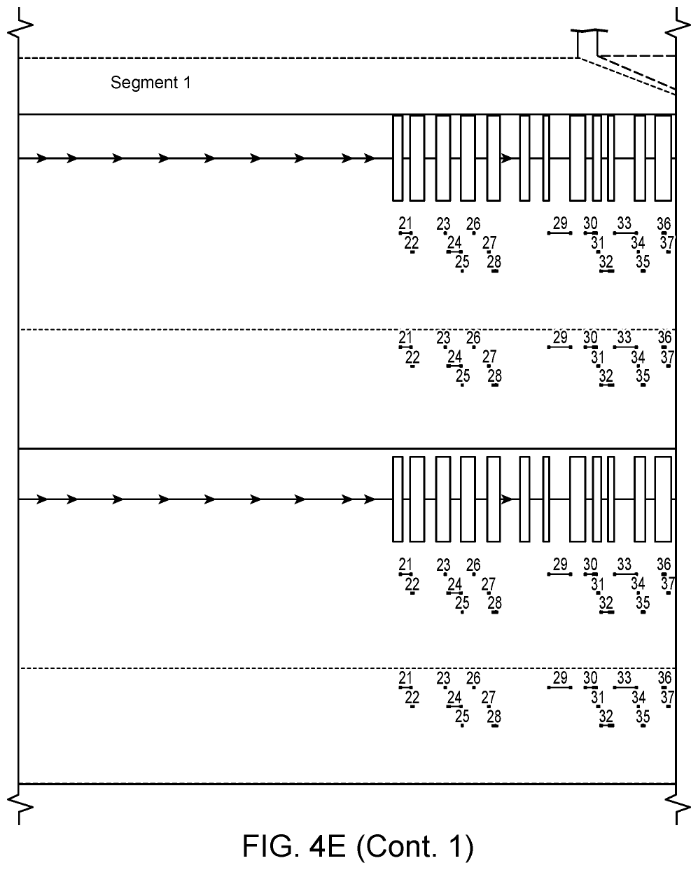

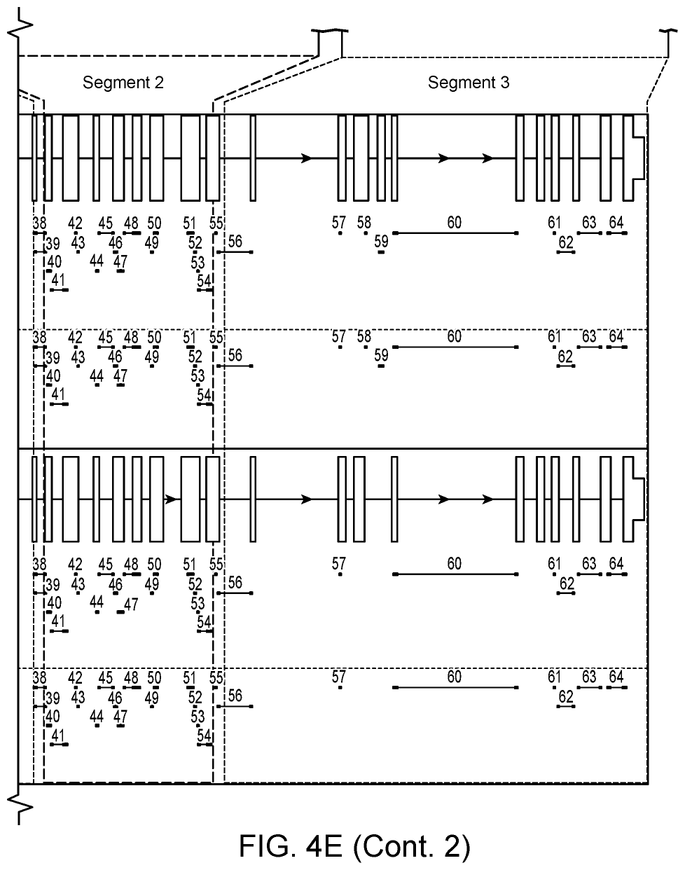

To interrogate the potential different isoforms, C4A was next examined. At the collapsed protein (FIG. 4A) and NP: protein (FIG. 4B) level, as for BMP1, the difference in C4A abundance is not statistically significant, however at the peptide level there are many significantly differentially expressed peptides (FIG. 4D). Like BMP1, this further supports protein abundance comparisons mask differences that occur at the peptide level. Pairwise Pearson correlation and hierarchical clustering analysis showed two distinct clusters driven by peptide abundance correlation in peptides 40-54 (cluster 1) and peptides 1-39 and 55-64 (cluster 2) (FIG. 4C). Mapping the peptides to the two known protein coding isoform transcripts (ENST00000428956 and ENST00000498271) and ordering them according to exon order (FIG. 4E), three distinct segments of corresponding direction of C4A peptide differential expression abundance were observed. Specifically, peptides 1-39 were upregulated in healthy subjects (segment 1), peptides 40-54 were upregulated in early NSCLC subjects (segment 2), and peptides 55-64 were upregulated in healthy subjects (segment 3) (FIG. 4D). Interestingly, segments 1 and 3 correspond to cluster 2 and segment 2 corresponds to cluster 1, indicating discordant expression of NSCLC-associated exons in segment 2. In contrast to results from analysis BMP1, there was no obvious association with the two known C4A protein coding isoform transcripts. However, the discordant peptides of segment 2 suggest the presence of an unknown isoform or a smaller byproduct from C4A or other members of the C4 complex.

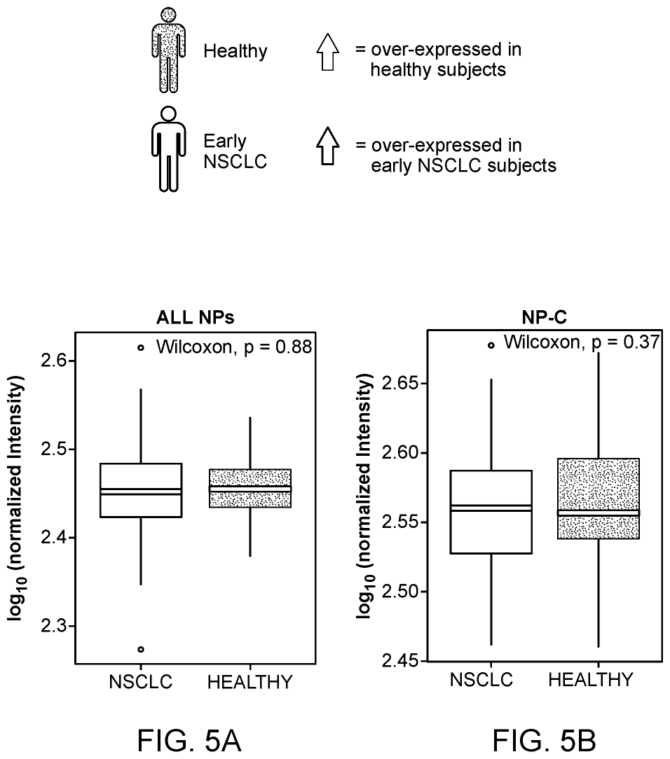

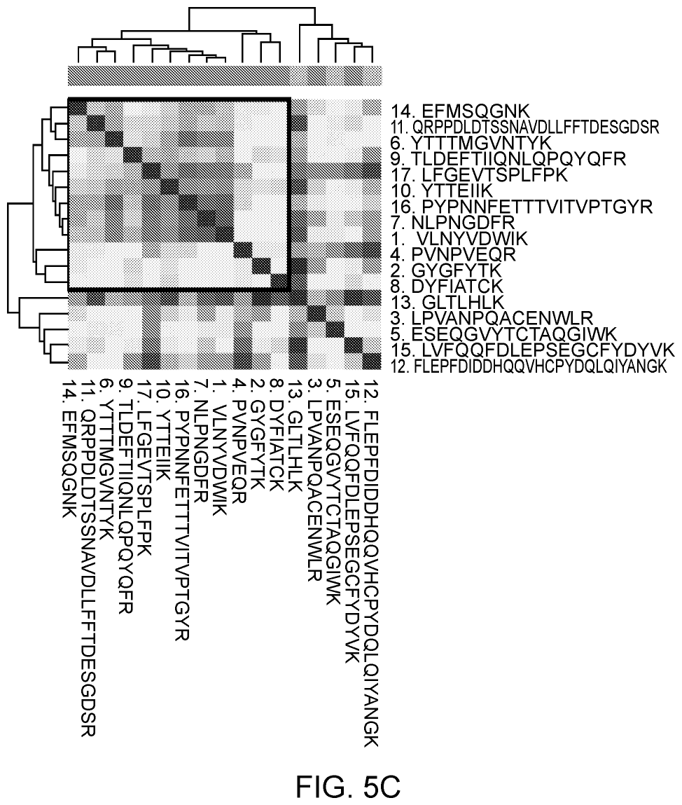

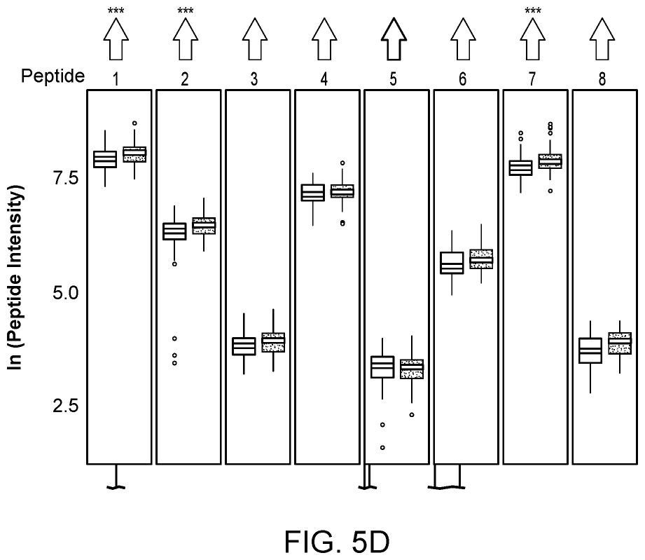

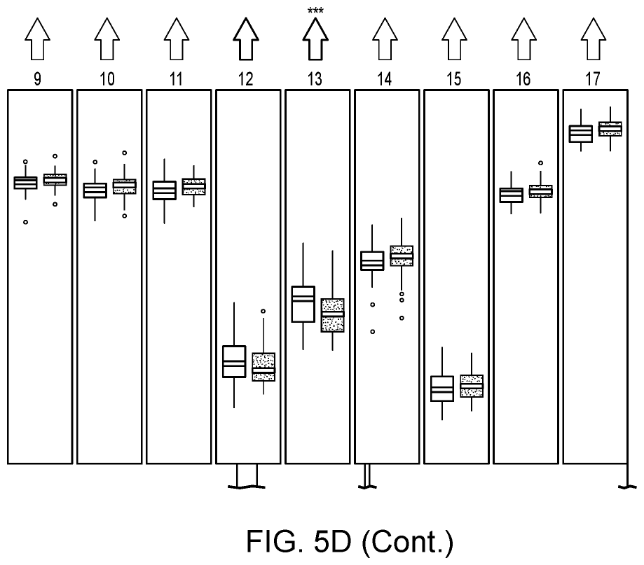

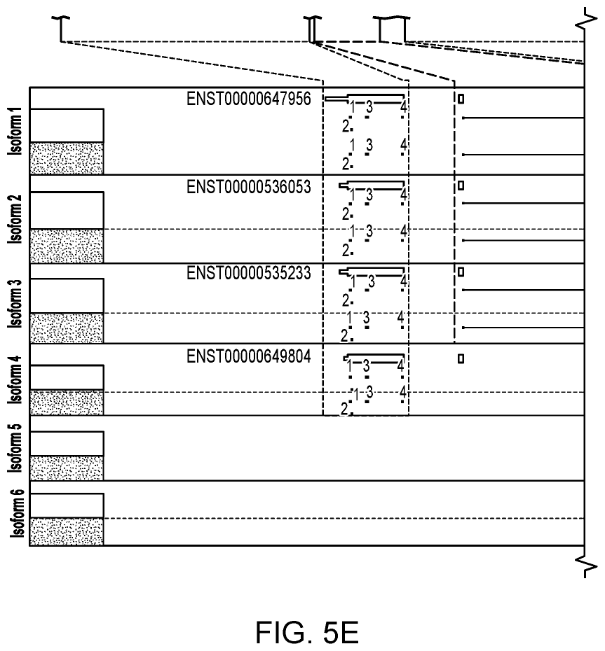

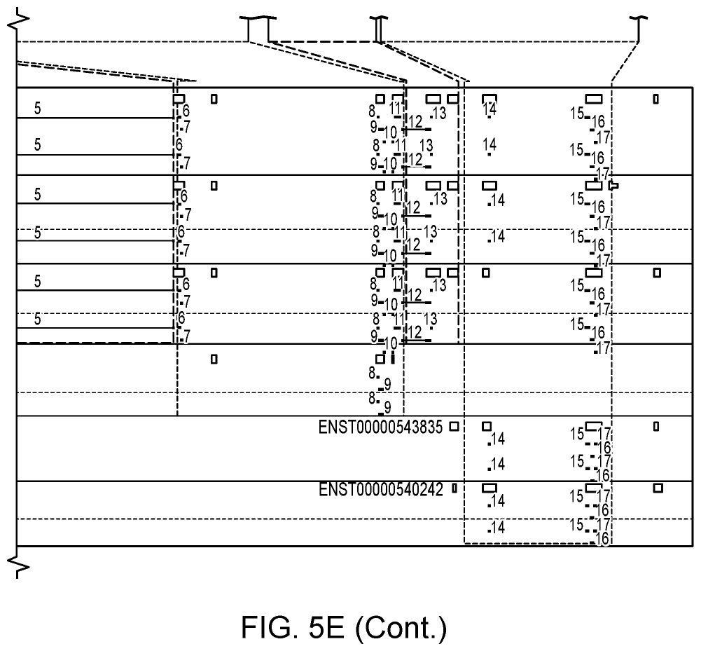

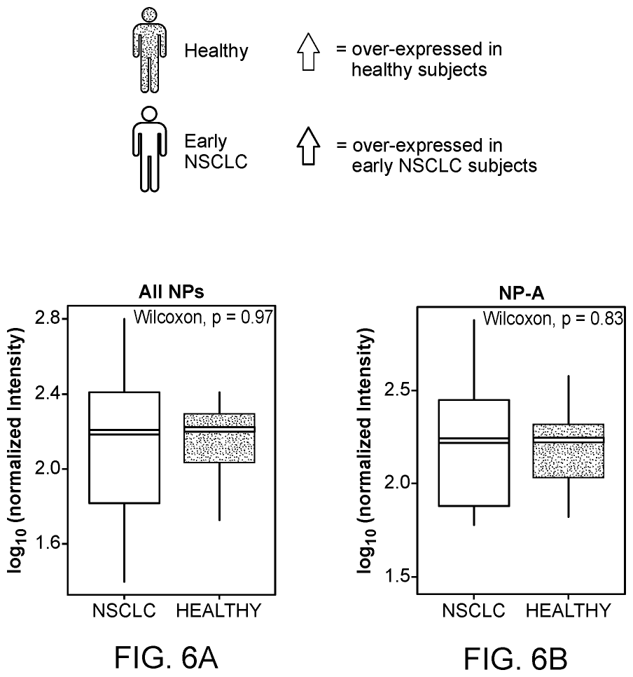

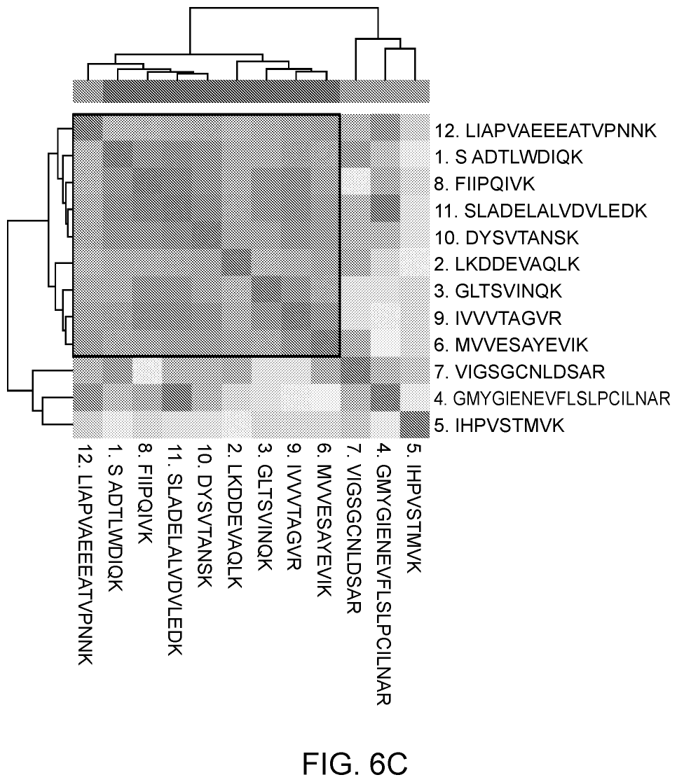

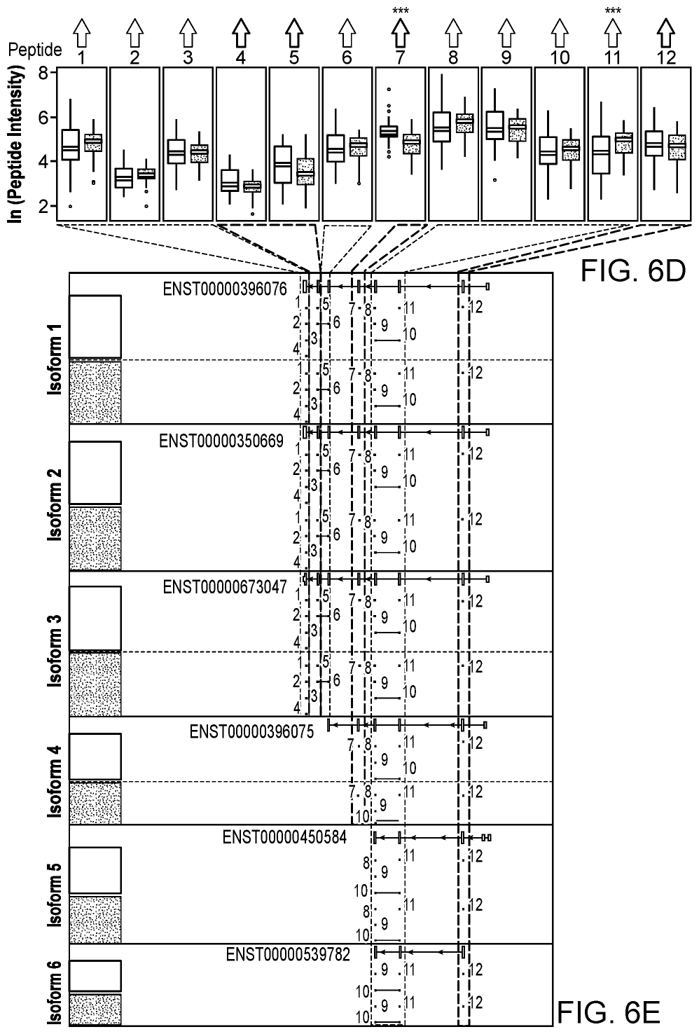

To interrogate the potential different isoforms, CIR and LDHB were next examined. At the collapsed protein (FIG. 5A, FIG. 6A) and NP: protein (FIG. 5B and FIG. 6B) level, the difference in CIR and LDHB abundance, respectively, was not statistically significant. However, at the peptide level differentially expressed peptides were observed (FIG. 5D and FIG. 6D), indicating protein abundance comparisons mask differences that occur at the peptide level. Pairwise Pearson correlation and hierarchical clustering analysis showed one distinct cluster in each protein. For CIR, this consisted of moderately correlated peptides 1, 2, 4, 6-11, 14, 16, and 17 (cluster 1) and a weak correlation between peptides 3, 5, 12, 13, and 15 (FIG. 5C). For LDHB, this consisted of highly correlated peptides 1-3, 6, and 8-11 (cluster 1) and a weak correlation between peptides 4, 5, and 7 (FIG. 6C). The peptides were mapped to the known protein coding isoform transcripts (CIR: ENST00000647956, ENST00000536053, ENST00000535233, ENST00000649804, ENST00000543835, and ENST00000540242; LDHB: ENST00000647956, ENST00000536053, ENST00000535233, ENST00000649804, ENST00000543835, and ENST00000540242) and ordered them according to exon order (FIG. 5E and FIG. 6E). There was no clear pattern in healthy or NSCLC subject peptide upregulation corresponding to any of the known isoforms for either protein. However, upregulation in CIR peptides 14-17 in healthy subjects were observed, corresponding to the two short isoforms (isoforms 5 and 6; FIG. 5E). Further examination of CIR and LDHB protein isoforms is needed to further explain the discordance in peptide abundances. Together, these results indicate discordant peptide abundance can be utilized to identify some disease-relevant protein isoforms, as was observed in the case of BMP1 and C4A. However, this approach cannot explain all discordance, as was observed in the case of CIR and LDHB.

Proteoforms Arising from Genetic Variation can be Identified Using a Proteogenomic Approach

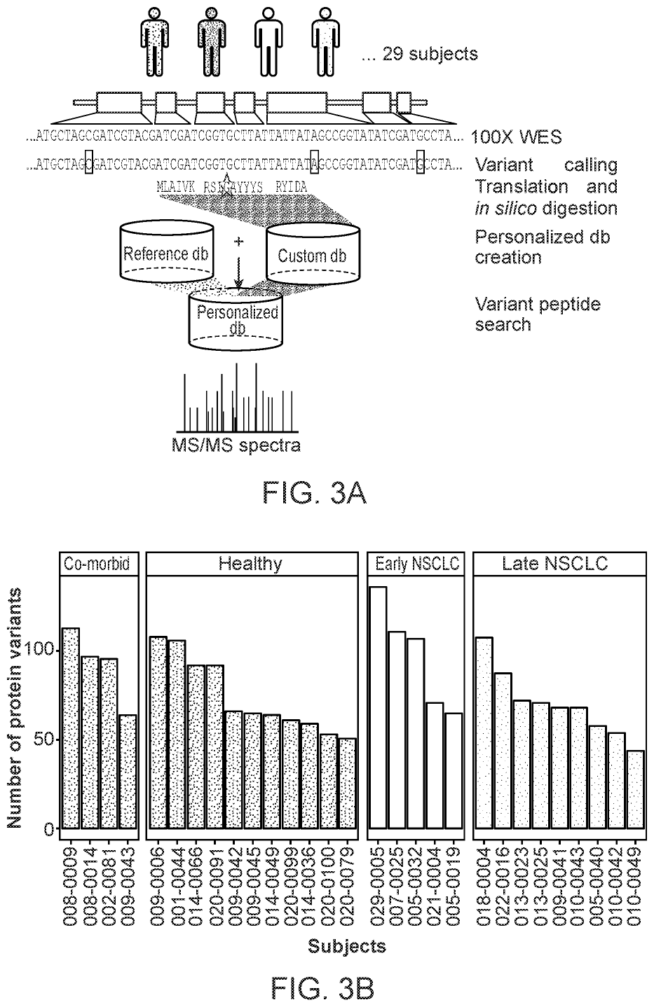

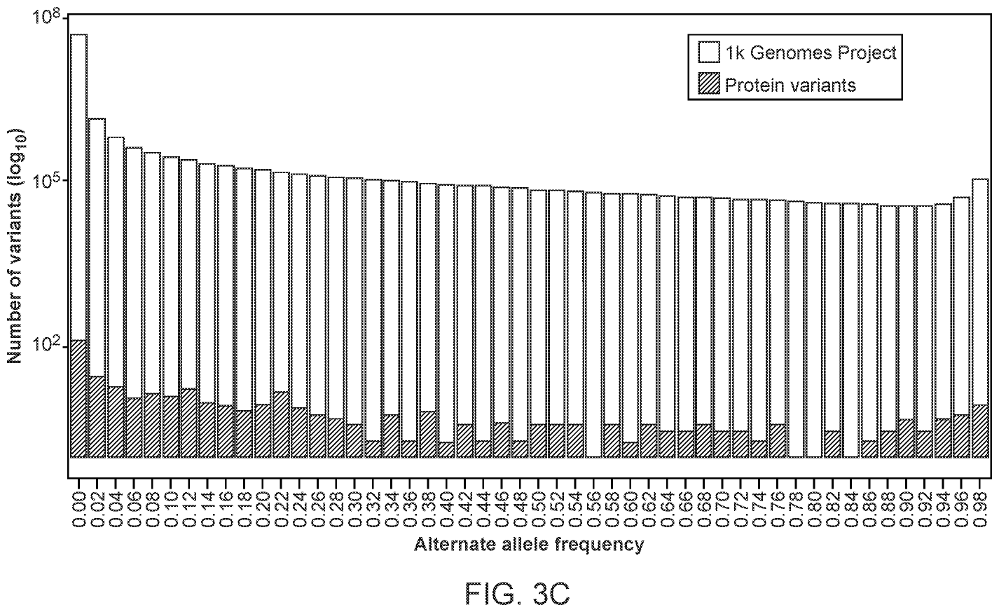

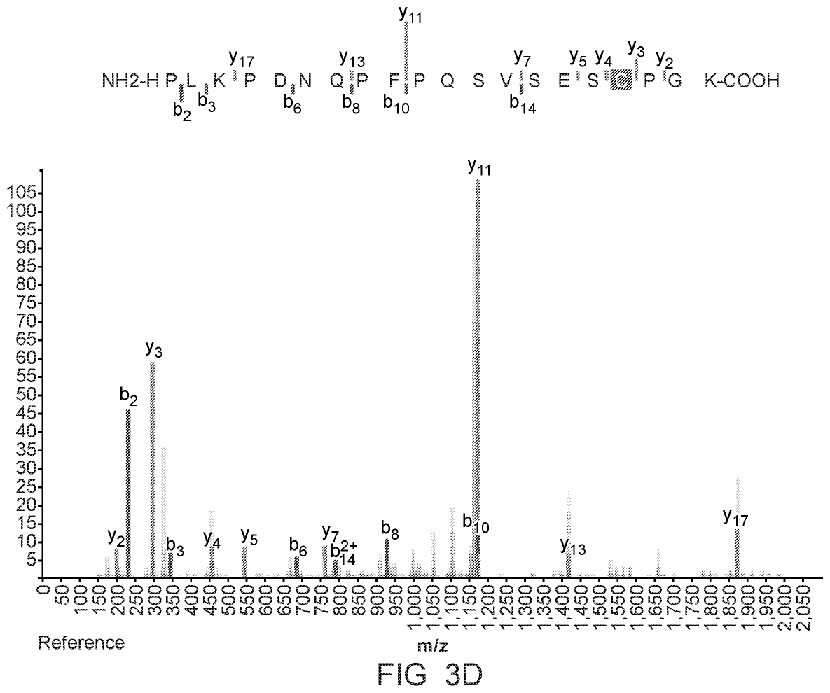

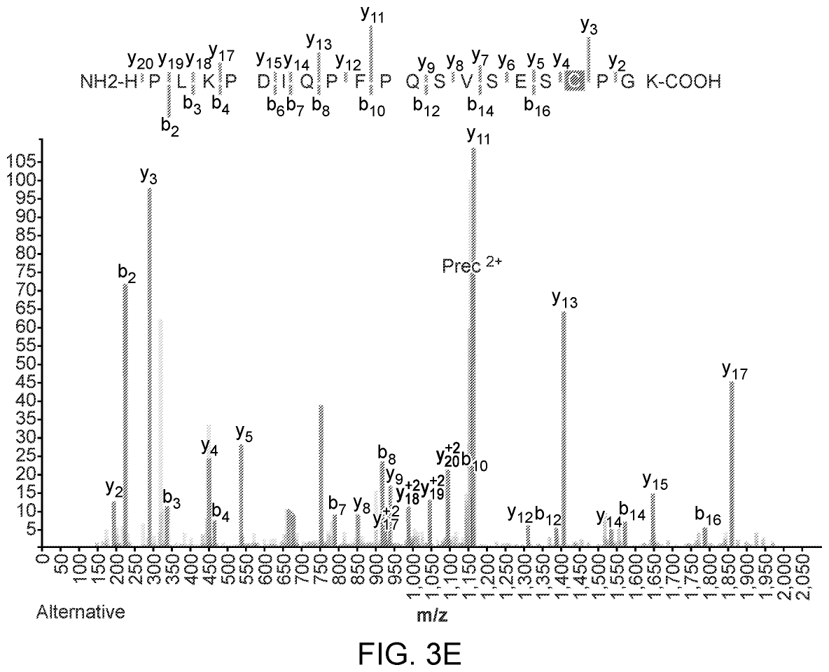

DDA data from 29 subjects were obtained (11 healthy subjects, 5 early NSCLC, 9 late NSCLC subjects, and 4 comorbid subjects), for which WES data was generated. This data was utilized to perform custom proteogenomic searches and to identify protein variants (FIG. 3A). Optionally, WES-derived variant information may be incorporated to generate personalized databases. Specifically, for each subject, single nucleotide variants (SNVs) were identified that result in single amino acid variants (SAAVs), and the SNVs were used to generate custom peptide sequences that could exist in each individual subject's proteome (i.e., personalized peptide sequences). All these variant peptides were added to the canonical proteins sequences from the reference human proteome and were used for MS/MS peptide identification. Protein variants were searched across all 29 subjects, mapping 422 protein variants with an average of 79.59±23.57 protein variants per subject (FIG. 3B). Optionally, the protein variants may be searched within personalized databases. The alternative allele frequencies of identified protein variants were examined. The distribution that follows what is established for population-scale studies was found, including the observation of rare alleles (FIG. 3C). For example, it was detected a peptide variant harboring a SAAV (H→R) resulting from an established genetic variant related to lung cancer, rs1229984, in one early NSCLC individual. This genetic variant changes a histidine to arginine, leading to a detectable short alternative variant peptide. Interestingly, the reference peptide was not observed, possibly due to the long segment of the protein without an arginine or lysine site for trypsinization. Additionally, peptides were observed in which both the reference and alternative alleles from heterozygous variants were identified. For example, in an early NSCLC subject, 021-004, a SAAV (N→I) was detected, for which both the reference (SEQ ID NO: 1) and alternative peptide (SEQ ID NO: 2) mapping to HRG (FIGS. 3D-3E) were identified, indicating that peptide search results from standard searches are recapitulated in addition to capturing novel and personalized peptides. Together, these results demonstrate the potential of peptide-centric proteogenomic and NP-based workflow for gaining new biological insights into NSCLC and other diseases.

### Discussion

Existing technologies, including an unbiased NP-based methodology upstream LC-MS/MS-based workflows and targeted methodologies, have enabled protein-centric analyses that have revealed important insights into human disease. While protein-centric analyses have made substantial strides in the understanding of biology, protein-level studies may conceal biologically critical features, like proteoforms arising from alternative splicing (protein isoform), allelic variation (protein variants), or post-translational modifications, which can provide mechanistic insights underlying complex traits and disease. Importantly, unbiased LC-MS/MS-based proteomic data can enable peptide-centric analyses that may reveal new discoveries surrounding proteoforms. A rationale for this study is that peptide-level information can be derived from LC-MS/MS data and can enable proteoform identification using discordant peptide abundance and proteogenomic search analyses. In some cases, protein inference engines can use peptide-level data to detect the presence or absence of peptides to identify protein isoforms. However, here it is shown the utility of incorporating quantitative profiles of peptides mapping to known isoforms in potentially increasing the sensitivity of proteoform detection. Thus, it was shown that LC-MS/MS plasma proteomic data can be analyzed at the peptide-level and using quantitative profiles to infer protein isoforms to yield deeper insights into putative disease mechanisms. Here, it is demonstrated that a peptide-centric analysis of NP-based methodologies can indicate both established and novel disease-relevant proteoforms.

Peptide analysis was performed using DIA data derived from healthy and early NSCLC subjects by conducting a discordant peptide intensity search to identify protein isoforms. Four proteins with putative isoforms were identified, including BMP1, C4A, CIR, and LDHB. None of these proteins showed a statistically significant difference in abundance at the protein-level. For BMP1 and CIR, using peptide abundance as a proxy for functionally relevant protein, potential NSCLC-related isoforms were identified. BMP1 is known to act as both a suppressor and activator, a function that can be linked to differential abundance of two isoforms (long and short). Additionally, C4A showed distinct peptide abundance discordance in one segment of the protein, which did not correspond to any known protein coding isoforms, suggesting peptide-centric proteoform identification may result in novel disease-associated isoforms.

The method used to search for protein isoforms through discordant peptide intensity is easily interpretable. Similar approaches such as COPF [34155216] and PeCorA use quantitative disagreements between peptides mapped to the same protein or peptide correlation within the same protein to detect protein isoforms and suggest proteoforms. However, as shown herein, 2 of the 4 isoform candidates (CIR and LDHB) met the discordant peptide intensity criteria but failed to be readily explained by known isoforms or biological conjecture. The process was mapping the peptides back to the genomic sequence and isoform transcripts. Manual validation (e.g., isoform specific enrichment with isoform specific antibodies) can confirm the presence of novel isoforms. Manual validation was achievable for the four candidates from the isoform detection process, however, other processes such COPF and PeCorA may yield more candidates. A robust and automated evaluation method of true isoforms is disclosed herein.

In addition, subject-specific genotype data derived from WES can reveal subject-specific protein variants. Across 29 subjects, 422 protein variants were identified, for which peptides were observed harboring SAAVs not present in standard peptide sequence search databases. Among these protein variants, a protein harboring a genetic variant was detected, rs1229984, with significant association with lung cancer, as well as cases where both the reference and alternative alleles were observed. Low frequency alleles or genetic variations that significantly alter the physicochemical properties of a protein's surface may sometimes be inaccessible for targeted affinity-based proteome screening tools (e.g., aptamer and antibody-based methodologies), demonstrating the unique synergy of NPs-based proteomics workflows coupled to unbiased LC-MS/MS readouts as disclosed herein.

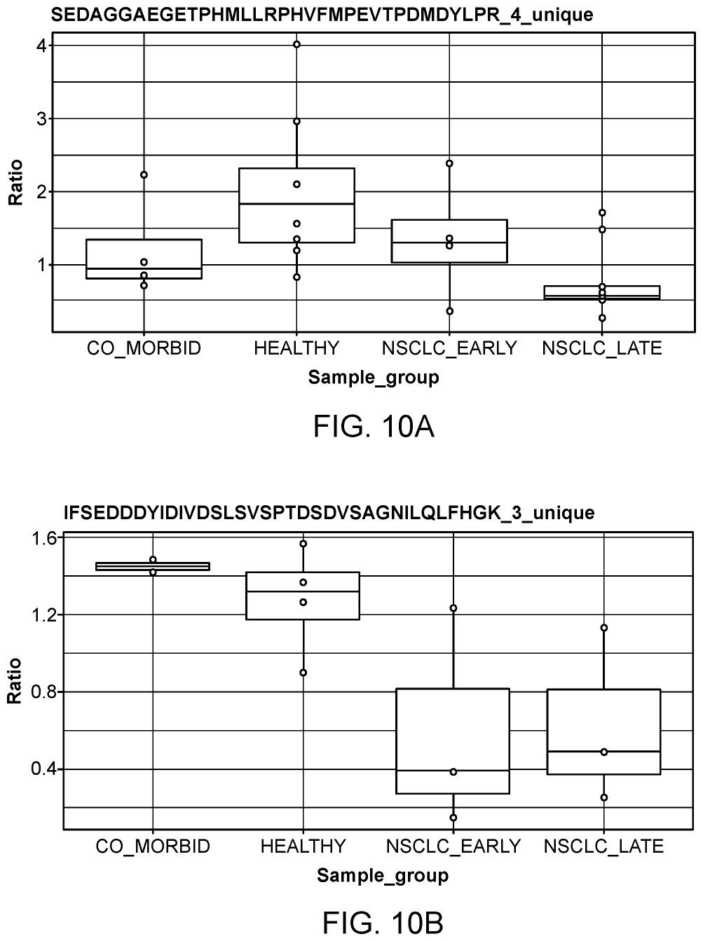

Since the study shows the utility of using NP-based methodology upstream LC-MS/MS-based workflows to identify proteoforms, it is possible that expanding the sample size and diversity in sample type may yield further insights into disease-associated proteoforms. Similarly, it is also possible that applying the methods and systems disclosed herein to other sample types may lead to discovery of previously unknown proteoforms. LC-MS/MS enables quantifying and identifying tens of thousands of peptides with post-translational modifications precisely defined by their intact mass and fragmentation pattern. These peptides can further provide rich information about proteoforms that are presented in the samples. To illustrate this, exploratory analysis was performed to look for phospho-peptides as well as peptides with unspecified PTMs in NSCLC samples (FIGS. 9A-9B and FIGS. 10A-10B). Several search strategies were considered. For instance, dependent peptide searches look for an overlap of fragmentation patterns between known peptides and unidentified features and in conjunction with a delta mass to indicate post-translational modifications. In contrast to analyte specific assays, such features can be subjected to further investigations or directly serve as distinguishing patterns comparing e.g., disease stages.

The identification of proteoforms (protein isoforms and protein variants) highlights important considerations for approaches characterizing the impact of genetic variation on molecular phenotypes, like protein abundance, by conducting protein quantitative trait analyses (pQTLs). Some pQTL analyses, using large cohorts, are performed at the protein level and thus may miss or misattribute peptide level, proteoform effects. Furthermore, these studies utilize aptamer and antibody-based methodologies that can lead to false discoveries and uncertain identification error rates because of conceptual limitations (e.g., the presence of a non-synonymous SNP inducing an amino acid change that disrupts the binding of the aptamer or antibody). Disclosed herein is a large-scale LC-MS/MS-based peptide identification, proteoform identification and pQTL methodology across multiple biofluids and tissues.

NSCLC sample Collection

As part of an IRB-approved study, plasma samples were collected from subjects from 24 collection sites diagnosed with NSCLC at stage 1, 2, 3, and 4 post-diagnoses but before treatment, as well as samples from healthy and pulmonary comorbid subjects as controls. Diagnosis of NSCLC was based on CT-guided fine-needle aspirant biopsy. Respective collection site has IRB (Supplementary Data 1 in J. E. Blume et al. Rapid, deep and precise profiling of the plasma proteome with multi-nanoparticle protein corona. Nat. Comm. 11, 2020, which is incorporated herein by reference in its entirety) approved protocols (Supplementary Note 1 in J. E. Blume et al. Rapid, deep and precise profiling of the plasma proteome with multi-nanoparticle protein corona. Nat. Comm. 11, 2020, which is incorporated herein by reference in its entirety) and written informed consent from all subjects for obtaining blood samples from NSCLC subjects. For the healthy and pulmonary comorbid controls, subjects were enrolled based on call-backs at collection sites. Healthy controls did not have current diagnosis of any form of cancer or any pulmonary co-morbidities such as COPD or emphysema. All subjects were not necessarily fasted at the time of collection. For the plasma samples, they were collected in EDTA tubes, centrifuged, aspired, frozen, and stored at −70° C. within one hour of collection; Subsequent shipments of samples were on dry ice. Prior to PROTEOGRAPH™ processing, plasma samples were thawed at 4° C., aliquoted, and refrozen. Wilcoxon and Fisher tests on age and gender, respectively, did not show significant differences between control and NSCLC subjects.

### DIA Data Generation

Peptides were reconstituted in a solution of 0.1% FA and 3% ACN spiked with 5 fmol/uL PepCalMix from SCIEX (Framingham, MA) for the SWATH-DIA analysis. A constant injection mass of 5 μg of peptides per 10 uL MS volume was targeted, but when lesser yield was observed, the maximum amount was injected. The mass spectrometer was operated in SWATH mode using 100 variable windows across the 400-1250 m/z range. A trap-and-elute configuration was used for each sample using an Eksigent nano-LC system coupled with a SCIEX Triple TOF 6600+ mass spectrometer equipped with OptiFlow source. Peptides were loaded on a trap column and separated on an Eksigent ChromXP analytical column (150 mm×15 cm, C18, 3 mm, 120 Å) at a flow rate of 5 uL/min using a gradient of 3-32% solvent B (0.1% FA, 100% ACN) over 20 min, resulting in a 33 min total run time.

### Peptide-Spectral Library Generation

To build a peptide-spectral library four plasma pools were used. The four plasma pool were created from the patients in the lung cancer, depleted using a MARS-14 column (Agilent, Santa Clara, CA) and the Agilent 1260 Infinity II HPLC system, and analyzed by the PROTEOGRAPH™ using the panel of 10 NPs. Data-dependent mode was used on the Ultimate 3000 RSLCnano system coupled with Orbitrap Fusion Lumos using a gradient of 5-35% over 109 min, for a total run time of 125 min. To expand the spectral library, a separate pooled plasma consisting of 157 healthy and lung cancer subjects were also used, depleted using the MARS-14 column and fractionated into nine concatenated fractions with a high-pH fractionation method (XBridge BEH C18 column, Waters), and analyzed using the 10 NPs panel. Same DDA mode and parameters were used as the NSCLC samples. Finally, all DDA generated spectra were searched against human UniProt database using the Pulsar search engine in Spectronaut (Biognosys, Switzerland), and the final library was generated with a 1% FDR cutoff at the peptide and protein group level.

### DIA Data Processing

Spectronaut was used to process the SWATH-DIA data at default settings (version 13.8.190930.43655), with a Q-value cutoff at precursor and protein group levels of 0.01 were used (Supplementary Data 2 in J. E. Blume et al. Rapid, deep and precise profiling of the plasma proteome with multi-nanoparticle protein corona. Nat. Comm. 11, 2020, which is incorporated herein by reference in its entirety)) Per an ongoing, IRB-approved observational sample collection protocol, samples for 288 subjects as part of an NSCLC study were assayed across a 10-nanoparticle panel. Subjects diagnosed with NSCLC stage 1, 2, and 3 were labeled as early NSCLC. Subjects with NSCLC stage 4 were labeled as Late NSCLC. In addition, healthy and pulmonary comorbid control arms were used. Subjects diagnosed with NSCLC but with Unknown stage were removed from analysis; subjects who did not have peptides detected in all nanoparticles in the 10-NP panels were also removed. Summary statistics of protein group counts and peptide counts per protein group were calculated at this point.

Next, protein groups were filtered to those present in at least 50% of subjects from either heathy or early cases, leaving a total of 141 subjects (80 control and 61 early NSCLC). Peptide intensities were median normalized and natural logged.

### Identification of Protein Isoforms

From the 1,565 protein groups present after filtering, peptides were searched that had differential abundance between controls and cancer (p<0.05; Benjamini-Hochberg corrected). Discordant pairs can be defined as peptides from the same protein group where at least one peptide was identified with significantly higher and another peptide was identified with significantly lower plasma abundance in healthy controls vs. early NSCLC.

### Protein Quantification Across Multiple Samples

Within each nanoparticle, standard MaxLFQ was used to quantify abundance at the protein group level. For each peptide, the intensity ratios between every pair of samples were first computed. The pairwise protein group ratio is then defined as the median of the peptide ratios from all peptides map to the same protein group. With all the pairwise protein ratios between any two samples, a least-squares analysis was performed to reconstruct the abundance profile optimally satisfying all the protein ratios. Then the whole profile is rescaled to the cumulative intensity across samples for the final protein abundance. A modified MaxLFQ was used to quantify abundance across samples and nanoparticles. For each protein group, all peptides' intensities belonging to a protein group from all samples and NP were employed to calculate peptide ratios and subsequent calculation steps resulting in abundance across all samples and NP.

### WES Data Generation and Processing

For 29 healthy, early NSCLC, late NSCLC, and comorbid subjects, DNA was extracted from buffy coats using QIAsymphony and eluted into 200 μl elution buffer. Sequencing libraries were then prepared using a TruSeq Exome Library Prep kit for each of the samples. Libraries were then sequenced on two lanes of NovaSeq S4 PE 101PE and received between 48M-88M reads and 9Gb-17Gb each. The bcl files were demultiplexed using BCL2FASTQ. DRAGEN Host Software Version 07.021.510.3.5.7 and Bio-IT Processor Version 0x18101306 were used to call variants using the reference genome hg38 and generate a vcf file.

### DDA Data Generation and Protein Variant Identification

Similar to methods in, custom protein database was generated from human hg38 genomic FASTA, BED file from UniProt that describes that gene structure and VCF file from whole exome sequencing. Reference allele was generated using the FASTA file for nucleotide sequence and the BED file for the gene model with information on the location of the exons and the frame at which to translate the codons into amino acid sequence. For the alternative alleles, instead of generating an entire protein sequence, tryptic peptides were generated that span each specific mutation described in VCF file. If multiple variants are observed within a peptide, all possible combinations of the mutations are generated as peptides. A custom sequence database was generated that contains the reference/canonical protein sequence and all the variant peptides from 29 individuals. MS/MS spectra from DDA data were searched against the custom protein database using the default Fragpipe pipeline (Fragpipe v15.0, Philosopher v.4.1.0 and MSFragger v3.4). For variant peptide identification, a 1% variant-peptide-level FDR was enforced using the target decoy approach. For phospho peptide identification, the default “phospho-labile” workflow from Fragpipe was used and results were filtered at 1% peptide level FDR.

### Dependent Peptide Analysis

The DDA runs for 29 healthy, early NSCLC, late NSCLC, and comorbid subjects were processed using MaxQuant (v2.0.3.0) enabling match between runs and dependent peptides identification at 1% FDR.

### Example 2: Blood Sample Collection Kit

This example describes a kit for collecting samples. A kit may comprise (i) Venipuncture equipment: 21 g butterfly needle, vacutainer holder, alcohol prep pad, tourniquet, 2×2 sterile gauze, and bandage; (ii) cryolabels; (iii) five blood tubes: two K2 EDTA tubes, one serum separator (SST), one PAXgene, and one Streck cell-free DNA BCT; eight 5.8 mL pipettes (2 mL volume with 500 uL graduation); three 15 mL conical Falcon tubes; thirty 2.0 mL cryovials; 9×9 cryobox; ice for transporting k2 EDTA tubes until centrifuge and between aliquot and freeze; instructions.

### Instructions

The instructions may comprise a list of the aforementioned components. The instructions may comprise shipping instructions. The instructions may comprise labeling instructions. The instructions may comprise sample numbering instructions. The sample numbering instructions may comprise at least one of: site number, subject ID, tube number, and aliquot.

The instructions may comprise a procedure using a K2 EDTA tube. The procedure may comprise one or more the steps of:
1. Immediately after tubes are full, slowly invert 8-10 times.
2. Centrifuge within 30 minutes of collection at 1100-1300 g for 15 minutes.
3. Immediately following centrifugation, place tubes on ice until samples are aliquoted.
4. Using a 5.8 mL pipette, remove the plasma from each k2EDTA tube using care to not disturb the buffy coat layer.
5. Transfer the plasma to a 15 mL conical Falcon tube; invert the Falcon tube 5 times to mix, then put the 15 mL Falcon tube on ice.
6. Using a second 5.8 mL transfer pipette, transfer 500 μL aliquots into labeled 2.0 mL cryovials.
6a. Draw up 1 mL into the transfer pipette (up to the bulb)
6b. Dispense 2,500 μL aliquots into 2 cryovials.
6c. Repeat, making as many 500 μL aliquots as possible; you will have about 8-10 aliquots.
6d. If left with less than 500 uL after aliquoting, fill the last cryovial tube with whatever volume is remaining.
6e. Keep aliquots on ice until they are transferred to freezer.
7. Using a third 5.8 mL transfer pipette, remove the buffy coat layer and transfer to a 2.0 mL cryovial.
8. Repeat the process for the second K2 EDTA tube.
9. Immediately following aliquoting, place the cryovial tubes in ice until transfer to the −20° C./−70° C./−80° C. freezer.
10. Within 1 hour of completion of centrifugation, transfer cryovial tubes to a 9×9 cardboard cryobox, and place cryobox in the −20° C./−70° C./−80° C. freezer.
11. Ke EDTA tubes may be discarded.

The instructions may comprise a procedure using a serum separator (SST). The procedure may comprise one or more the steps of:
1. Allow blood to clot for at least 30 min but not more than 1 hour at room temperature.
2. Centrifuge samples at 1100-1300 g for 20 minutes.
3. Using at 5.8 mL pipette, remove the serum from the SST tube and transfer to a 15 mL conical Falcon tube; invert the Falcon tube 5 times to mix.
4. Using a second 5.8 mL transfer pipette, transfer 500 μL aliquots to pre-labeled 2.0 mL labeled cryovials.
4a. Draw up 1 mL into the transfer pipette (up to the bulb)
4b. Dispense 2500 μL aliquots into 2 cryovials.
4c. Repeat, making as many 500 μL aliquots as possible; there will be about 8-10 aliquots.
4d. If left with less than 500 uL after aliquoting, fill the last cryovial tube with whatever volume is remaining.
5. Within 1 hour of completion of centrifugation, transfer cryovial tubes to a 9×9 cardboard cryobox, and place cryobox in the −20° C./−70° C./−80° C. freezer.
6. SST tube may be discarded.

The instructions may comprise a procedure using PAXgene. The procedure may comprise one or more the steps of:
1. Allow at least 10 seconds for a complete tube draw, and ensure that the blood has stopped flowing before stopping blood draw.
2. Immediately after collection, gently invert the tube 8-10 times.
3. Store upright at room temperature (18-25° C.) for 2-72 hours.
4. Transfer to −20° C./−70° C./−80° C. freezer; store in a wire (non-Styrofoam) rack.

The instructions may comprise a procedure using Cell-free DNA Streck BCT. The procedure may comprise one or more the steps of:
1. Fill tube completely.
2. Immediately after collection, gently invert the tube 8-10 times.
3. Store upright at room temperature until packaged for shipment; tubes must not be refrigerated or frozen.
4. Ship on the day of collection or, if collected late in the day, the next day.

The instructions may comprise a form for record keeping. The form may comprise one or more fields of:
1. Subject ID.
2. Date sample was collected.
3. Time sample was collected.
4a. Time centrifuge started for K2 EDTA tubes.
4b. Time centrifuge completed for K2 EDTA tubes.
4c. Time aliquots placed in −80° C. freezer for K2 EDTA tubes.
5a. Time centrifuge started for SST tube.
5b. Time centrifuge completed for SST tube.
5c. Time aliquots placed in −80° C. freezer for SST tube.
6. Date and time PAXgene tube placed in −80° C. freezer.

The instructions may comprise a procedure for shipping materials. The procedure may comprise one or more steps of:

For shipping:
1. Ship samples using shipping materials provided.
2. Store samples until they are packaged for shipment.
3. Ship Streck tubes on the day of collection at ambient temperature in ambient shipper.
4. Ship plasma, serum, buffy coat cryovial, and PAXgene at least monthly on dry ice in frozen shipper.
5. Assign a consecutive shipping number: 1, 2, 3 . . . .
6. Complete the following on a Sample Requisition Form:
6a. Date of shipment.
6b. Time samples packaged for shipment.
6c. Tracking number.
6d. All aliquots and applicable tubes included in shipment (Yes/No); if discrepancy (e.g., missing tube), list missing ID number of applicable discrepancy.
7. Place a copy of the Sample Requisition Form(s) between the inner cooler and the outer in an ambient shipping box prior to sealing.
8. Ship the samples overnight.

For frozen shipping of plasma, buffy coat and serum cryovials, and PAXgene tubes:
1. Prepare a frozen shipping box with at least 10-15 pounds of dry ice.
2. Place cryovial and PAXgene tubes in Ziploc bag containing absorbent sheet, ensuring that dry ice is packed around the sample tubes.
3. Place a copy of a Sample Requisition Form(s) between the inner cooler and the outer box prior to sealing.

For ambient shipping of Streck tubes:
1. Place Streck tubes in Ziploc bag containing absorbent sheet.
2. Place a copy of a Sample Requisition Form(s) between the inner cooler and the outer in an ambient shipping box prior to sealing.
3. Ship on the day of collection or, if collected late in the day, the next day.

### Example 3: Proteoform Detection in Deep Plasma Protemics Through Peptide Expression Correlation and Genomic Mapping

This example illustrates methods and systems for identification of proteins with distinct proteoforms. Protein isoforms from plasma samples of 80 healthy controls and 61 patients with early-stage non-small-cell lung cancer (NSCLC) were analyzed using a method of the present disclosure. More specifically, proteomes of 141 plasma samples were profiled using PROTEOGRAPH™ and LC-MS/MS. For all detected peptides within each protein group across all samples, a Pearson pairwise correlation of peptide abundances was calculated within each protein group. Different correlation methods can be used at this step such as, for example, Pearson correlation, Kendall rank correlation, Spearman correlation, the chatterjee correlation, the Point-Biserial correlation, and the like. To identify clusters of similarly abundant peptides, a silhouette method was applied to obtain an optimal number of clusters and K-means clustering on the correlation of peptide abundances was used. Different methods for determining an optimal number of clusters can be used in combination with clustering algorithms that requires the specification of number of clusters. Such methods of determining optimal number of clusters include, but are not limited to, Gap statistics, the Elbow Method, Calinski-Harabasz Index, Davies-Bouldin Index, the use of Dendrogram, Bayesian information criterion, and the like. The clustering method that benefits from specification of a number of clusters include, but is not limited to, any centroid-based clustering like K-means, K-medoid, k-modes, k-median, and the like. Clustering algorithms that require no specification of number of clusters can also be used to cluster peptides. Density-based Clustering like DBSCAN and DENCAST, Distribution-based Clustering like Gaussian Mixed Models and DBCLASD, and hierarchical clustering like DIANA and AGNES are all viable options to cluster peptides into groups for proteoform identification. A filtering step was then applied to ensure that the quantitative profile of peptides from different clusters are distinct. This filtering step can comprise calculating inter-cluster correlations between peptides within a cluster and peptides outside of a cluster. The average of all inter-cluster correlations can be lower than a certain threshold for the protein to be designated as a protein with distinct clusters. Otherwise, the derived clusters are said to belong to one cluster. The threshold is set at 0.4, for example, but can be set at different values. The threshold can be calculated based on the distribution of correlation of all proteins in the cohort, one standard deviation lower than the mean of the distribution can be used as the threshold. Next, in a separate process, peptides were mapped to protein isoforms from the ENSEMBL database. The presence of a proteoform was inferred if the known protein isoform explained the results of the peptide clustering.

Some identified proteoforms were found to display differential abundance profiles between the diseased and healthy subgroups, implying functional roles related to cancer. For example, one of the proteins with detected proteoforms, BMP1, can play both an activator and repressor role in cancer; the long and short proteoforms may contribute to the dual roles. In another example, endostatin proteoforms, a naturally occurring 20-kDa C-terminal short proteoform from type XVIII collagen (P39060), which can serve as an anti-angiogenic agent in treating NSCLC, were detected. These proteins would not have been detected as differentially abundant without the proteoform inference, because the quantitative signals from underlying peptides would have been merged from the distinct proteoforms in protein quantification. The ability to identify functionally relevant proteoforms offers increased opportunities to identify potential biomarkers for disease.

The use of multiple nanoparticles (NP), as uniquely enabled by PROTEOGRAPH™, can increase sensitivity of proteoform inference. For the same protein/protein group, it was observed different overlapping sets of peptides being detected from samples that are incubated with different NP. By analyzing the quantitative profile of these peptides, it suggests that different proteoforms are differentially captured by each distinct NP and by combining the results from multiple NPs the distinct proteoforms that are present in the sample can be elucidated.

PROTEOGRAPH™ can generate unbiased and deep plasma proteome profiles that enable the inference of biologically important proteoforms.

### Example 4: Proteoform Detection in Deep Plasma Proteomics Through Peptide Expression Correlation and Genomic Mapping

This example illustrates a method for identifying post-translational modification variants from proteomes.

Proteomes of plasma samples can be profiled using PROTEOGRAPH™ and LC-MS/MS to systematically infer proteoforms arising from alternative gene splicing and allelic variation. It is hypothesized that disease-associated proteoforms arising from alternative splicing would display differential abundance patterns.

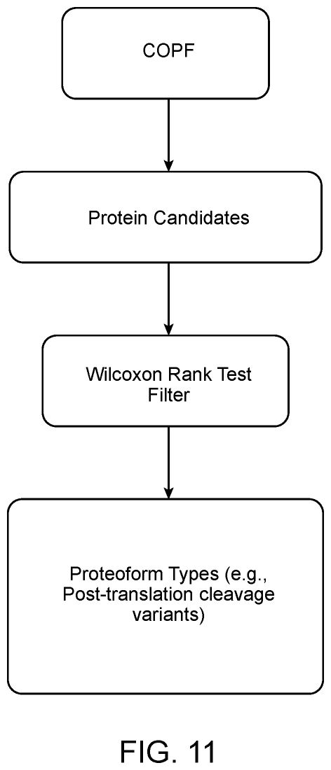

A proteome-wide differential abundance analysis can be performed. FIG. 11 illustrates a method for detecting proteoforms from proteomes, in accordance with some embodiments. To identify various proteoforms arising from alternative splicing, similarly abundant (i.e., covarying) peptides can be sequentially clustered by implemented COrrelation-based functional ProteoForm (COPF) assessment with minor adjustments and additional filtering steps. Pearson correlation can be calculated for pairwise peptides using logged intensity of the peptides across samples. K-means clustering can be then applied to the correlation matrix to group peptides into cluster/proteoforms. Proteoform score and p-value can be calculated according to COPF. K-means clustering and COPF's proteoform score may indicate when there exist significant clusters of proteins, inferring potential proteoforms.

Proteins with potential proteoforms according to COPF's proteoform score can be further filtered for certain type of proteoforms, for example, post-translational cleavage variants. A Post-Translational Cleavage Detection (PTCD) strategy is used, which employs the Wilcoxon's rank test to test for the statistical significance that peptides from one cluster/proteoform are disproportionally located on one terminus of the protein. Filtering for specifically post-translational cleaved proteoforms with Wilcoxon's rank test may show when there exist such proteoforms. The proteoforms may be associated with upregulated proteolytic peptides or downregulated proteolytic peptides.

Candidates can be mapped to proteoforms in a database, and the roles that candidates may play in a disease may be identified. To compare proteoform intensities, peptides intensities from proteoforms can be combined using MaxLFQ. Proteoforms can be associated with a clinically relevant dimension (e.g., cancer versus non-cancer). The intensities of the peptides can be combined for comparison at the protein level, however, the proteoforms may not be detectable at the protein level.

Using a modified COPF and Wilcoxon's rank test, post-translational cleaved proteoforms can be found. Differential expression of some signatures for a disease may be found at the proteoform level, but not at protein level.

### Example 5: Feature Selection for Early-Stage Disease Detection

This example illustrates a study design for identification of biomarkers for a disease.

Two groups of biological samples are studied. A first group of biological samples are obtained from individuals who are known to have developed the disease. A second group of biological samples are obtained from individuals who are known to be free of the disease.

Samples in both groups of biological samples are assayed using PROTEOGRAPH™ to determine biomolecule compositions for each sample.

A machine learning algorithm is designed such that it receives a biomolecule composition as input and output a predicted disease state. Various machine learning algorithms can be used. As one example, the machine learning algorithm is a random forest algorithm that (i) receives proteomic information that provides (a) peptide identifications, (b) intensities of the peptides of the peptide identifications, and (c) which particle of the PROTEOGRAPH™ the peptides were measured from. The machine learning algorithm can be trained with N-fold cross-validation. Once trained, the machine learning algorithm can be interrogated (e.g., by determining Shapley importance value) to determine which features of the proteomic information accounts for the variance in the proteomic information between the first group and the second group.

The accuracy of the machine learning algorithm at predicting the disease state is cross-validated. If the machine learning algorithm is able to detect correlations between the composition of the biosamples and the disease state, the accuracy will be better than random chance.

The machine learning algorithm may be analyzed to obtain features of the biomolecule composition that influence the output of the machine learning algorithm. These features are indicative of biomolecules that help differentiate between biosamples from subjects with the disease, and biosamples from subjects without the disease.

### Example 6: Protein Quantitative Trait Loci (pQTL) for Disease Detection

This example illustrates a method for identifying pQTLs.

A linear regression model with covariates was used to search pQTLs.

Phenotypes are represented by log 2 (Intensity) for each nanoparticle-protein group (NP-PG) combination.

A linear regression model is constructed with the form:

y=Gβ_{G}+Xβ_{X}+e

where y is NP-PG intensity, G is genotype, and X is covariate. β_{G} and β_{X} are learned coefficients from least-squares minimization. The model is evaluated to compute chi-squared goodness of fit statistics and p-value for 1 degree of freedom. Top PCs, sex, age, race, genotype array batch are used as covariates.

FDR is calculated by creating 20 shuffles of data, leading to 20 random intensities for each NP-PG in a sample. For a NP-PG, targets are SNP associations from the non-shuffled run and decoys are SNP associations from the 20 shuffles. The target/decoys are then ranked according to p-values to calculate FDR with ratio=20. The FDR has the form:

$$FDR = \frac{{N}_{decoy}}{{N}_{target} * ratio}$$

A pQTL is considered to be significant when the p-value is less than 1e⁻⁵ and the false discovery rate is less than 1e⁻². A pQTL is considered to be a cis-pQTL if the SNP is within +/−1 megabase pairs (Mbp) of a transcription start site (TSS) stopping at TSS of another gene, with a minimum of 5 kb up and 1 kb downstream. Otherwise, a pQTL is considered to be a trans-pQTL.

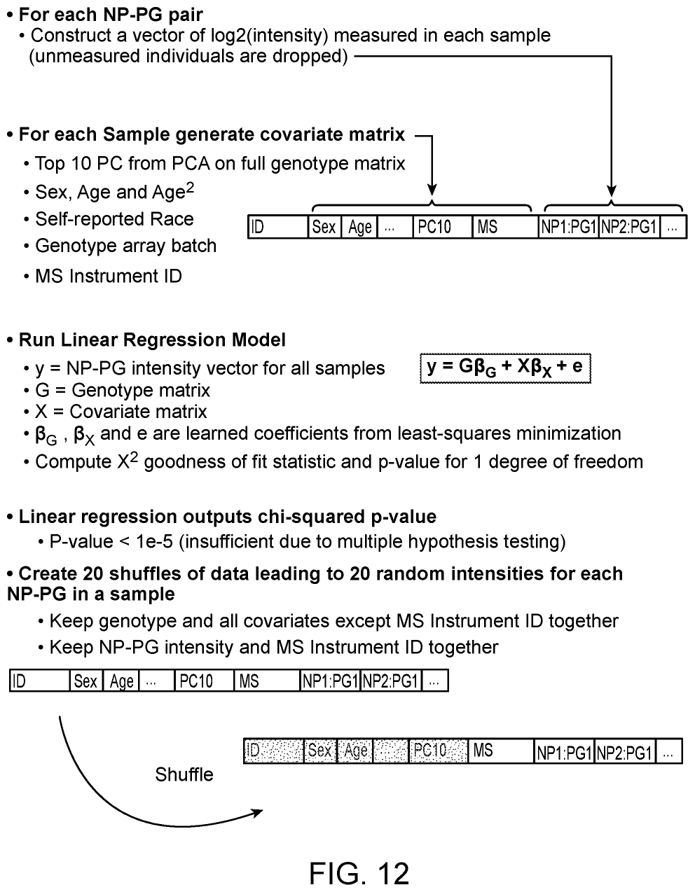

FIG. 12 schematically illustrates a calculation for pQTL association, in accordance with some embodiments.

### Example 7: Integrated Data Processing and Visualization Suite Leveraging Cloud Scalable Architecture for Large-Cohort Proteogenomics Data Analysis and Interpretation

This example illustrates a system and method for visualizing proteogenomic data. Comprehensive assessment of the flow of genetic information through multi-omic data integration can reveal the molecular consequences of genetic variation underlying human disease. Next generation sequencing (NGS) is used to identify genetic variants and characterize gene function (e.g., transcriptome and epigenome), while mass spectrometry is used to assess the proteome through characterization of protein abundances, modifications, and interactions. A scalable analysis platform (PROTEOGRAPH ANALYSIS SUITE™, PAS™) is used to perform proteogenomic data analyses through the integration of proteomics data derived from PROTEOGRAPH™ with genomic variant information derived from NGS experiments.

PAS™ was engineered to facilitate data handling continuum starting from data upload, search-engine processing, statistical filtering and protein quantification, to visualization tools for deriving functional and biological insight. PAS™ can automate data upload for direct transfer of datafiles from commercial LC-MS instrumentation, without user intervention. PAS™ features integration of popular open-source search engines, pre-installed analysis protocols, and setup wizards for seamless generation of results. To facilitate data quality and longitudinal performance monitoring across large cohort studies, PAS™ tracks 11 relevant assay and LC-MS metrics in an easy to interpret QC dashboard. Analyses results are compiled in a secure, browser-based portal using accessible, easy to understand formats including data tables and output graphics for reviewing and interpreting the underlying biology.

Proteomics and genomics data can be integrated using a variety of tools, many of which are operating system-dependent and available through command-line interfaces. Such limitation may act as a barrier for some researchers seeking to adapt new data analysis tools. PAS™ provides some of these tools in a user-friendly format that is usable on a variety of platforms with intuitive user interfaces.

PAS™ is compatible with variant call format (vcf) files from NGS workflows to enable personalized database searches. PAS™ supports both Data Independent Analysis (DIA) and Data Dependent Analysis (DDA) MS datafiles from all major proteomics vendor instrumentation. PAS™ can analyze VCF files generated from NGS pipelines in combination with mass spec data to identify peptide variants using customized search libraries. Using a cloud-based architecture, computational tasks are distributed for efficient and rapid analysis, significantly improving time to results. Visualizations including principal component analysis, hierarchical clustering, and heatmaps allow intuitive identification of underlying trends. To enable biological insights, differential expression analyses results are reported with interactive visualizations such as volcano plots, protein interaction maps, and protein-set enrichment. The integrated proteogenomics viewer allows variant IDs to be interpreted in the context of genomic coordinates, protein sequence, functional domains and features. Together, these results show the utility of PAS™ for seamless and fast proteomic and genomic data analysis.

### Example 8: NPs to Differentiate Proteoforms Based on Differential Peptide Signals

This example illustrates a method for inferring proteoforms (protein variants) from corona dynamics of nanoparticles.

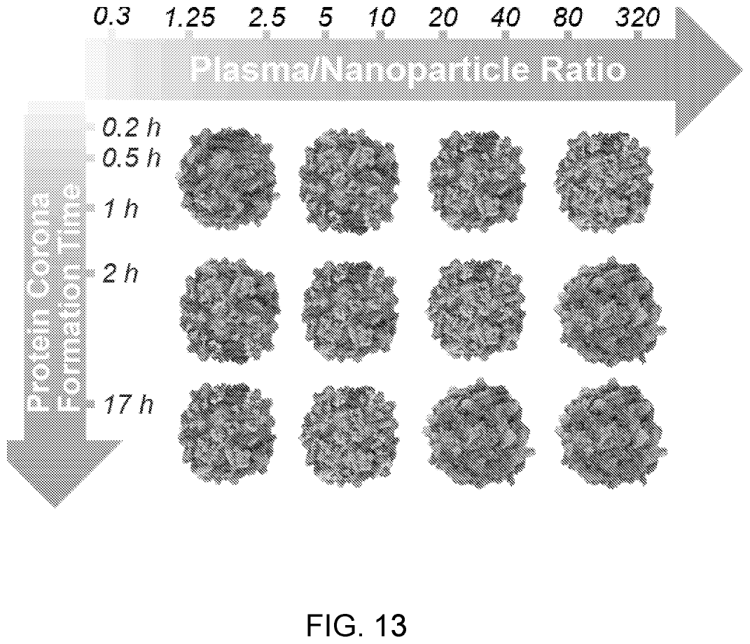

Proteoforms were inferred from corona dynamics of three nanoparticles. Protein to NP ratios, NP functionalization, and protein corona formation times were each varied with quantitative MS searching for peptides annotated for the same gene that behaved discordantly. Such an observation indicates presence of proteoforms that are differentially captured in NP-protein coronas. Results show that corona dynamics can resolve known and indicate novel protein variants. The protein corona conditions that were used are illustrated in FIG. 13: 3 NPs with different surface modifications, corona formation time course (10 min, 1 h, 2h, 17 h at 37 deg C.) for various Protein/NP ratios (1× to 100×) measuring the intensities of 3,184 individual protein groups (1% FDR at protein and peptide level) across 534 samples.

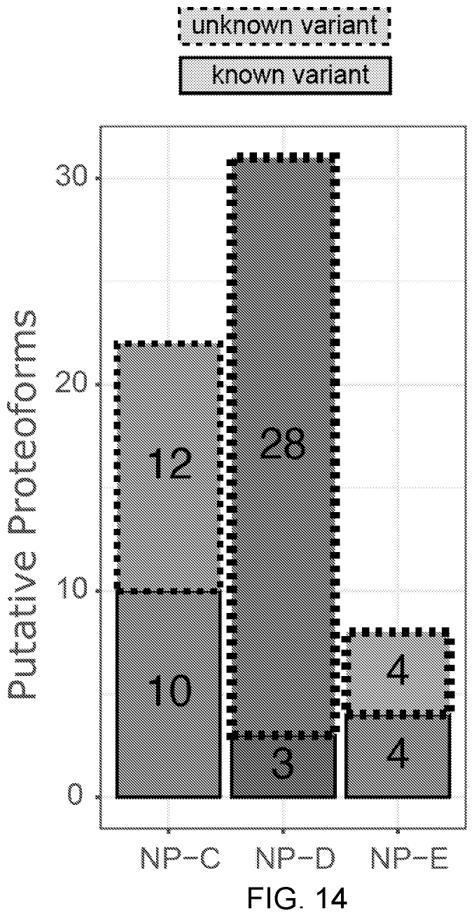

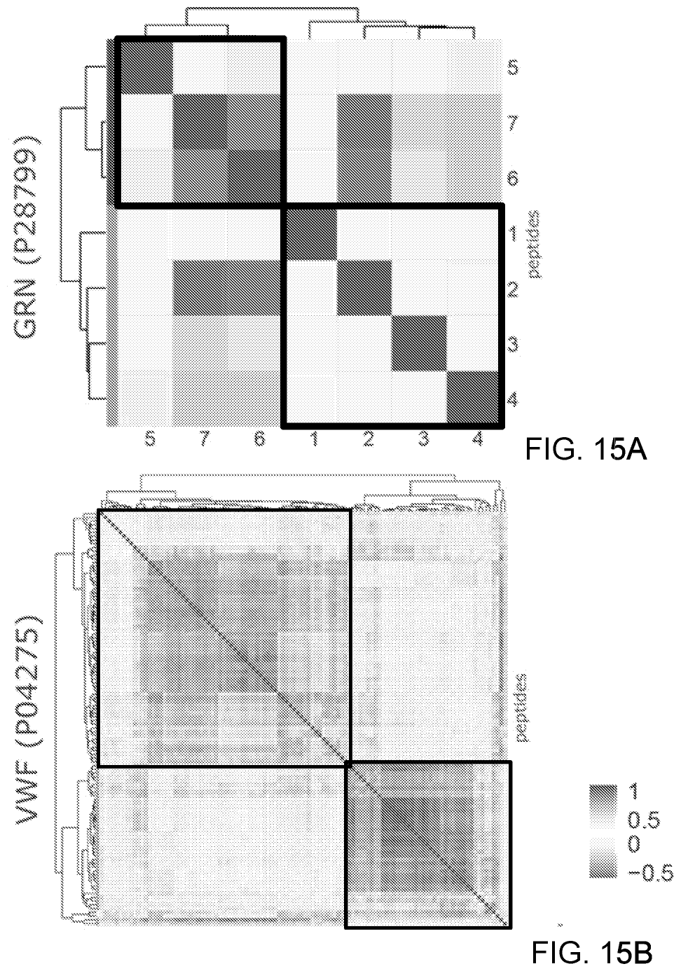

FIG. 14 shows the number of inferred proteoforms based on distinct peptide correlation (Pearson correlation) profiles across corona dynamics (time and P/NP ratio) for NP-C, NP-D, and NP-E with a false discovery rate<10% (Benjamini Hochberg, p.adjust( )). Solid line indicates the number of proteins for which differential peptide profiles map to known protein isoforms. FIG. 15A and FIG. 15B show examples of two peptide maps that indicate different protein isoforms, as they exhibit differential peptide correlation profiles which map to known protein isoforms.

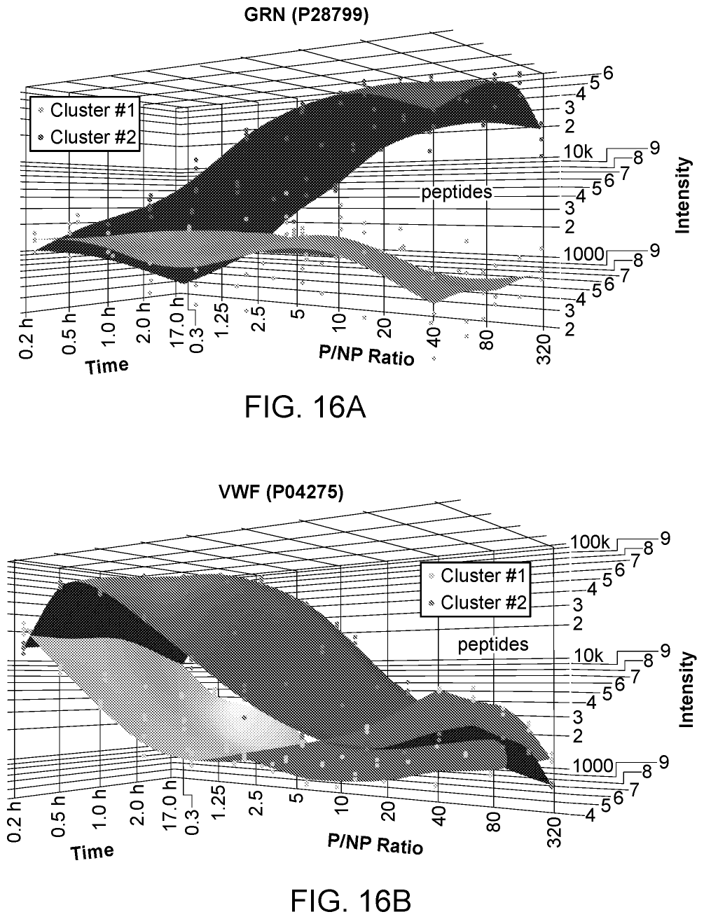

FIG. 16A and FIG. 16B show corona dynamic profiles that show the quantitative profiles of the protein isoform associated peptides. It shows that different protein isoforms exhibit different corona dynamics, indicating the capacity of NPs to capture distinct proteoforms. The results also demonstrate that, applying the disclosure in the present application, suitable protein-to-nanoparticle ratios, incubation conditions, and nanoparticle physicochemical properties may be used to select conditions that resolve different proteoforms in a biological sample.

TABLE 1
Sequences

| SEQ ID NO. | Sequence |
|---|---|
| SEQ ID NO: 1 | HPLKPDNQPFPQSVSESCPGK |
| SEQ ID NO: 2 | HPLKPDIQPFPQSVSESCPGK |
| SEQ ID NO: 3 | GDTSTIYTNCWVTGWGFSK |
| SEQ ID NO: 4 | GDTSTIYTNCWVTR |
| SEQ ID NO: 5 | ATGCTAGCGATCGTACGATCGATCGGTGCTTATTATTATAGCCG |
| | GTATATCGATGCCTA |
| SEQ ID NO: 6 | ATGCTAGCGATCGTACGATCGATCGGTGCTTATTATTATAGCCG |
| | GTATATCGATGCCTA |
| SEQ ID NO: 7 | MLAIVK |
| SEQ ID NO: 8 | RSIGAYYYS |
| SEQ ID NO: 9 | RYIDA |
| SEQ ID NO: 10 | EVYMPSSIFQDDFVIPDISEPGTWK |
| SEQ ID NO: 11 | PVAFSVVPTAAAAVSLK |
| SEQ ID NO: 12 | EYLIMGLDGATYDLEGHPQYLLDSNSWIEEMPSER |
| SEQ ID NO: 13 | AVGSGATFSHYYYMILSR |
| SEQ ID NO: 14 | ITPGKPYILTVPGHLDEMQLDIQAR |
| SEQ ID NO: 15 | LGQYASPTAK |
| SEQ ID NO: 16 | KYVLPNFEVK |
| SEQ ID NO: 17 | EFHLHLR |
| SEQ ID NO: 18 | SFFPENWLWR |
| SEQ ID NO: 19 | FQILTLWLPDSLTTWEIHGLSLSK |
| SEQ ID NO: 20 | YVSHFETEGPHVLLYFDSVPTSR |
| SEQ ID NO: 21 | LHLETDSLALVALGALDTALYAAGSK |
| SEQ ID NO: 22 | LNMGITDLQGLR |
| SEQ ID NO: 23 | VDVQAGACEGK |
| SEQ ID NO: 24 | GLCVATPVQLR |
| SEQ ID NO: 25 | NNVPCSPK |
| SEQ ID NO: 26 | FEQLELRPVLYNYLDK |
| SEQ ID NO: 27 | EGAIHREELVYELNPLDHR |
| SEQ ID NO: 28 | GHLFLQTDQPIYNPGQR |
| SEQ ID NO: 29 | FGLLDEDGK |
| SEQ ID NO: 30 | HLVPGAPFLLQALVR |
| SEQ ID NO: 31 | SHALQLNNR |
| SEQ ID NO: 32 | TYNVLDMK |
| SEQ ID NO: 33 | YVLPNFEVK |
| SEQ ID NO: 34 | GQIVFMNR |
| SEQ ID NO: 35 | LELSVDGAK |
| SEQ ID NO: 36 | VGLSGMAIADVTLLSGFHALR |
| SEQ ID NO: 37 | LLLFSPSVVHLGVPLSVGVQLQDVPR |
| SEQ ID NO: 38 | PYILTVPGHLDEMQLDIQAR |
| SEQ ID NO: 39 | GPEVQLVAHSPWLK |
| SEQ ID NO: 40 | GSFEFPVGDAVSK |
| SEQ ID NO: 41 | FGLLDEDGKK |
| SEQ ID NO: 42 | GLQDEDGYR |
| SEQ ID NO: 43 | TTNIQGINLLFSSR |
| SEQ ID NO: 44 | VDFTLSSERDFALLSLQVPLK |
| SEQ ID NO: 45 | LVNGQSHISLSK |
| SEQ ID NO: 46 | EELVYELNPLDHR |
| SEQ ID NO: 47 | VGDTLNLNLR |
| SEQ ID NO: 48 | EMSGSPASGIPVK |
| SEQ ID NO: 49 | PVQGVAYVR |
| SEQ ID NO: 50 | DFALLSLQVPLK |
| SEQ ID NO: 51 | VEYGFQVK |
| SEQ ID NO: 52 | VFALDQK |
| SEQ ID NO: 53 | AEFQDALEK |
| SEQ ID NO: 54 | VDFTLSSER |
| SEQ ID NO: 55 | ITQVLHFTK |
| SEQ ID NO: 56 | GLEEELQFSLGSK |
| SEQ ID NO: 57 | VVEEQESR |
| SEQ ID NO: 58 | ALEILQEEDLIDEDDIPVR |
| SEQ ID NO: 59 | ASAGLLGAHAAAITAYALTLTK |
| SEQ ID NO: 60 | ADGSYAAWLSR |
| SEQ ID NO: 61 | QGSFQGGFR |
| SEQ ID NO: 62 | AEMADQASAWLTR |
| SEQ ID NO: 63 | NPSDPMPQAPALWIETTAYALLHLLLHEGK |
| SEQ ID NO: 64 | SMQGGLVGNDETVALTAFVTIALHHGLAVFQDEGAEPLK |
| SEQ ID NO: 65 | TEQWSTLPPETK |
| SEQ ID NO: 66 | VLSLAQEQVGGSPEK |
| SEQ ID NO: 67 | TLEIPGNSDPNMIPDGDFNSYVR |
| SEQ ID NO: 68 | DHAVDLIQK |
| SEQ ID NO: 69 | ANSFLGEK |
| SEQ ID NO: 70 | DSSTWLTAFVLK |
| SEQ ID NO: 71 | LQETSNWLLSQQQADGSFQDPCPVLDR |
| SEQ ID NO: 72 | STQDTVIALDALSAYWIASHTTEER |
| SEQ ID NO: 73 | VTASDPLDTLGSEGALSPGGVASLLR |
| SEQ ID NO: 74 | EFMSQGNK |
| SEQ ID NO: 75 | QRPPDLDTSSNAVDLLFFTDESGDSR |
| SEQ ID NO: 76 | YTTTMGVNTYK |
| SEQ ID NO: 77 | TLDEFTIIQNLQPQYQFR |
| SEQ ID NO: 78 | LFGEVTSPLFPK |
| SEQ ID NO: 79 | YTTEIIK |
| SEQ ID NO: 80 | PYPNNFETTTVITVPTGYR |
| SEQ ID NO: 81 | NLPNGDFR |
| SEQ ID NO: 82 | VLNYVDWIK |
| SEQ ID NO: 83 | PVNPVEQR |
| SEQ ID NO: 84 | GYGFYTK |
| SEQ ID NO: 85 | DYFIATCK |
| SEQ ID NO: 86 | GLTLHLK |
| SEQ ID NO: 87 | LPVANPQACENWLR |
| SEQ ID NO: 88 | ESEQGVYTCTAQGIWK |
| SEQ ID NO: 89 | LVFQQFDLEPSEGCFYDYVK |
| SEQ ID NO: 90 | FLEPFDIDDHQQVHCPYDQLQIYANGK |
| SEQ ID NO: 91 | LIAPVAEEEATVPNNK |
| SEQ ID NO: 92 | SADTLWDIQK |
| SEQ ID NO: 93 | FIIPQIVK |
| SEQ ID NO: 94 | SLADELALVDVLEDK |
| SEQ ID NO: 95 | DYSVTANSK |
| SEQ ID NO: 96 | LKDDEVAQLK |
| SEQ ID NO: 97 | GLTSVINQK |
| SEQ ID NO: 98 | IVVVTAGVR |
| SEQ ID NO: 99 | MVVESAYEVIK |
| SEQ ID NO: 100 | VIGSGCNLDSAR |
| SEQ ID NO: 101 | GMYGIENEVFLSLPCILNAR |
| SEQ ID NO: 102 | IHPVSTMVK |
| SEQ ID NO: 103 | SEDAGGAEGETPHMLLRPHVFMPEVTPDMDYLPR |
| SEQ ID NO: 104 | IFSEDDDYIDIVDSLSVSPTDSDVSAGNILQLFHGK |

### Numbered Embodiments

The following list of numbered embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for assaying a biological sample, comprising: assaying a set of nucleic acids from the biological sample to obtain genotypic information of the biological sample; generating, based at least in part on the genotypic information, a set of expressible proteoforms that can be expressed from the set of nucleic acids; assaying a set of polyamino acids from the biological sample to generate proteomic information of the biological sample, wherein the proteomic information comprises a set of identifications for the set of polyamino acids; and mapping the set of identifications to the set of expressible proteoforms, thereby determining a set of expressed proteoforms in the biological sample. Embodiment 2. The method of embodiment 1, wherein the set of nucleic acids comprises an exome of the biological sample and the genotypic information comprises an exome sequence of the biological sample. Embodiment 3. The method of embodiment 1 or 2, wherein the set of proteoforms comprise peptide variants, protein variants, or both. Embodiment 4. The method of any one of embodiments 1-3, wherein the set of expressible proteoforms comprise splicing variants, allelic variants, post-translation modification variants, or any combination thereof. Embodiment 5. The method of any one of embodiments 1-4, wherein the post-translation modification variants comprise post-translational cleavage variants, phosphorylated variants, or any combination thereof. Embodiment 6. The method of any one of embodiments 1-5, wherein the set of polyamino acids comprise a set of peptide fragments derived from a set of proteins expressed in the biological sample. Embodiment 7. The method of embodiment 6, wherein the set of peptide fragments are derived by trypsinization. Embodiment 8. The method of embodiment 7, wherein the set of peptide fragments comprise tryptic peptide fragments, semi-tryptic peptide fragments, or both. Embodiment 9. The method of any one of embodiments 1-8, further comprising filtering the set of expressible proteoforms for a proteoform type. Embodiment 10. The method of embodiment 9, wherein the filtering is based on a statistical significance value that an expressible proteoform in the set of expressible proteoforms comprises the proteoform type. Embodiment 11. The method of embodiment 10, wherein the proteoform type is a splicing variant. Embodiment 12. The method of embodiment 11, wherein the statistical significance value is based on a probability that a sequence of the expressible proteoform comprises a reordered amino acid sequence of another expressible proteoform from the same protein group as the expressible proteoform. Embodiment 13. The method of embodiment 11, wherein the statistical significance value is based on a probability that a sequence of the expressible proteoform comprises a subsequence of an amino acid sequence another expressible proteoform from the same protein group as the expressible proteoform. Embodiment 14. The method of embodiment 10, wherein the proteoform type is an allelic variant. Embodiment 15. The method of embodiment 14, wherein the statistical significance value is based on a probability that a sequence of the expressible proteoform comprises an amino acid substitution in an amino acid sequence of another expressible proteoform from the same protein group. Embodiment 16. The method of embodiment 10, wherein the proteoform type is a post-translational cleavage variant. Embodiment 17. The method of embodiment 16, wherein the statistical significance value is based on a probability that peptide fragments of the expressible proteoform is localized on one terminus of another expressible proteoform from the same protein group. Embodiment 18. The method of embodiment 10, wherein the proteoform type is a phosphorylated variant. Embodiment 19. The method of embodiment 18, wherein the statistical significance value is based on a probability that a sequence of the expressible proteoform comprises a phosphorylated amino acid. Embodiment 20. The method of any one of embodiments 1-19, wherein the set of polyamino acids comprise a set of proteins expressed in the biological sample. Embodiment 21. The method of any one of embodiments 1-20, wherein the proteomic information comprises a set of peptide intensities detected from assaying the set of polyamino acids. Embodiment 22. The method of any one of embodiments 1-21, wherein the set of identifications comprises protein group identifications for the set of polyamino acids. Embodiment 23. The method of any one of embodiments 1-22, wherein the set of identifications comprises amino acid sequences for the set of polyamino acids. Embodiment 24. The method of any one of embodiments 1-23, wherein the set of identifications comprises post-translational modifications for the set of polyamino acids. Embodiment 25. The method of any one of embodiments 1-24, wherein the mapping comprises matching an identification in the set of identifications to an expressible proteoform in the set of expressible proteoforms, thereby determining that the matched expressible proteoform is an expressed proteoform in the biological sample. Embodiment 26. The method of any one of embodiments 1-25, further comprising associating the set of expressed proteoforms with a biological state of the biological sample. Embodiment 27. The method of embodiment 26, wherein the associating is based at least partially on the expression levels of each proteoform in the set of expressed proteoforms. Embodiment 28. The method of embodiment 26 or 27, further comprising associating the genotypic information with the biological state of the biological sample. Embodiment 29. The method of any one of embodiments 1-28, wherein the set of polyamino acids are derived from the biological sample using at least one untargeted assay. Embodiment 30. The method of any one of embodiments 1-29, wherein the set of polyamino acids comprise a dynamic range of at least 5, 6, 7, 8, 9, 10, 11, or 12 orders of magnitude in the biological sample. Embodiment 31. The method of embodiment 29 or 30, wherein the at least one untargeted assay comprises: providing a plurality of surface regions comprising a plurality of surface types; contacting the biological sample with the plurality of surface regions to yield a set of adsorbed biomolecules on the plurality of surface regions; and desorbing, from the plurality of surface regions, at least a portion of the set of adsorbed biomolecules to yield the set of polyamino acids. Embodiment 32. The method of embodiment 31, wherein the plurality of surface regions are disposed on a single continuous surface. Embodiment 33. The method of embodiment 31, wherein the plurality of surface regions are disposed on a plurality of discrete surfaces. Embodiment 34. The method of embodiment 33, wherein the plurality of discrete surfaces are surfaces of a plurality of particles. Embodiment 35. The method of any one of embodiments 29-34, wherein the at least one untargeted assay has a false discovery rate of at most about 5%, 4%, 3%, 2%, 1%, 0.9%, 0.8%, 0.7%, 0.6%, 0.5%, 0.4%, 0.3%, 0.2%, or 0.1%. Embodiment 36. The method of any one of embodiments 29-35, wherein the set of expressed proteoforms comprises at least about 10, 20, 30, 40, 50, 60, 70, 80, 90, 100, 200, 300, 400, 500, 600, 700, 800, 900, or 1,000 expressed proteoforms.

Embodiment 37. A method for assaying a biological sample, comprising: assaying a set of polyamino acids from the biological sample to generate proteomic information of the biological sample, wherein the proteomic information comprises a set of polyamino acid identifications for the set of polyamino acids; assaying a set of nucleic acids from the biological sample to obtain genotypic information of the biological sample, wherein the genotypic information comprises one or more nucleic acid sequences; and determining expression levels of one or more regions in the one or more nucleic acid sequences, based at least partially on the set of polyamino acid identifications. Embodiment 38. The method of embodiment 37, wherein the set of polyamino acids comprises a set of peptide fragments derived from a set of proteins expressed in the biological sample. Embodiment 39. The method of embodiment 37 or 38, wherein the set of polyamino acid identifications comprises protein group identifications or amino acid sequences for the set of polyamino acids. Embodiment 40. The method of any one of embodiments 37-39, wherein the set of nucleic acids is an exome of the biological sample and the genotypic information comprises an exome sequence of the biological sample. Embodiment 41. The method of embodiment 40, wherein the one or more regions are one or more exons in the exome sequence. Embodiment 42. The method of any one of embodiments 37-41, wherein the set of polyamino acids are derived from the biological sample using at least one untargeted assay. Embodiment 43. The method of any one of embodiments 37-42, wherein the set of polyamino acids comprise a dynamic range of at least 5, 6, 7, 8, 9, 10, 11, or 12 orders of magnitude in the biological sample. Embodiment 44. The method of any one of embodiments 37-43, wherein the at least one untargeted assay comprises: providing a plurality of surface regions comprising a plurality of surface types; contacting the biological sample with the plurality of surface regions to yield a set of adsorbed biomolecules on the plurality of surface regions; and desorbing, from the plurality of surface regions, at least a portion of the set of adsorbed biomolecules to yield the set of polyamino acids. Embodiment 45. The method of embodiment 44, wherein the plurality of surface regions are disposed on a single continuous surface. Embodiment 46. The method of embodiment 44, wherein the plurality of surface regions are disposed on a plurality of discrete surfaces. Embodiment 47. The method of embodiment 46, wherein the plurality of discrete surfaces are surfaces of a plurality of particles. Embodiment 48. The method of any one of embodiments 37-47, wherein the determining comprises identifying one or more base positions in the one or more nucleic acid sequences that covaries with at least one element in the proteomic information. Embodiment 49. The method of embodiment 48, wherein the one or more base positions comprise a single nucleotide polymorphism. Embodiment 50. The method of embodiment 48 or 49, wherein the at least one element comprises a polyamino acid identification in the set of polyamino acid identifications and a polyamino acid intensity measured using the untargeted assay. Embodiment 51. The method of any one of embodiments 48-50, wherein the determining further comprises filtering the one or more base positions when a statistical significance value for the one or more base pair positions is less than a threshold statistical significance value. Embodiment 52. The method of embodiment 51, wherein the statistical significance value is a p-value. Embodiment 53. The method of embodiment 51 or 52, wherein the threshold statistical significance value is 1e⁻⁵. Embodiment 54. The method of any one of embodiments 48-53, wherein the determining further comprises filtering the one or more base positions when a false discovery rate for the one or more base pair positions is less than a threshold false discovery rate. Embodiment 55. The method of embodiment 54, wherein the false discovery rate is determined by: shuffling the proteomic data to generate a shuffled proteomic data; identifying one or more decoy base positions in a shuffled proteomic data that covaries with at least one element in the proteomic information; and normalizing the number of the one or more decoy base positions by the number of the one or more base positions. Embodiment 56. The method of any one of embodiments 48-55, further comprising classifying the one or more base positions as a cis-pQTL or a trans-pQTL based on a distance between the one or more base positions and a gene that encodes a polyamino acid comprising the polyamino acid identification. Embodiment 57. The method of embodiment 56, wherein the one or more base positions are classified as a cis-pQTL when the distance is less than 1 megabase pairs (Mbp) of a transcription start site of the gene. Embodiment 58. The method of embodiment 56 or 57, wherein the one or more regions in the one or more nucleic acid sequences comprises the gene that encodes a polyamino acid comprising the polyamino acid identification.

Embodiment 59. A method for identifying a differentially expressed polyamino acid, comprising: obtaining a plurality of polyamino acids from a plurality of biological samples, wherein the plurality of biological samples are differential in at least one clinically relevant dimension; assaying the plurality of polyamino acids, using at least one untargeted assay, to generate a plurality of identifications for the plurality of polyamino acids; and identifying at least one polyamino acid in the plurality of polyamino acids that is differentially expressed or abundant in the at least one clinically relevant dimension. Embodiment 60. The method of embodiment 59, wherein the at least one clinically relevant dimension is a disease state. Embodiment 61. The method of embodiment 60, wherein the disease state is a presence of cancer or an absence of cancer. Embodiment 62. The method of embodiment 60, wherein the disease state is a stage of cancer. Embodiment 63. The method of any one of embodiments 59-62, wherein the plurality of polyamino acids are peptide fragments derived from proteins expressed in the plurality of biological samples. Embodiment 64. The method of any one of embodiments 59-63, wherein the set of polyamino acids comprise a dynamic range of at least 5, 6, 7, 8, 9, 10, 11, or 12 orders of magnitude in the biological sample. Embodiment 65. The method of any one of embodiments 59-64, wherein the at least one untargeted assay comprises: providing a plurality of surface regions comprising a plurality of surface types; contacting the biological sample with the plurality of surface regions to yield a set of adsorbed biomolecules on the plurality of surface regions; and desorbing, from the plurality of surface regions, at least a portion of the set of adsorbed biomolecules to yield the set of polyamino acids. Embodiment 66. The method of embodiment 65, wherein the plurality of surface regions are disposed on a single continuous surface. Embodiment 67. The method of embodiment 65, wherein the plurality of surface regions are disposed on a plurality of discrete surfaces. Embodiment 68. The method of embodiment 67, wherein the plurality of discrete surfaces are surfaces of a plurality of particles.

Embodiment 69. A method for assaying a biological sample, comprising: (a) assaying a set of peptides from the biological sample using spectral data to generate proteomic information of the biological sample, wherein the proteomic information comprises a set of identifications for the set of peptides; (b) identifying a set of protein groups based at least in part on the spectral data of the set of peptides; (c) identifying one or more sets of peptides that are correlated in abundance for a given protein group in the set of protein groups; and (d) mapping the set of peptides to a database of human genes with isoform information, thereby determining a set of proteoforms that result in the set of peptides. Embodiment 70. The method of embodiment 69, wherein the spectral data comprises mass spectrometry data. Embodiment 71. The method of embodiment 69 or 70, wherein the identifying the one or more sets of peptides comprises computing a correlation of abundances of each peptide across the biological sample. Embodiment 72. The method of any one of embodiments 69-71, wherein the identifying the one or more sets of peptides comprises computing a correlation of abundances of each peptide across a plurality of biological samples or clustering based on peptides' correlations. Embodiment 73. The method of any one of embodiments 69-72, further comprising, subsequent to (c), identifying a first set of peptides that are correlated in abundance; further comprising identifying a second set of peptides that are correlated in abundance; and further comprising applying a filtering step to confirm that the set of peptides are distinct from each other. Embodiment 74. The method of embodiment 73, further comprising identifying more than two sets of peptides that are correlated in abundance, and applying a filtering step to confirm that the more than two sets of peptides are distinct from each other. Embodiment 75. The method of embodiment 73 or 74, wherein the first set of peptides comprise a first proteoform, and the second set of peptides comprise a second proteoform, wherein the first proteoform and the second proteoform are expressed from a same locus of exons. Embodiment 76. The method of any one of embodiments 69-75, further comprising filtering the set of proteoforms for a proteoform type. Embodiment 77. The method of embodiment 76, wherein the filtering is based on a statistical significance value that a proteoform in the set of proteoforms comprises the proteoform type. Embodiment 78. The method of embodiment 77, wherein the proteoform type is a splicing variant. Embodiment 79. The method of embodiment 77 or 78, wherein the statistical significance value is based on a probability that a sequence of the proteoform comprises a reordered amino acid sequence of another proteoform from the same protein group. Embodiment 80. The method of any one of embodiments 77-79, wherein the statistical significance value is based on a probability that a sequence of the proteoform comprises a subsequence of an amino acid sequence another proteoform from the same protein group. Embodiment 81. The method of embodiment 77, wherein the proteoform type is an allelic variant. Embodiment 82. The method of embodiment 81, wherein the statistical significance value is based on a probability that a sequence of the proteoform comprises an amino acid substitution in an amino acid sequence of another proteoform from the same protein group. Embodiment 83. The method of embodiment 77, wherein the proteoform type is a post-translational cleavage variant. Embodiment 84. The method of embodiment 83, wherein the statistical significance value is based on a probability that peptide fragments of the proteoform is localized on one terminus of another proteoform from the same protein group. Embodiment 85. The method of embodiment 77, wherein the proteoform type is a phosphorylated variant. Embodiment 86. The method of embodiment 85, wherein the statistical significance value is based on a probability that a sequence of the proteoform comprises a phosphorylated amino acid. Embodiment 87. The method of any one of embodiments 69-86, wherein the biological sample comprises a plasma sample derived from a subject afflicted with a non-small cell lung cancer. Embodiment 88. The method of any one of embodiments 69-87, wherein an identified proteoform is associated with a disease. Embodiment 89. The method of any one of embodiments 69-88, wherein the set of proteoforms comprise peptide variants, protein variants, or both. Embodiment 90. The method of any one of embodiments 69-89, wherein the set of proteoforms comprise splicing variants, allelic variants, post-translation modification variants, or any combination thereof. Embodiment 91. The method of any one of embodiments 69-90, wherein the database of human genes comprises an ENSEMBL database with isoform information.

Embodiment 92. A computer-implemented method, implementing any one of the methods of embodiments 1-91 in a computer. Embodiment 93. A computer program product comprising a computer-readable medium having computer-executable code encoded therein, the computer-executable code adapted to be executed to implement any one of the methods of embodiments 1-91. Embodiment 94. A non-transitory computer-readable storage media encoded with a computer program including instructions executable by one or more processors to implement any one of the methods of embodiments 1-91. Embodiment 95. A computer-implemented system comprising: a digital processing device comprising: at least one processor, an operating system configured to perform executable instructions, a memory, and a computer program including instructions executable by the digital processing device to perform any one of the methods of embodiments 1-91.

Embodiment 96. A computer-implemented method for assaying a biological sample, comprising: retrieving genotypic information associated with the biological sample from a database; generating, based at least in part on the genotypic information, a set of expressible proteoforms that can be expressed from the set of nucleic acids; retrieving assay data for a set of polyamino acids from the biological sample from a database; generating proteomic information of the biological sample using the assay data, wherein the proteomic information comprises a set of identifications for the set of polyamino acids; and mapping the set of identifications to the set of expressible proteoforms, thereby determining a set of expressed proteoforms in the biological sample. Embodiment 97. The method of embodiment 96, wherein the genotypic information comprises whole genome sequence data associated with the biological sample. Embodiment 98. The method of embodiment 96 or 97, wherein the genotypic information comprises exome sequence data, transcriptome sequence data, epigenome sequence data, or any combination thereof associated with the biological sample. Embodiment 99. The method of any one of embodiments 96-98, wherein the proteomic information further comprises abundance data for the set of polyamino acids. Embodiment 100. The method of any one of embodiments 96-99, wherein the assay data comprises mass spectrometry data. Embodiment 101. The method of any one of embodiments 96-99, wherein the assay data comprises protein sequencing data. Embodiment 101. The method of any one of embodiments 96-101, wherein the assay data comprises a quantity of peptides obtained by incubating the biological sample with a surface to form a protein corona and digesting proteins from the protein corona.

Embodiment 102. A computer-implemented method for assaying a biological sample, comprising: retrieving genotypic information associated with the biological sample from a database, wherein the genotypic information comprises one or more nucleic acid sequences; retrieving assay data for a set of polyamino acids from the biological sample from a database; generating proteomic information of the biological sample using the assay data, wherein the proteomic information comprises a set of identifications for the set of polyamino acids; determining expression levels of one or more regions in the one or more nucleic acid sequences, based at least partially on the set of identifications. Embodiment 103. The method of embodiment 102, wherein the genotypic information comprises whole genome sequence data associated with the biological sample. Embodiment 104. The method of embodiment 102 or 103, wherein the genotypic information comprises exome sequence data, transcriptome sequence data, epigenome sequence data, or any combination thereof associated with the biological sample. Embodiment 105. The method of any one of embodiments 102-104, wherein the assay data comprises mass spectrometry data. Embodiment 106. The method of any one of embodiments 102-104, wherein the assay data comprises protein sequencing data. Embodiment 107. The method of any one of embodiments 102-106, wherein the assay data comprises a quantity of peptides obtained by incubating the biological sample with a surface to form a protein corona and digesting proteins from the protein corona.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method for assaying a biological sample, comprising:
(a) assaying a set of nucleic acids from the biological sample to obtain genotypic information of the biological sample;
(b) generating, based at least in part on the genotypic information, a set of expressible proteoforms that can be expressed from the set of nucleic acids;
(c) assaying a set of polyamino acids from the biological sample to generate proteomic information of the biological sample, wherein the proteomic information comprises a set of identifications for the set of polyamino acids; and
(d) mapping the set of identifications to the set of expressible proteoforms, thereby determining a set of expressed proteoforms in the biological sample.

2. The method of claim 1, wherein the assaying comprises mass spectrometry or protein sequencing.

3. The method of claim 1, wherein the set of polyamino acids comprise a set of peptide fragments derived from a set of proteins expressed in the biological sample.

4. The method of claim 1, further comprising filtering the set of expressible proteoforms for a proteoform type.

5. The method of claim 4, wherein the filtering is based on a statistical significance value that an expressible proteoform in the set of expressible proteoforms comprises the proteoform type.

6. The method of claim 5, wherein the proteoform type is a splicing variant.

7. The method of claim 6, wherein the statistical significance value is based on a probability that a sequence of the expressible proteoform comprises a reordered amino acid sequence of another expressible proteoform from the same protein group as the expressible proteoform.

8. The method of claim 6, wherein the statistical significance value is based on a probability that a sequence of the expressible proteoform comprises a subsequence of an amino acid sequence another expressible proteoform from the same protein group as the expressible proteoform.

9. The method of claim 5, wherein the proteoform type is an allelic variant.

10. The method of claim 9, wherein the statistical significance value is based on a probability that a sequence of the expressible proteoform comprises an amino acid substitution in an amino acid sequence of another expressible proteoform from the same protein group.

11. The method of claim 5, wherein the proteoform type is a post-translational cleavage variant.

12. The method of claim 11, wherein the statistical significance value is based on a probability that peptide fragments of the expressible proteoform is localized on one terminus of another expressible proteoform from the same protein group.

13. The method of claim 5, wherein the proteoform type is a phosphorylated variant.

14. The method of claim 13, wherein the statistical significance value is based on a probability that a sequence of the expressible proteoform comprises a phosphorylated amino acid.

15. The method of claim 1, wherein the mapping comprises matching an identification in the set of identifications to an expressible proteoform in the set of expressible proteoforms, thereby determining that the matched expressible proteoform is an expressed proteoform in the biological sample.

16. The method of claim 1, wherein the set of polyamino acids comprise a dynamic range of at least 5, 6, 7, 8, 9, 10, 11, or 12 orders of magnitude in the biological sample.

17. The method of claim 16, wherein the assaying comprises:
(a) providing a plurality of surface regions comprising a plurality of surface types;
(b) contacting the biological sample with the plurality of surface regions to yield a set of adsorbed biomolecules on the plurality of surface regions; and
(c) desorbing, from the plurality of surface regions, at least a portion of the set of adsorbed biomolecules to yield the set of polyamino acids.

18. The method of claim 17, wherein the plurality of surface regions are disposed on a single continuous surface.

19. The method of claim 17, wherein the plurality of surface regions are disposed on a plurality of discrete surfaces, and wherein the plurality of discrete surfaces are surfaces of a plurality of particles.

20. The method of claim 1, wherein the set of expressed proteoforms comprises at least about 10, 20, 30, 40, 50, 60, 70, 80, 90, 100, 200, 300, 400, 500, 600, 700, 800, 900, or 1,000 expressed proteoforms.

21. A method for assaying a biological sample, comprising:
(a) assaying a set of polyamino acids from the biological sample to generate proteomic information of the biological sample, wherein the proteomic information comprises a set of polyamino acid identifications for the set of polyamino acids;
(b) assaying a set of nucleic acids from the biological sample to obtain genotypic information of the biological sample, wherein the genotypic information comprises one or more nucleic acid sequences; and
(c) determining expression levels of one or more regions in the one or more nucleic acid sequences, based at least partially on the set of polyamino acid identifications.

22. A method for identifying a differentially expressed polyamino acid, comprising:
(a) obtaining a plurality of polyamino acids from a plurality of biological samples, wherein the plurality of biological samples are differential in at least one clinically relevant dimension;
(b) assaying the plurality of polyamino acids, using at least one untargeted assay, to generate a plurality of identifications for the plurality of polyamino acids; and
(c) identifying at least one polyamino acid in the plurality of polyamino acids that is differentially expressed or abundant in the at least one clinically relevant dimension.

23. A method for assaying a biological sample, comprising:
(a) assaying a set of peptides from the biological sample using spectral data to generate proteomic information of the biological sample, wherein the proteomic information comprises a set of identifications for the set of peptides;
(b) identifying a set of protein groups based at least in part on the spectral data of the set of peptides;
(c) identifying one or more sets of peptides that are correlated in abundance for a given protein group in the set of protein groups; and
(d) mapping the set of peptides to a database of human genes with isoform information, thereby determining a set of proteoforms that result in the set of peptides.

24. A computer-implemented method for assaying a biological sample, comprising:
(a) retrieving genotypic information associated with the biological sample from a database;
(b) generating, based at least in part on the genotypic information, a set of expressible proteoforms that can be expressed from the set of nucleic acids;
(c) retrieving assay data for a set of polyamino acids from the biological sample from a database;
(d) generating proteomic information of the biological sample using the assay data, wherein the proteomic information comprises a set of identifications for the set of polyamino acids; and
(e) mapping the set of identifications to the set of expressible proteoforms, thereby determining a set of expressed proteoforms in the biological sample.

25. The method of claim 24, wherein the genotypic information comprises whole genome sequence data associated with the biological sample.

26. The method of claim 24, wherein the genotypic information comprises exome sequence data, transcriptome sequence data, epigenome sequence data, or any combination thereof associated with the biological sample.

27. The method of claim 24, where the proteomic information further comprises abundance data for the set of polyamino acids.

28. A computer-implemented method for assaying a biological sample, comprising:
(a) retrieving genotypic information associated with the biological sample from a database, wherein the genotypic information comprises one or more nucleic acid sequences;
(b) retrieving assay data for a set of polyamino acids from the biological sample from a database;
(c) generating proteomic information of the biological sample using the assay data, wherein the proteomic information comprises a set of identifications for the set of polyamino acids;
(d) determining expression levels of one or more regions in the one or more nucleic acid sequences, based at least partially on the set of identifications.

29. The method of claim 28, wherein the genotypic information comprises whole genome sequence data associated with the biological sample.

30. The method of claim 28, wherein the genotypic information comprises exome sequence data, transcriptome sequence data, epigenome sequence data, or any combination thereof associated with the biological sample.
